(19) United States
(12) Patent Application Publication
(10) Pub. No.: US 2024/0216474 A1
(43) Pub. Date: Jul. 4, 2024

(54) ACTIVATABLE INTERFERON POLYPEPTIDES AND METHODS OF USE THEREOF

(71) Applicant: Werewolf Therapeutics, Inc., Watertown, MA (US)

(72) Inventors: William WINSTON, West Newton, MA (US); Daniel HICKLIN, Boston, MA (US); Jose Andres SALMERON-GARCIA, Acton, MA (US); Cynthia SEIDEL-DUGAN, Belmont, MA (US); Heather BRODKIN, West Newton, MA (US); Philipp STEINER, Boston, MA (US)

(21) Appl. No.: 18/440,841

(22) Filed: Feb. 13, 2024

Related U.S. Application Data
(63) Continuation of application No. PCT/US2022/040564, filed on Aug. 17, 2022.
Application No. PCT/US2022/040564 is a continuation of application No. 63/234,284, filed on Aug. 18, 2021.

(51) Int. Cl. A61K 38/21; A61K 47/64; A61K 47/65; A61K 47/68; A61P 35/00

(52) U.S. Cl. CPC A61K 38/217; A61K 38/212; A61K 38/215; A61K 47/6425; A61K 47/643; A61K 47/644; A61K 47/65; A61K 47/6889; A61P 35/00

(57) ABSTRACT

Provided herein are IFN polypeptide prodrugs comprising INF, a half-life extension element, an IFN blocking element and a protease cleavable linker. Also provided herein are pharmaceutical compositions thereof, as well as nucleic acids, recombinant expression vectors, host cells for making such polypeptide prodrugs. Also disclosed are methods of using the polypeptide prodrugs in the treatment of diseases, conditions and disorders.

### 1. RELATED APPLICATIONS

The present application claims the benefit of U.S. Provisional Application No. 63/234,284, filed on Aug. 18, 2021, which is hereby incorporated by reference in its entirety.

### 2. BACKGROUND

Interferons (“IFNs”) are a family of related signal proteins grouped in three major types, alpha, beta and gamma. Upon binding to specific receptors they lead to the activation of a signal transduction pathway that activates a broad range of genes, that are now known involved not only in antiviral but also in immunomodulatory and antiproliferative activities.

IFN's are a potent immune antagonist and has been considered a promising therapeutic agent for oncology. However, IFN's have shown to have a narrow therapeutic window because they are highly potent and have a short serum half-life. Consequently, therapeutic administration of IFN produce undesirable systemic effects and toxicities. This is exacerbated by the need to administer large quantities of cytokines (i.e., IFN) in order to achieve the desired levels of cytokine at the intended site of cytokine action (e.g., a tumor microenvironment). Unfortunately, due to the biology of cytokine and the inability to effectively target and control their activity, cytokines have not achieved the hoped for clinical advantages in the treatment in tumors.

Inducible IFN protein constructs have been described in International Application Nos. PCT/US2019/032320 and PCT/US2020/060624 to overcome the toxicity and short half-life problems that have limited clinical use of IFN in oncology. The previously described inducible IFN polypeptide constructs comprise a polypeptide chain containing IFN and a human serum albumin or an antigen binding polypeptide that binds human serum albumin that also is capable of extending the half-life.

### 3. SUMMARY

The disclosure relates to inducible IFN prodrugs that contain at least one polypeptide chain, and can contain two or more polypeptides, if desired. The inducible IFN prodrug comprises a IFN polypeptide, a blocking element, a protease cleavable linker, and a half-life extension element. Exemplary IFN's include IFN-alpha (e.g., human IFN-alpha1, human IFN-alpha2, human IFN-alpha4, human IFN-alpha5, human IFN-alpha6, human IFN-alpha7, human IFN-alpha8, human IFN-alpha 10, human IFN-alpha13, human IFN-alpha14, human IFN-alpha16, human IFN-alpha17, human IFN-alpha2), IFN-beta, IFN-kappa, or IFN-epsilon, and functional fragments or muteins of any of the foregoing. In particular, the IFN can be IFN alpha, IFN beta, IFN gamma, a mutein, or an active fragment of the foregoing. A preferred IFN is IFN alpha.

Inducible IFN prodrugs of this disclosure have attenuated IFN receptor agonist activity and the circulating half-life is extended. The inducible IFN receptor agonist activity is attenuated through the blocking element. The half-life extension element can also contribute to attenuation, for example through steric effects. The blocking element is capable of blocking all or some of the receptor agonist activity of the IFN by noncovalently binding to the IFN and/or sterically blocking receptor binding. Upon cleavage of the protease cleavable linker a form of the IFN is released that is active (e.g., more active than the IFN polypeptide prodrug). Typically, the released IFN is at least 10 × more active than the IFN polypeptide prodrug. Preferably, the released IFN is at least 20 ×, at least 30 ×, at least 50 ×, at least 100 ×, at least 200 ×, at least 300 ×, at least 500 ×, at least 1000 ×, at least about 10,000× or more active than the inducible IFN prodrug.

The form of cytokine that is released upon cleavage of the inducible cytokine prodrug typically has a short half-life, which is often substantially similar to the half-life of naturally occurring cytokine. Even though the half-life of the inducible cytokine prodrug is extended, toxicity is reduced or eliminated because the agonist activity of the circulating inducible cytokine prodrug is attenuated and active cytokine is targeted to the desired site of activity (e.g., tumor microenvironment).

The inducible IFN prodrug can comprise at least one of each of a IFN polypeptide [A], a IFN blocking element [D], a half-life extension element [H], and a protease-cleavable polypeptide linker [L]. The IFN polypeptide and the IFN blocking element or the half-life extension element can be operably linked by the protease-cleavable polypeptide linker and the inducible IFN prodrug has attenuated IFN receptor activating activity. The IFN receptor activating activity of the inducible IFN prodrug is at least about 10× less than the IFN receptor activating activity of the polypeptide that contains the IFN polypeptide that is produced by cleavage of the protease cleavable linker.

The inducible IFN prodrug of can have the formula:

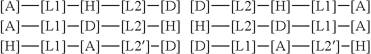

[A] is a IFN polypeptide, [D] is a blocking element, [H] is a half-life extension element, [L1] is a protease-cleavable polypeptide linker, [L2] is a polypeptide linker that is optionally protease-cleavable, and [L2′] is a protease-cleavable polypeptide linker.

The half-life extension element can comprises a serum albumin binding domain, a serum albumin, transferrin, or immunoglobulin Fc, or fragment thereof. The half-life extension element can also a blocking element.

The blocking element comprises a ligand-binding domain or fragment of a cognate receptor for the IFN, an antibody or antigen-binding fragment of an antibody that binds to the IFN polypeptide. The antibody or antigen-binding fragment can be a single domain antibody, a Fab, or a scFv that binds the IFN polypeptide. The cognate receptor for the IFN can be the IFN-α/β receptor. The cognate receptor for IFN can be the IFNAR1 chain or the IFNAR2 chain. The IFN blocking element inhibits activation of the IFN receptor by the inducible IFN prodrug.

Each protease-cleavable polypeptide linker independently comprises a sequence that is capable of being cleaved by a protease selected from the group consisting of a kallikrein, thrombin, chymase, carboxypeptidase A, cathepsin G, cathepsin L, an elastase, PR-3, granzyme M, a calpain, a matrix metalloproteinase (MMP), an ADAM, a FAP, a plasminogen activator, a cathepsin, a caspase, a tryptase, and a tumor cell surface protease. L2 can be a protease-cleavable polypeptide linker. L1 or L2 or both L1 and L2 can cleaved by two or more different proteases.

The cathepsin is cathepsin B, cathepsin C, cathepsin D, cathepsin E, cathepsin K, cathepsin L, cathepsin S, or cathepsin G. The matrix metalloprotease (MMP) can be MMP1, MMP2, MMP3, MMP8, MMP9, MMP10, MMP11, MMP12, MMP13, MMP14, MMP19, or MMP20.

The disclosure also relates to a nucleic acid encoding the inducible IFN prodrug disclosed herein. Also provided herein is a vector comprising the nucleic acid and a host cell comprising the vector.

The disclosure also relates to a pharmaceutical composition that contains the inducible IFN prodrug disclosed herein. Disclosed herein are methods of making the pharmaceutical composition comprising culturing the host cell under suitable conditions for expression and collection of the inducible IFN prodrug.

The disclosure also relates to therapeutic methods that include administering to a subject in need thereof an effective amount of a inducible IFN prodrug, nucleic acid that encodes the inducible IFN prodrug, vector or host cells that contain such a nucleic acid, and pharmaceutical compositions of any of the foregoing. Typically, the subject has, or is at risk of developing cancer, a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. The methods disclosed herein are particularly suitable for treating cancer. The inducible IFN prodrug can be administered intravenously.

### 4. BRIEF DESCRIPTION OF THE DRAWINGS

The drawings are not necessarily to scale or exhaustive. Instead, the emphasis is generally placed upon illustrating the principles of the inventions described herein. The accompanying drawings, which constitute a part of the specification, illustrate several embodiments consistent with the disclosure and, together with the description, serve to explain the principles of the disclosure. In the drawings:

FIGS. 1A-1C depicts a graph showing activity of IFN inducible polypeptide, WW0888, having an antibody blocking element in an HEKBlue IFN reporter assay (FIG. 1A), SDS-PAGE (FIG. 1B), and a SEC analysis (FIG. 1C). FIG. 1A depicts activation of the IFN-α/β pathway in a comparison of WW0888 to human IFNalpha (control). Squares depict activity of the uncut WW0888 polypeptide (intact) and diamonds depict the activity of the cut polypeptide (cleaved). Circles depict activity of the control (human IFNalpha). EC50 values for each are shown in the table. Analysis was performed based on quantification of Secreted Alkaline Phosphatase (SEAP) activity using the reagent QUANTI-Blue® (InvivoGen). Results confirm that the IFNalpha fusion protein was active and inducible. FIG. 1B shows results of protein cleavage assay. Fusion protein WW0888 was run on an SDS-PAGE gel in both cleaved and uncleaved form. As can be seen in the gel. FIG. 1C shows a graph from a SEC analysis of WW0888.

FIGS. 2A-2C depicts a graph showing activity of IFN inducible IFN prodrug, WW0889/890, having an antibody blocking element in an HEKBlue IFN reporter assay (FIG. 2A), SDS-PAGE (FIG. 2B), and a SEC analysis (FIG. 2C). FIG. 2A depicts activation of the IFN-α/β pathway in a comparison of WW0889/890 to human IFNalpha (control). Squares depict activity of the uncut WW0889/890 polypeptide (intact) and diamonds depict the activity of the cut polypeptide (cleaved). Circles depict activity of the control (human IFNalpha2b). EC50 values for each are shown in the table. Analysis was performed based on quantification of Secreted Alkaline Phosphatase (SEAP) activity using the reagent QUANTI-Blue® (InvivoGen). Results confirm that WW0889/890 was active and inducible. FIG. 2B shows results of protein cleavage assay. WW0889/890 was run on an SDS-PAGE gel in both cleaved and uncleaved form. As can be seen in the gel, cleavage was complete. FIG. 2C shows a graph from a SEC analysis of WW0889/890.

FIGS. 3A-3C depicts a graph showing activity of IFN inducible prodrug, WW0891/892, having an antibody blocking element in an HEKBlue IFN reporter assay (FIG. 3A), SDS-PAGE (FIG. 3B), and a SEC analysis (FIG. 3C). FIG. 3A depicts activation of the IFN-α/β pathway in a comparison of WW0891/892 to human IFNalpha2b (control). Squares depict activity of the uncut WW0891/892 polypeptide (intact) and diamonds depict the activity of the cut polypeptide (cleaved). Circles depict activity of the control (human IFNalpha). EC50 values for each are shown in the table. Analysis was performed based on quantification of Secreted Alkaline Phosphatase (SEAP) activity using the reagent QUANTI-Blue® (InvivoGen). Results confirm that WW0891/892 was active and inducible. FIG. 3B shows results of protein cleavage assay. WW0891/892 was run on an SDS-PAGE gel in both cleaved and uncleaved form. As can be seen in the gel, cleavage was complete. FIG. 3C shows a graph from a SEC analysis of WW0891/892.

FIGS. 4A-4C depicts a graph showing activity of IFN inducible polypeptide, WW0894, having an IFNa2b receptor 1(R1) blocking element in an HEKBlue IFN reporter assay (FIG. 4A), SDS-PAGE (FIG. 4B), and a SEC analysis (FIG. 4C). FIG. 4A depicts activation of the IFN-α/β pathway in a comparison of WW0894 to human IFNalpha2b (control). Squares depict activity of the uncut WW0894 polypeptide (intact) and triangles depict the activity of the cut polypeptide (cleaved). Circles depict activity of the control (human IFNalpha). EC50 values for each are shown in the table. Analysis was performed based on quantification of Secreted Alkaline Phosphatase (SEAP) activity using the reagent QUANTI-Blue® (InvivoGen). Results confirm that WW0894 was active and inducible. FIG. 4B shows results of protein cleavage assay. Fusion protein WW0894 was run on an SDS-PAGE gel in both cleaved and uncleaved form. As can be seen in the gel, cleavage was complete. FIG. 4C shows a graph from a SEC analysis of WW0894.

FIGS. 5A-5C depicts a graph showing activity of IFN inducible polypeptide, WW0893, having an IFNa2b receptor 2, (R2) blocking element in an HEKBlue IFN reporter assay (FIG. 5A), SDS-PAGE (FIG. 5B), and a SEC analysis (FIG. 5C). FIG. 5A depicts activation of the IFN-α/β pathway in a comparison of WW0893 to human IFNalpha (control). Squares depict activity of the uncut WW0893 polypeptide (intact) and triangles depict the activity of the cut polypeptide (cleaved). Circles depict activity of the control (human IFNalpha). EC50 values for each are shown in the table. Analysis was performed based on quantification of Secreted Alkaline Phosphatase (SEAP) activity using the reagent QUANTI-Blue® (InvivoGen). Results confirm that WW0893 was active and inducible. FIG. 5B shows results of protein cleavage assay. Fusion protein WW0893 was run on an SDS-PAGE gel in both cleaved and uncleaved form. As can be seen in the gel, cleavage was complete. FIG. 5C shows a graph from a SEC analysis of WW0893.

FIGS. 6A-6C depicts a graph showing activity of IFN inducible polypeptide, WW0895, having an IFNa2b receptor 1, (R1) blocking element in an HEKBlue IFN reporter assay (FIG. 6A), SDS-PAGE (FIG. 6B), and a SEC analysis (FIG. 6C). FIG. 6A depicts activation of the IFN-α/β pathway in a comparison of WW0895 to human IFNalpha (control). Squares depict activity of the uncut WW0895 polypeptide (intact) and triangles depict the activity of the cut polypeptide (cleaved). Circles depict activity of the control (human IFNalpha). EC50 values for each are shown in the table. Analysis was performed based on quantification of Secreted Alkaline Phosphatase (SEAP) activity using the reagent QUANTI-Blue® (InvivoGen). Results confirm that WW0895 was active and inducible. FIG. 6B shows results of protein cleavage assay. Fusion protein WW0895 was run on an SDS-PAGE gel in both cleaved and uncleaved form. As can be seen in the gel, cleavage was complete. FIG. 6C shows a graph from a SEC analysis of WW0895.

FIGS. 7A-7C depicts a graph showing activity of IFN inducible polypeptide, WW0896, having an IFNa2b receptor 1 and 2 (R1 and R2) blocking elements in an HEKBlue IFN reporter assay (FIG. 7A), SDS-PAGE (FIG. 7B), and a SEC analysis (FIG. 7C). FIG. 7A depicts activation of the IFN-α/β pathway in a comparison of WW0896 to human IFNalpha2b (control). Squares depict activity of the uncut WW0896 polypeptide (intact) and triangles depict the activity of the cut polypeptide (cleaved). Circles depict activity of the control (human IFNalpha2b). EC50 values for each are shown in the table. Analysis was performed based on quantification of Secreted Alkaline Phosphatase (SEAP) activity using the reagent QUANTI-Blue® (InvivoGen). Results confirm that WW0896 was active and inducible. FIG. 7B shows results of protein cleavage assay. Fusion protein WW0896 was run on an SDS-PAGE gel in both cleaved and uncleaved form. As can be seen in the gel, cleavage was complete. FIG. 7C shows a graph from a SEC analysis of WW0896.

FIGS. 8A-8C depicts a graph showing activity of IFN inducible prodrug, WW0897, having an IFNa2b receptor 1 and 2 (R1 and R2) blocking elements in an HEKBlue IFN reporter assay (FIG. 8A), SDS-PAGE (FIG. 8B), and a SEC analysis (FIG. 8C). FIG. 8A depicts activation of the IFN-α/β pathway in a comparison of WW0897 to human IFNalpha2b (control). Squares depict activity of the uncut WW0897 polypeptide (intact) and triangles depict the activity of the cut polypeptide (cleaved). Circles depict activity of the control (human IFNalpha2b). EC50 values for each are shown in the table. Analysis was performed based on quantification of Secreted Alkaline Phosphatase (SEAP) activity using the reagent QUANTI-Blue® (InvivoGen). Results confirm that WW0897 was active and inducible. FIG. 8B shows results of protein cleavage assay. Fusion protein WW0897 was run on an SDS-PAGE gel in both cleaved and uncleaved form. As can be seen in the gel, cleavage was complete. FIG. 8C shows a graph from a SEC analysis of WW0897.

FIGS. 9A-9C depicts a graph showing activity of IFN inducible prodrug, WW0898, having an IFNa2b receptor 1 and 2 (R1 and R2) blocking elements in an HEKBlue IFN reporter assay (FIG. 9A) SDS-PAGE (FIG. 9B), and a SEC analysis (FIG. 9C). FIG. 9A depicts activation of the IFN-α/β pathway in a comparison of WW0898 to human IFNalpha2b (control). Squares depict activity of the uncut WW0898 polypeptide (intact) and triangles depict the activity of the cut polypeptide (cleaved). Circles depict activity of the control (human IFNalpha2b). EC50 values for each are shown in the table. Analysis was performed based on quantification of Secreted Alkaline Phosphatase (SEAP) activity using the reagent QUANTI-Blue® (InvivoGen). Results confirm that WW0898 was active and inducible. FIG. 9B shows results of protein cleavage assay. Fusion protein WW0898 was run on an SDS-PAGE gel in both cleaved and uncleaved form. As can be seen in the gel, cleavage was complete. FIG. 9C shows a graph from a SEC analysis of WW0898.

FIG. 10 is a graph showing average MC38 tumor volumes (mm³) over time after dosing in mice treated with vehicle (circles) and inducible IFN prodrug WW00901 at 75 μg (squares), 300 μg (triangles), 600 μg (star).

FIGS. 11A-11D are graphs of MC38 tumor volume in individual mice treated with vehicle (FIG. 11A), inducible IFN prodrug WW00901 at 75 μg (FIG. 11B), inducible IFN prodrug WW00901 at 300 μg (FIG. 11C), and inducible IFN prodrug WW00901 at 600 μg (FIG. 11D).

FIG. 12 is a graph showing average body weights of mice treated with vehicle (circles) and inducible IFN prodrug WW00901 at 75 μg (squares), 300 μg (triangles), 600 μg (stars).

FIGS. 13A-13G are a series of graphs showing activity of IFN inducible prodrugs in the B16-Blue IFN-α/β reporter assay. FIGS. 13A-13G depict activation of the IFN-α/β pathway in a comparison of inducible IFN prodrug to mouse INFα1 (control). Squares depict activity of the uncut inducible IFN prodrug (intact), and triangles (or diamonds in the case of FIG. 13A) depict the activity of the cut inducible IFN prodrug (cleaved). Circles (filled and open) depict activity of the control (mouse IFNα1). Each inducible IFN prodrug was run on an SDS-PAGE gel in both cleaved and uncleaved form. As can be seen in the gel, cleavage was complete.

FIGS. 14A, 14C, 14E, 14G, 14I, 14L, 14M depict graphs showing activity of IFN inducible prodrugs in an HEKBlue IFN reporter assay. The activity of the uncut IFN inducible prodrug (intact, triangles in FIGS. 14A and 14B, and squares in FIGS. 14E, 14G, 14I and 14L) and the activity of the cut IFN inducible prodrug (cleaved, squares in FIGS. 14A and 14B, and triangles in FIGS. 14E, 14G, 14I and 14L) is shown. Circles and inverted triangles depict activity of the control (human IFNalpha2b). EC50 values for each are shown in the table (N.D.=not determined). Analysis was performed based on quantification of Secreted Alkaline Phosphatase (SEAP) activity using the reagent QUANTI-Blue® (InvivoGen). Results confirm that the inducible IFN prodrugs are active and inducible. FIGS. 14B, 14D, 14F, 14H, 14J, and 14K show the results of protein cleavage assay. IFN inducible prodrugs were run on an SDS-PAGE gel in both cleaved and uncleaved form. As can be seen in the gel, cleavage was complete.

### 5. DETAILED DESCRIPTION

This disclosure relates to inducible IFN polypeptides and to methods of using and compositions that contain the inducible IFN polypeptides. The inducible IFN polypeptides overcome the toxicity and short half-life problems that have severely limited the clinical use of cytokines in oncology.

The inducible IFN disclosed herein comprises one or more polypeptide chains and includes an IFN polypeptide (e.g., IFN-alpha, IFN-beta, or IFN-gamma) that has receptor agonist activity of native IFN, including binding to and activating signally through a IFN receptor (e.g., IFN-α/β), a half-life extension element, an IFN blocking element, and a protease cleavable linker. The inducible IFN, in the form of a single polypeptide chain or a complex of two or more polypeptide chains, has attenuated IFN receptor activity, e.g., due to the action of the blocking element, and the circulating half-life is extended.

The inducible IFN contain a protease cleavable linker that includes one or more protease cleave sites, which are cleaved by proteases that are associated with, and are typically enriched or selectively present in, the tumor microenvironment. Thus, the inducible IFNs are preferentially (or selectively) and efficiently cleaved in the tumor microenvironment to release active IFN, and to limit IFN activity substantially to the tumor microenvironment. The IFN that is released upon cleavage has a short half-life, which is substantially similar to the half-life of naturally occurring IFN, further restricting IFN activity to the tumor microenvironment. Even though the half-life of the inducible IFN prodrug is extended, toxicity is dramatically reduced or eliminated because the circulating prodrug has attenuated IFN activity, and active IFN is targeted to the tumor microenvironment.

This disclosure further relates to pharmaceutical compositions that contain the inducible IFNs, as well as nucleic acids that encode the polypeptides, and recombinant expression vectors and host cells for making such inducible IFNs. Also provided herein are methods of using the disclosed inducible IFNs in the treatment of diseases, conditions, and disorders.

### A. Inducible Interferon Prodrug

The disclosure relates to inducible IFN polypeptide prodrugs that contain at least one polypeptide chain, and can contain two or more polypeptide chains, if desired. The inducible IFN prodrugs comprises a IFN or a mutein thereof, a half-life extension element, an IFN blocking element, and a protease cleavable linker. The IFN can be a Type I, Type II, or Type III IFN. Type I IFN's that can be suitable include IFN-alpha (e.g., human IFN-alpha1, human IFN-alpha2, human IFN-alpha4, human IFN-alpha5, human IFN-alpha6, human IFN-alpha7, human IFN-alpha8, human IFN-alpha10, human IFN-alpha13, human IFN-alpha 14, human IFN-alpha16, human IFN-alpha17, human IFN-alpha2), IFN-beta, IFN-kappa, or IFN-epsilon. IFN-alpha and IFN-beta are preferred. A type II IFN that is suitable for the inducible IFN polypeptide prodrugs disclosed herein is IFN-gamma.

The inducible IFNs of this disclosure have attenuated IFN receptor agonist activity and the circulating half-life is extended. The IFN receptor agonist activity is attenuated through the blocking element. The half-life extension element can also contribute to attenuation, for example through steric effects. The half-life extension element can also act as a blocking element that is capable of blocking all or some of the receptor agonist activity of IFN. For instance, the half-life extension element can contribute to blocking when the half-life extension element is adjacent to the IFN polypeptide.

The blocking element is capable of blocking all or some of the receptor agonist activity of IFN by noncovalently binding to the IFN (e.g., to IFN-alpha or IFN-beta) and/or sterically blocking receptor binding. Upon cleavage of the protease cleavable linker a form of IFN is released that is active (e.g., more active than the inducible IFN prodrug). Typically, the released IFN is at least 10 × more active than the inducible IFN prodrug. Preferably, the released IFN is at least 20 ×, at least 30 ×, at least 50 ×, at least 100 ×, at least 200 ×, at least 300 ×, at least 500 ×, at least 1000 ×, at least about 10,000× or more active than the inducible IFN prodrug.

The form of IFN that is released upon cleavage of the inducible IFN prodrug typically has a short half-life, which is often substantially similar to the half-life of naturally occurring IFN. Even though the half-life of the inducible IFN prodrug is extended, toxicity is reduced or eliminated because the agonist activity of the circulating inducible IFN prodrug is attenuated and active IFN is targeted to the desired site of activity (e.g., tumor microenvironment).

It will be appreciated by those skilled in the art, that the number of polypeptide chains, and the location of the elements, the half-life extension element, the protease cleavable linker(s), and the blocking element (and components of such elements, such as a VH or VL domain) on the polypeptide chains can vary and is often a matter of design preference. All such variations are encompassed by this disclosure.

The inducible IFN prodrug can comprise a single polypeptide chain. Typically, the single polypeptide complex comprises a IFN polypeptide or a mutein thereof [A], a blocking element [D], a half-life extension element [H], and a protease cleavable linker [L]. The IFN [A] polypeptide can be operably linked to the blocking element, the half-life extension element or both the blocking element, the half-life extension element by a protease cleavable linker. The protease cleavable linker can comprise the sequence GPAGLYAQ (SEQ ID NO: 195) or ALFKSSFP (SEQ ID NO: 198).

The single polypeptide complex can comprise a IFN polypeptide [A], a blocking element [D], a half-life extension element [H], and a protease cleavable linker having the amino acid sequence GPAGLYAQ (SEQ ID NO: 195) or ALFKSSFP (SEQ ID NO: 198) [L]. The IFN [A] polypeptide can be operably linked to the blocking element, the half-life extension element or both the blocking element, the half-life extension element by a protease cleavable linker.

The single polypeptide complex can comprise a IFN polypeptide [A], a blocking element [D], a half-life extension element [H], and a protease cleavable linker having the amino acid sequence GPAGLYAQ (SEQ ID NO: 195) [L]. The IFN [A] polypeptide can be operably linked to the blocking element, the half-life extension element or both the blocking element, the half-life extension element by a protease cleavable linker.

The single polypeptide complex can comprise a IFN polypeptide [A], a blocking element [D], a half-life extension element [H], and a protease cleavable linker having the amino acid sequence ALFKSSFP (SEQ ID NO: 198) [L]. The IFN [A] polypeptide can be operably linked to the blocking element, the half-life extension element or both the blocking element, the half-life extension element by a protease cleavable linker.

The IFN polypeptide and the blocking element and the half-life extension element are operably linked by the protease-cleavable polypeptide. For example, the polypeptide can be of any of Formulas (I)-(IX).

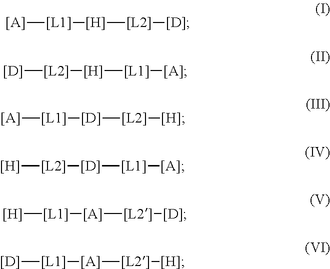

In Formulas (I)-(IX), [A] is a IFN polypeptide, [D] is a IFN blocking element (e.g., extracellular portion of the INFalpha receptor 1 (IFNAR1) or IFNalpha receptor 2 (IFNAR2) or an antibody or antigen-binding fragment), [D′] is either the INFalpha receptor 1 (IFNAR1) or the IFNalpha receptor 2 (IFNAR2) that is not present in [D], [H] is a half-life extension element, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can have the same or different amino acid sequence and or protease-cleavage site (when L2 is protease-cleavable) as desired. [H] can also optionally provide blocking. The protease cleavable linker can comprise the sequence GPAGLYAQ (SEQ ID NO: 195) or ALFKSSFP (SEQ ID NO: 198).

While the inducible IFN prodrugs disclosed herein preferably contain one half-life extension element and one blocking element, such elements can contain two or more components that are present on the same polypeptide chain or on different polypeptide chains. Illustrative of this, and as disclosed and exemplified herein, components of the blocking element can be present on separate polypeptide chains. For example, a first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the peptide complex to form an antigen-binding site, such as a Fab that binds IFN (e.g., IFNalpha, IFNbeta) and attenuates IFN activity.

For example, the inducible IFN prodrug can have a first polypeptide of Formulas (X-XI). Formula X: [D]-[L]-[A]-[L2]-[H] or Formula XI: [H]-[L]-[A]-[L2]-[D]. In Formulas (X)-(XI), [A] is a IFN polypeptide, [D] is a IFN antibody heavy chain Fab fragment (VH+CH1) or heavy chain variable domain (VH), [H] is a half-life extension element, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can be have the same or different amino acid sequence and or protease-cleavage site (when L2 is protease-cleavable) as desired. The inducible IFN prodrug can have a second polypeptide antibody light chain (VL+CL) or light chain variable domain (VL) that is complementary to the VH+CH1 or VH. The protease cleavable linker can comprise the sequence GPAGLYAQ (SEQ ID NO: 195) or ALFKSSFP (SEQ ID NO: 198).

The inducible IFN prodrugs can comprise or consist of the amino acid sequence of SEQ ID NOs: 1, 6-11, 12-16, 18-23, or 30-35. For example, the inducible IFN prodrug can comprise the amino acid sequence of SEQ ID NO: 1. For example, the inducible IFN prodrug can comprise the amino acid sequence of SEQ ID NO: 6. For example, the inducible IFN prodrug can comprise the amino acid sequence of SEQ ID NO: 7. For example, the inducible IFN prodrug can comprise the amino acid sequence of SEQ ID NO: 8. For example, the inducible IFN prodrug can comprise the amino acid sequence of SEQ ID NO: 9. For example the inducible IFN prodrug can comprise the amino acid sequence of SEQ ID NO: 10. For example, the inducible IFN prodrug can comprise the amino acid sequence of SEQ ID NO: 11. For example, the inducible IFN prodrug can comprise the amino acid sequence of SEQ ID NO: 12. For example, the inducible IFN prodrug can comprise the amino acid sequence of SEQ ID NO: 13. For example, the inducible IFN prodrug can comprise the amino acid sequence of SEQ ID NO: 14. For example, the inducible IFN prodrug can comprise the amino acid sequence of SEQ ID NO: 15. For example, the inducible IFN prodrug can comprise the amino acid sequence of SEQ ID NO: 16. For example, the inducible IFN prodrug can comprise the amino acid sequence of SEQ ID NO: 18. For example, the inducible IFN prodrug can comprise the amino acid sequence of SEQ ID NO: 19. For example, the inducible IFN prodrug can comprise the amino acid sequence of SEQ ID NO: 20. For example, the inducible IFN prodrug can comprise the amino acid sequence of SEQ ID NO: 21. For example, the inducible IFN prodrug can comprise the amino acid sequence of SEQ ID NO: 22. For example, the inducible IFN prodrug can comprise the amino acid sequence of SEQ ID NO: 23. For example, the inducible IFN prodrug can comprise the amino acid sequence of SEQ ID NO: 30. For example, the inducible IFN prodrug can comprise the amino acid sequence of SEQ ID NO: 31. For example, the inducible IFN prodrug can comprise the amino acid sequence of SEQ ID NO: 32. For example, the inducible IFN prodrug can comprise the amino acid sequence of SEQ ID NO: 33. For example, the inducible IFN prodrug can comprise the amino acid sequence of SEQ ID NO: 34. For example, the inducible IFN prodrug can comprise the amino acid sequence of SEQ ID NO: 35.

In embodiments, the inducible IFN cytokine prodrug can contain a first polypeptide that is bonded covalently or non-covalently to a second polypeptide chain. The second polypeptide chain can contain an antibody VL-CL that comprises or consists of the amino acid sequence of SEQ ID NO: 3 or SEQ ID NO: 5. Such a second polypeptide can bond with a complimentary VH-CH1 polypeptide contained within the fusion protein, e.g., as contained within SEQ ID NO: 2 or SEQ ID NO: 4. For example, the inducible IFN cytokine prodrug can comprise or consist the amino acid sequence of SEQ ID NO: 2 and the second polypeptide chain can comprise or consist the amino acid sequence of SEQ ID NO: 3. For example, the inducible IFN cytokine prodrug can comprise or consist the amino acid sequence of SEQ ID NO: 4 and the second polypeptide chain can comprise or consist the amino acid sequence of SEQ ID NO: 5. The second polypeptide chain can contain an antibody VH-CH1 that comprises or consists of the amino acid sequence of SEQ ID NO: 17. Such a second polypeptide can bond with complimentary VL-CL polypeptide contained within the first polypeptide chain, e.g., as contained within SEQ ID NO: 24, 25 or 28. For example, the inducible IFN cytokine prodrug can include a) a first polypeptide chain that comprises or consist of the amino acid sequence of SEQ ID NO: 24 and b) a second polypeptide chain that comprises or consists of the amino acid sequence of SEQ ID NO: 17. For example, the inducible IFN cytokine prodrug can include a) a first polypeptide chain that comprises or consists the amino acid sequence of SEQ ID NO: 25 and b) a second polypeptide chain that comprises or consists of the amino acid sequence of SEQ ID NO: 17. For example, the inducible IFN cytokine prodrug can include a) a first polypeptide chain that comprises or consists the amino acid sequence of SEQ ID NO: 28 and b) a second polypeptide chain that comprises or consists of the amino acid sequence of SEQ ID NO: 17.

In embodiments, the inducible IFN cytokine prodrug can comprise a first polypeptide chain that comprises an IFN polypeptide and an antibody light chain (VL+CL) or light chain variable domain (VL) and a second polypeptide can include a half-life extension element and an antibody heavy chain Fab fragment (VH+CH1) or heavy chain variable domain (VH) that is complementary to the VL+CL or VL on the first polypeptide. For example, the inducible IFN cytokine prodrug can include a) a first polypeptide that comprises or consists of the amino acid sequence of SEQ ID NO: 26, and b) a second polypeptide chain that comprises or consists of the amino acid sequence of SEQ ID NO. 27. For example, the inducible IFN cytokine prodrug can include a) a first polypeptide that comprises or consists of the amino acid sequence of SEQ ID NO: 26, and b) a second polypeptide chain that comprises or consists of the amino acid sequence of SEQ ID NO. 29.

In embodiments, the inducible IFN cytokine prodrug can comprise a first polypeptide chain that comprises an IFN polypeptide and an antibody heavy chain Fab fragment (VH+CH1) or heavy chain variable domain (VH) and a second polypeptide can include a half-life extension element and an antibody light chain (VL+CL) or light chain variable domain (VL) that is complementary to the VH+CH1 or VH on the first polypeptide.

### B. Half-Life Extension Element

The half-life extension element, increases the in vivo half-life and provides altered pharmacodynamics and pharmacokinetics of the inducible IFN prodrugs. Without being bound by theory, the half-life extension element alters pharmacodynamics properties including alteration of tissue distribution, penetration, and diffusion of the inducible IFN prodrug. In some embodiments, the half-life extension element can improve tissue targeting, tissue penetration, diffusion within the tissue, and enhanced efficacy as compared with a protein without a half-life extension element. Without being bound by theory, an exemplary way to improve the pharmacokinetics of a polypeptide is by expression of an element in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins, or fragments retain FcRn binding and are routinely linked to other polypeptides to extend their serum half-life. HSA may also be directly bound to the pharmaceutical compositions or bound via a short linker. Fragments of HSA may also be used. HSA and fragments thereof can function as both a blocking element and a half-life extension element. Human IgGs and Fc fragments can also carry out a similar function.

The serum half-life extension element can also be an antigen-binding polypeptide that binds to a protein with a long serum half-life such as serum albumin, transferrin and the like. Examples of such polypeptides include antibodies and fragments thereof including, a polyclonal antibody, a recombinant antibody, a human antibody, a humanized antibody a single chain variable fragment (scFv), an antigen binding fragment (Fab), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens. The antibodies and fragments thereof can function as both a blocking element and a half-life extension element.

The half-life extension element can also function as both a blocking element and a half-life extension element. For instance, the half-life extension element (e.g., anti-HSA) can function as a blocking element when adjacent to the IFN polypeptide.

The half-life extension element as provided herein is preferably a human serum albumin (HSA) binding domain, and antigen binding polypeptide that binds human serum albumin or an immunoglobulin Fc or fragment thereof.

The half-life extension element of a inducible IFN prodrug extends the half-life of the inducible IFN prodrug by at least about two days, about three days, about four days, about five days, about six days, about seven days, about eight days, about nine days, about 10 days or more.

### C. Blocking Element

The blocking element can be any element that binds to IFN and/or inhibits the ability of the IFN polypeptide to bind and activate its receptor. The blocking element can inhibit the ability of the IFN to bind and/or activate its receptor e.g., by sterically blocking and/or by noncovalently binding to the inducible IFN prodrug. Some blocking elements disclosed herein can bind to IFN (e.g., IFN-alpha (e.g., human IFN-alpha1, human IFN-alpha2, human IFN-alpha4, human IFN-alpha5, human IFN-alpha6, human IFN-alpha7, human IFN-alpha8, human IFN-alpha10, human IFN-alpha13, human IFN-alpha14, human IFN-alpha16, human IFN-alpha17, human IFN-alpha2) IFN-beta, IFN-gamma).

Examples of suitable blocking elements include the full length or an IFN-binding fragment or mutein of the cognate receptor of an IFN. The cognate receptor for IFN can be the IFNGR receptor or a portion thereof. For instance, when the interferon polypeptide is an IFNalpha, such as INFalpha2a, the blocking element can be the extracellular portion of the INFalpha receptor 1 (IFNAR1) or interferon binding portion or mutein thereof, or the extracellular portion of the IFNalpha receptor 2 (IFNAR2) or interferon binding portion or mutein thereof. When the interferon polypeptide is IFNgamma, the blocking element can be the extracelluar portion of the IFNgamma receptor 1 (IFNGR1) or interferon binding portion or mutein thereof, or the extracellular portion of the IFNgamma receptor 2 (IFNGR2) or interferon binding portion or mutein thereof.

Antibodies and antigen-binding fragments thereof including, an antigen-binding fragment (Fab), a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like that bind IFN can also be used. Other suitable antigen-binding domain that bind IFN can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of suitable blocking polypeptides include polypeptides that sterically inhibit or block binding of IFN to its cognate receptor. Advantageously, such moieties can also function as half-life extending elements. For example, a peptide that is modified by conjugation to a water-soluble polymer, such as PEG, can sterically inhibit or prevent binding of the cytokine to its receptor. Polypeptides, or fragments thereof, that have long serum half-lives can also be used, such as serum albumin (human serum albumin), immunoglobulin Fc, transferrin and the like, as well as fragments and muteins of such polypeptides.

IFN blocking elements that are particularly suitable are single chain variable fragments (scFv) or Fab fragments.

Also disclosed herein is an inducible IFN polypeptide that contains a blocking element having specificity for IFN and further contains a half-life extension element.

The blocking element can contain two or more components that are present on the same polypeptide chain or on separate polypeptide chains. A first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the peptide complex to form an antigen-binding site, such as a Fab that binds IFN (e.g., IFNalpha, IFNbeta) and attenuates IFN activity.

### D. Protease Cleavable Linker

As disclosed herein, the inducible IFN prodrug comprises one or more linker sequences. A linker sequence serves to provide flexibility between the polypeptides, such that, for example, the blocking element is capable of inhibiting the activity of IFN. The linker can be located between the IFN subunit, the half-life extension element, and/or the blocking element. As described herein the inducible IFN prodrug comprises a protease cleavable linker. The protease cleavable linker can comprise one or more cleavage sites for one or more desired protease. Preferably, the desired protease is enriched or selectively expressed at the desired target site of IFN activity (e.g., the tumor microenvironment). Thus, the inducible IFN prodrug is preferentially or selectively cleaved at the target site of desired IFN activity.

Suitable linkers are typically less than about 100 amino acids. Such linkers can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domain. In a preferred embodiment, the linker is cleavable by a cleaving agent, e.g., an enzyme. Preferably, the linker comprises a protease cleavage site. In some cases, the linker comprises one or more cleavage sites. The linker can comprise a single protease cleavage site. The linker can also comprise 2 or more protease cleavage sites. For example, 2 cleavage sites, 3 cleavage sites, 4, cleavage sites, 5 cleavage sites, or more. In cases the linker comprises 2 or more protease cleavage sites, the cleavage sites can be cleaved by the same protease or different proteases. A linker comprising two or more cleavage sites is referred to as a “tandem linker.” The two or more cleavage sites can be arranged in any desired orientation, including, but not limited tom one cleavage site adjacent to another cleavage site, one cleavage site overlapping another cleavage site, or one cleavage site following by another cleavage site with intervening amino acids between the two cleavage sites.

Of particular interest in the present invention are disease specific protease-cleavable linkers. Also preferred are protease-cleavable linkers that are preferentially cleaved at a desired location in the body, such as the tumor microenvironment, relative to the peripheral circulation. For example, the rate at which the protease-cleavable linker is cleaved in the tumor microenvironment can be at least about 10 times, at least about 100 times, at least about 1000 times or at least about 10,000 times faster in the desired location in the body, e.g., the tumor microenvironment, in comparison to in the peripheral circulation (e.g., in plasma).

Proteases known to be associated with diseased cells or tissues include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin G, Cathepsin S, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, MMP19, MMP20, urokinase plasminogen activator (uPA), enterokinase, prostate-specific antigen (PSA, hK3), interleukin-1β converting enzyme, thrombin, FAP (FAPα), dipeptidyl peptidase, meprins, granzymes and dipeptidyl peptidase IV (DPPIV/CD26). Proteases capable of cleaving linker amino acid sequences (which can be encoded by the chimeric nucleic acid sequences provided herein) can, for example, be selected from the group consisting of a prostate specific antigen (PSA), a matrix metalloproteinase (MMP), an A Disintigrin and a Metalloproteinase (ADAM), a plasminogen activator, a cathepsin, a caspase, a tumor cell surface protease, and an elastase. The MMP can, for example, be matrix metalloproteinase 2 (MMP2), matrix metalloproteinase 9 (MMP9), matrix metalloproteinase 14 (MMP14), matrix metalloproteinase 19 (MMP19), or matrix metalloproteinase 20 (MMP20). In addition, or alternatively, the linker can be cleaved by a cathepsin, such as, Cathepsin B, Cathepsin C, Cathepsin D, Cathepsin S, Cathepsin E, Cathepsin G, Cathepsin K and/or Cathepsin L. Preferably, the linker can be cleaved by MMP14 or Cathepsin L.

Proteases useful for cleavage of linkers and for use in the IFN polypeptide prodrug disclosed herein are presented in Table 1, and exemplary proteases and their cleavage site are presented in Table 2.

TABLE 1
Proteases relevant to inflammation and cancer

| | | |
|---|---|---|
| Protease | Specificity | Other aspects |

Secreted by killer T cells:

| | | |
|---|---|---|
| Granzyme B (grB) | Cleaves after Asp residues | Type of serine protease; strongly implicated |
| | (asp-ase) | in inducing perforin-dependent target cell |
| | | apoptosis |
| Granzyme A (grA) | trypsin-like, cleaves after | Type of serine protease; |
| | basic residues | |
| Granzyme H (grH) | Unknown substrate | Type of serine protease; |
| | specificity | Other granzymes are also secreted by killer |
| | | T cells, but not all are present in humans |
| Caspase-8 | Cleaves after Asp residues | Type of cysteine protease; plays essential |
| | | role in TCR-induced cellular expansion- |
| | | exact molecular role unclear |
| Mucosa-associated | Cleaves after arginine | Type of cysteine protease; likely acts both |
| lymphoid tissue (MALT1) | residues | as a scaffold and proteolytically active |
| | | enzyme in the CBM-dependent signaling |
| | | pathway |
| Tryptase | Targets: angiotensin I, | Type of mast cell-specific serine protease; |
| | fibrinogen, prourokinase, | trypsin-like; resistant to inhibition by |
| | TGFβ; preferentially | macromolecular protease inhibitors |
| | cleaves proteins after | expressed in mammals due to their |
| | lysine or arginine residues | tetrameric structure, with all sites facing |
| | | narrow central pore; also associated with |
| | | inflammation |

Associated with inflammation:

| | | |
|---|---|---|
| Thrombin | Targets: FGF-2, | Type of serine protease; modulates activity |
| | HB-EGF, Osteo-pontin, | of vascular growth factors, chemokines and |
| | PDGF, VEGF | extracellular proteins; strengthens VEGF- |
| | | induced proliferation; induces cell |
| | | migration; angiogenic factor; regulates |
| | | hemostasis |
| Chymase | Exhibit chymotrypsin-like | Type of mast cell-specific serine protease |
| | specificity, cleaving | |
| | proteins after aromatic | |
| | amino acid residues | |
| Carboxypeptidase A (MC- | Cleaves amino acid | Type of zinc-dependent metalloproteinase |
| CPA) | residues from C-terminal | |
| | end of peptides and | |
| | proteins | |
| Kallikreins | Targets: high molecular | Type of serine protease; modulate relaxation |
| | weight | response; contribute to inflammatory |
| | kininogen, pro-urokinase | response; fibrin degradation |
| Elastase | Targets: E-cadherin, GM- | Type of neutrophil serine protease; degrades |
| | CSF, IL-1, IL-2, IL-6, IL8, | ECM components; regulates inflammatory |
| | p38^{MAPK}, TNFα, VE- | response; activates pro-apoptotic signaling |
| | cadherin | |
| Cathepsin G | Targets: EGF, ENA-78, | Type of serine protease; degrades ECM |
| | IL-8, MCP-1, MMP-2, | components; chemo-attractant of |
| | MT1-MMP, | leukocytes; regulates inflammatory |
| | PAI-1, RANTES, TGFβ, | response; promotes apoptosis |
| | TNFα | |
| PR-3 | Targets: ENA-78, IL-8, IL- | Type of serine protease; promotes |
| | 18, JNK, p38^{MAPK}, TNFα | inflammatory response; activates pro- |
| | | apoptotic signaling |
| Granzyme M (grM) | Cleaves after Met and | Type of serine protease; only expressed in |
| | other long, unbranched | NK cells |
| | hydrophobic residues | |
| Calpains | Cleave between Arg and | Family of cysteine proteases; calcium- |
| | Gly | dependent; activation is involved in the |
| | | process of numerous inflammation- |
| | | associated diseases |

TABLE 2
Exemplary Proteases and Protease Recognition Sequences

| Protease | Cleavage Domain Sequence | SEQ ID NO: |
|---|---|---|
| MMP7 | KRALGLPG | 375 |
| MMP7 | (DE)₈RPLALWRS(DR)₈ | 376 |
| MMP9 | PR(S/T)(L/I)(S/T) | |
| MMP9 | LEATA | 378 |
| MMP11 | GGAANLVRGG | 379 |
| MMP14 | SGRIGFLRTA | 380 |
| MMP | PLGLAG | 381 |
| MMP | PLGLAX | 382 |
| MMP | PLGC(me)AG | 383 |
| MMP | ESPAYYTA | 384 |
| MMP | RLQLKL | 385 |
| MMP | RLQLKAC | 386 |
| MMP2, MMP9, MMP14 | EP(Cit)G(Hof)YL | 387 |
| Urokinase plasminogen activator (uPA) | SGRSA | 388 |
| Urokinase plasminogen activator (uPA) | DAFK | 389 |
| Urokinase plasminogen activator (uPA) | GGGRR | 390 |
| Lysosomal Enzyme | GFLG | 391 |
| Lysosomal Enzyme | ALAL | 392 |
| Lysosomal Enzyme | FK | |
| Cathepsin B | NLL | |
| Cathepsin D | PIC(Et)FF | 395 |
| Cathepsin K | GGPRGLPG | 396 |
| Prostate Specific Antigen | HSSKLQ | 397 |
| Prostate Specific Antigen | HSSKLQL | 398 |
| Prostate Specific Antigen | HSSKLQEDA | 399 |
| Herpes Simplex Virus Protease | LVLASSSFGY | 400 |
| HIV Protease | GVSQNYPIVG | 401 |
| CMV Protease | GVVQASCRLA | 402 |
| Thrombin | F(Pip)RS | |
| Thrombin | DPRSFL | 404 |
| Thrombin | PPRSFL | 405 |
| Caspase-3 | DEVD | 406 |
| Caspase-3 | DEVDP | 407 |
| Caspase-3 | KGSGDVEG | 408 |
| Interleukin 1ß converting enzyme | GWEHDG | 409 |
| Enterokinase | EDDDDKA | 410 |
| FAP | KQEQNPGST | 411 |
| Kallikrein 2 | GKAFRR | 412 |
| Plasmin | DAFK | 413 |
| Plasmin | DVLK | 414 |
| Plasmin | DAFK | 415 |
| TOP | ALLLALL | 416 |
| | GPLGVRG | 417 |
| | IPVSLRSG | 418 |
| | VPLSLYSG | 419 |
| | SGESPAYYTA | 420 |

Exemplary protease cleavable linkers include, but are not limited to kallikrein cleavable linkers, thrombin cleavable linkers, chymase cleavable linkers, carboxypeptidase A cleavable linkers, cathepsin cleavable linkers, elastase cleavable linkers, FAP cleavable linkers, ADAM cleavable linkers, PR-3 cleavable linkers, granzyme M cleavable linkers, a calpain cleavable linkers, a matrix metalloproteinase (MMP) cleavable linkers, a plasminogen activator cleavable linkers, a caspase cleavable linkers, a tryptase cleavable linkers, or a tumor cell surface protease. Specifically, MMP9 cleavable linkers, ADAM cleavable linkers, CTSL1 cleavable linkers, FAPα cleavable linkers, and cathepsin cleavable linkers. Some preferred protease-cleavable linkers are cleaved by a MMP and/or a cathepsin.

The linker sequences disclosed herein are typically less than 100 amino acids. Such linker sequences can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domains.

In some embodiments, the linker comprises the sequence

(SEQ ID NO: 195)
GPAGLYAQ;
(SEQ ID NO: 196)
GPAGMKGL;
(SEQ ID NO: 197)
PGGPAGIG;
(SEQ ID NO: 198)
ALFKSSFP;
(SEQ ID NO: 199)
ALFFSSPP;
(SEQ ID NO: 200)
LAQRLRSS;
(SEQ ID NO; 201)
LAQKLKSS;
(SEQ ID NO: 202)
GALFKSSFPSGGGPAGLYAQGGSGKGGSGK;
(SEQ ID NO: 203)
RGSGGGPAGLYAQGSGGGPAGLYAQGGSGK;
(SEQ ID NO: 204)
KGGGPAGLYAQGPAGLYAQGPAGLYAQGSR;
(SEQ ID NO: 205)
RGGPAGLYAQGGPAGLYAQGGGPAGLYAQK;
(SEQ ID NO: 206)
KGGALFKSSFPGGPAGIGPLAQKLKSSGGS;
(SEQ ID NO: 207)
SGGPGGPAGIGALFKSSFPLAQKLKSSGGG;
(SEQ ID NO: 208)
RGPLAQKLKSSALFKSSFPGGPAGIGGGGK;
(SEQ ID NO: 209)
GGGALFKSSFPLAQKLKSSPGGPAGIGGGR;
(SEQ ID NO: 210)
RGPGGPAGIGPLAQKLKSSALFKSSFPGGG;
(SEQ ID NO: 211)
RGGPLAQKLKSSPGGPAGIGALFKSSFPGK;
(SEQ ID NO: 212)
RSGGPAGLYAQALFKSSFPLAQKLKSSGGG;
(SEQ ID NO: 213)
GGPLAQKLKSSALFKSSFPGPAGLYAQGGR;
(SEQ ID NO: 214)
GGALFKSSFPGPAGLYAQPLAQKLKSSGGK;
(SEQ ID NO: 215)
RGGALFKSSFPLAQKLKSSGPAGLYAQGGK;
(SEQ ID NO: 216)
RGGGPAGLYAQPLAQKLKSSALFKSSFPGG;
SGPLAQKLKSSGPAGLYAQALFKSSFPGSK;
(SEQ ID NO: 218)
KGGPGGPAGIGPLAQRLRSSALFKSSFPGR;
(SEQ ID NO: 219)
KSGPGGPAGIGALFFSSPPLAQKLKSSGGR;
or
(SEQ ID NO: 220)
SGGFPRSGGSFNPRTFGSKRKRRGSRGGGG

Certain preferred linkers comprises the sequence GPAGLYAQ (SEQ ID NO: 195) or ALFKSSFP (SEQ ID NO: 198). The linkers disclosed herein can comprise one or more cleavage motif or functional variants that are the same or different. The linkers can comprise 1, 2, 3, 4, 5, or more cleavage motifs or functional variants. Linkers comprising 30 amino acids can contain 2 cleavage motifs or functional variants, 3 cleavage motifs or functional variants or more. A “functional variant” of a linker retains the ability to be cleaved with high efficiency at a target site (e.g., a tumor microenvironment that expresses high levels of the protease) and are not cleaved or cleaved with low efficiency in the periphery (e.g., serum). For example, the functional variants retain at least about 50%, about 55%, about 60%, about 70%, about 80%, about 85%, about 95% or more of the cleavage efficiency of a linker comprising any one of SEQ ID NOs: 195-220 or 447-448.

The linkers comprising more than one cleavage motif can be selected from SEQ ID NOs: 195-201 or 447-448 and combinations thereof. Preferred linkers comprising more than one cleavage motif comprise the amino acids selected from SEQ ID NO: 202-220.

The linker can comprise both ALFKSSFP (SEQ ID NO: 198) and GPAGLYAQ (SEQ ID NO: 195). The linker can comprise two cleavage motifs that each have the sequence GPAGLYAQ (SEQ ID NO: 195). Alternatively or additionally, the linker can comprise two cleavage motifs that each have the sequence ALFKSSFP (SEQ ID NO: 198). The linker can comprise a third cleavage motif that is the same or different.

In some embodiments, the linker comprises an amino acid sequence that is at least about 90%, at least about 95%, at least about 96%, at least about 97%, at least about 98%, or at least 99% identical to SEQ ID NOs: 195 to SEQ ID NO: 220 or 447-448 over the full length of SEQ ID NO: 195-220 or SEQ ID NOS 447-448.

The disclosure also relates to functional variants of the linkers comprising SEQ ID NOs: 195-220 or 447-448. The functional variants of the linkers comprising SEQ ID NOs: 195-220 or 447-448 generally differ from SEQ ID NOs: 195-220 or 447-448 by one or a few amino acids (including substitutions, deletions, insertions, or any combination thereof), and substantially retain their ability to be cleaved by a protease.

The functional variants can contain at least one or more amino acid substitutions, deletions, or insertions relative to the linkers comprising SEQ ID NOs: 195-220 or 447-448. The functional variant can comprise 1, 2, 3, 4, 5, 6, 7, 8, 9, or 10 amino acid alterations comparted to the linkers comprising SEQ ID NOs: 195-220 or 447-448. In some preferred embodiments, the functional variant differs from the linker comprising SEQ ID NOs: 195-220 by less than 10, less, than 8, less than 5, less than 4, less than 3, less than 2, or one amino acid alterations, e.g., amino acid substitutions or deletions. In other embodiments, the functional variant may comprise 1, 2, 3, 4, 5, 6, 7, 8, 9, or 10 amino acid substitutions compared to SEQ ID NOs: 195-220 or 447-448. The amino acid substitution can be a conservative substitution or a non-conservative substitution, but preferably is a conservative substitution.

In other embodiments, the functional variants of the linkers may comprise 1, 2, 3, 4, or 5 or more non-conservative amino acid substitutions compared to the linkers comprising SEQ ID NOs: 195-220 or 447-448. Non-conservative amino acid substitutions could be recognized by one of skill in the art. The functional variant of the linker preferably contains no more than 1, 2, 3, 4, or 5 amino acid deletions.

The amino acid sequences disclosed in the linkers can be described by the relative linear position in the linker with respect to the sissile bond. As will be well-understood by persons skilled in the art, linkers comprising 8 amino acid protease substrates (e.g., SEQ ID Nos: 195-201 or 447-448) contain amino acid at positions P4, P3, P2, P1, P1′, P2′, P3′, P4′, wherein the sissile bond is between P1 and P1′. For example, amino acid positions for the linker comprising the sequence GPAGLYAQ (SEQ ID NO: 195) can be described as follows:

| G | P | A | G | L | Y | A | Q |
|---|---|---|---|---|---|---|---|
| P4 | P3 | P2 | P1 | P1′ | P2′ | P3′ | P4′ |

“GPAGLYAQ” disclosed as SEQ ID NO: 195.

Amino acids positions for the linker comprising the sequence ALFKSSFP (SEQ ID NO: 198) can be described as follows:

| A | L | F | K | S | S | F | P |
|---|---|---|---|---|---|---|---|
| P4 | P3 | P2 | P1 | P1′ | P2′ | P3′ | P4′ |

“ALFKSSFP” disclosed as SEQ ID NO: 198.

Preferably, the amino acids surrounding the cleavage site (e.g., positions P1 and P1′ for SEQ ID NOs: 195-201 or 447-448) are not substituted.

In embodiments, the linker comprises the sequence GPAGLYAQ (SEQ ID NO: 195) or ALFKSSFP (SEQ ID NO: 198) or a functional variant of SEQ ID NO: 195 or a function variant of SEQ ID NO: 198. As described herein, a functional variant of PAGLYAQ (SEQ ID NO: 447) or ALFKSSFP (SEQ ID NO: 198) can comprise one or more amino acid substitutions, and substantially retain their ability to be cleaved by a protease. Specifically, the functional variants of GPAGLYAQ (SEQ ID NO: 195) is cleaved by MMP14, and the functional variant of ALFKSSFP (SEQ ID NO: 198) is cleaved by Capthepsin L (CTSL1). The functional variants also retain their ability to be cleaved with high efficiency at a target site (e.g., a tumor microenvironment that expresses high levels of the protease). For example, the functional variants of GPAGLYAQ (SEQ ID NO: 195) or ALFKSSFP (SEQ ID NO: 198) retain at least about 50%, about 55%, about 60%, about 70%, about 80%, about 85%, about 95% or more of the cleavage efficiency of a linker comprising amino acid sequence GPAGLYAQ (SEQ ID NO: 195) or ALFKSSFP (SEQ ID NO: 198), respectively.

Preferably, the functional variant of GPAGLYAQ (SEQ ID NO: 195) or ALFKSSFP (SEQ ID NO: 198) comprise no more than 1, 2, 3, 4, or 5 conservative amino acid substitutions compared to GPAGLYAQ (SEQ ID NO: 195) or ALFKSSFP (SEQ ID NO: 198). Preferably, the amino acids at position P1 and P1′ are not substituted. The amino acids at positions P1 and P1′ in SEQ ID NO: 195 are G and L, and the amino acids at positions P1 and P1′ in SEQ ID NO: 198 are K and S.

The functional variant of GPAGLYAQ (SEQ ID NO: 195) can preferably comprise one or more of the following: a) an arginine amino acid substitution at position P4, b) a leucine, valine, asparagine, or proline amino acid substitution at position P3, c) a asparagine amino acid substitution at position P2, d) a histidine, asparagine, or glycine amino acid substitution at position P1, e) a asparagine, isoleucine, or leucine amino acid substitution at position P1′, f) a tyrosine or arginine amino acid substitution at position P2′, g) a glycine, arginine, or alanine amino acid substitution at position P3′, h) or a serine, glutamine, or lysine amino acid substitution at position P4′. The following amino acid substitutions are disfavored in functional variants of GPAGLYAQ (SEQ ID NO: 195): a) arginine or isoleucine at position P3, b) alanine at position P2, c) valine at position P1, d) arginine, glycine, asparagine, or threonine at position P1′, e) aspartic acid or glutamic acid at position P2′, f) isoleucine at position P3′, g) valine at position P4′. In some embodiments, the functional variant of GPAGLYAQ (SEQ ID NO: 195) does not comprise an amino acid substitution at position P1 and/or P1′.

The amino acid substitution of the functional variant of GPAGLYAQ (SEQ ID NO: 195) preferably comprises an amino acid substitution at position P4 and/or P4′. For example, the functional variant of GPAGLYAQ (SEQ ID NO: 195) can comprise a leucine at position P4, or serine, glutamine, lysine, or phenylalanine at position P4. Alternatively or additionally, the functional variant of GPAGLYAQ (SEQ ID NO: 195) can comprise a glycine, phenylalanine, or a proline at position P4′.

In some embodiments, the amino acid substitutions at position P2 or P2′ of GPAGLYAQ (SEQ ID NO: 195) are not preferred.

In some embodiments, the functional variant of GPAGLYAQ (SEQ ID NO: 195) comprises the amino acid sequence selected from SEQ ID NOs: 221-295. Specific functional variants of GPAGLYAQ (SEQ ID NO: 195) include GPLGLYAQ (SEQ ID NO: 259), and GPAGLKGA (SEQ ID NO: 249).

The functional variants of LFKSSFP (SEQ ID NO: 448) preferably comprises hydrophobic amino acid substitutions. The functional variant of LFKSSFP (SEQ ID NO: 448) can preferably comprise one or more of the following: (a) lysine, histidine, serine, glutamine, leucine, proline, or phenylalanine at position P4; (b) lysine, histidine, glycine, proline, asparagine, phenylalanine at position P3; (c) arginine, leucine, alanine, glutamine, or histatine at position P2; (d) phenylalanine, histidine, threonine, alanine, or glutamine at position P1; (e) histidine, leucine, lysine, alanine, isoleucine, arginine, phenylalanine, asparagine, glutamic acid, or glycine at position P1′, (f) phenylalanine, leucine, isoleucine, lysine, alanine, glutamine, or proline at position P2′; (g) phenylalanine, leucine, glycine, serine, valine, histidine, alanine, or asparagine at position P3′; and phenylalanine, histidine, glycine, alanine, serine, valine, glutamine, lysine, or leucine.

The inclusion of aspartic acid and/or glutamic acid in functional variants of SEQ ID NO: 448 are generally disfavored and avoided. The following amino acid substitutions are also disfavored in functional variants of LFKSSFP (SEQ ID NO: 448): (a) alanine, serine, or glutamic acid at position P3; (b) proline, threonine, glycine, or aspartic acid at position P2; (c) proline at position P1; (d) proline at position P1′; (e) glycine at position P2′; (f) lysine or glutamic acid at position P3′; (g) aspartic acid at position P4′.

The amino acid substitution of the functional variant of LFKSSFP (SEQ ID NO: 448) preferably comprises an amino acid substitution at position P4 and/or P1. In some embodiments, an amino acid substitution of the functional variant of LFKSSFP (SEQ ID NO: 448) at position P4′ is not preferred.

In some embodiments, the functional variant of LFKSSFP (SEQ ID NO: 448) comprises the amino acid sequence selected from SEQ ID NOs: 296-374. Specific functional variants of LFKSSFP (SEQ ID NO: 448) include ALFFSSPP (SEQ ID NO: 199), ALFKSFPP (SEQ ID NO: 346), ALFKSLPP (SEQ ID NO: 347); ALFKHSPP (SEQ ID NO: 335); ALFKSIPP (SEQ ID NO: 348); ALFKSSLP (SEQ ID NO: 356); or SPFRSSRQ (SEQ ID NO: 297).

The linkers disclosed herein can form a stable prodrug under physiological conditions with the amino acid sequences (e.g. domains) that they link, while being capable of being cleaved by a protease. For example, the linker is stable (e.g., not cleaved or cleaved with low efficiency) in the circulation and cleaved with higher efficiency at a target site (i.e. a tumor microenvironment). Accordingly, fusion polypeptides that include the linkers disclosed herein can, if desired, have a prolonged circulation half-life and/or lower biological activity in the circulation in comparison to the components of the fusion polypeptide as separate molecular entities. Yet, when in the desired location (e.g., tumor microenvironment) the linkers can be efficiently cleaved to release the components that are joined together by the linker and restoring or nearly restoring the half-life and biological activity of the components as separate molecular entities.

The linker desirably remains stable in the circulation for at least 2 hours, at least 5, hours, at least 10 hours, at least 15 hours, at least 20 hours, at least 24 hours, at least 30 hours, at least 35 hours, at least 40 hours, at least 45 hours, at least 50 hours, at least 60 hours, at least 65 hours, at least 70 hours, at least 80 hours, at least 90 hours, or longer.

In some embodiments, the linker is cleaved by less than 90%, 80%, 70%, 60%, 50%, 40%, 30%, 20%, 20%, 5%, or 1% in the circulation as compared to the target location. The linker is also stable in the absence of an enzyme capable of cleaving the linker. However, upon expose to a suitable enzyme (i.e., a protease), the linker is cleaved resulting in separation of the linked domain.

### E. Pharmaceutical Compositions

Also provided herein, are pharmaceutical compositions comprising a IFN polypeptide prodrug described herein, a vector comprising the polynucleotide encoding the IFN polypeptide prodrug or a host cell transformed by this vector and at least one pharmaceutically acceptable carrier.

Provided herein are pharmaceutical formulations or compositions containing the IFN polypeptide prodrugs as described herein and a pharmaceutically acceptable carrier. Compositions comprising the IFN polypeptide prodrugs as described herein are suitable for administration in vitro or in vivo. The term “pharmaceutically acceptable carrier” includes, but is not limited to, any carrier that does not interfere with the effectiveness of the biological activity of the ingredients and that is not toxic to the subject to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Suitable carriers and their formulations are described in Remington: The Science and Practice of Pharmacy, 21st Edition, David B. Troy, ed., Lippicott Williams & Wilkins (2005). Typically, an appropriate amount of a pharmaceutically-acceptable salt is used in the formulation to render the formulation isotonic, although the formulate can be hypertonic or hypotonic if desired. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the IFN or polypeptide prodrugs or nucleic acid sequences encoding the IFN polypeptide prodrugs to humans or other subjects.

In some embodiments of the pharmaceutical compositions, the inducible IFN prodrug described herein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the inducible IFN prodrug is attached to liposomes. In some instances, the inducible IFN prodrugs are conjugated to the surface of liposomes. In some instances, the inducible IFN prodrug are encapsulated within the shell of a liposome. In some instances, the liposome is a cationic liposome.

The IFN polypeptide prodrugs described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

Optionally, the inducible IFN prodrug or nucleic acid sequences encoding the inducible IFN prodrug are administered by a vector. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein. Such compositions and methods can be used to transfect or transduce cells in vitro or in vivo, for example, to produce cell lines that express and preferably secrete the encoded chimeric polypeptide or to therapeutically deliver nucleic acids to a subject. The components of the IFN polypeptide disclosed herein are typically operably linked in frame to encode a fusion protein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general and methods of making them are described by Coffin et al., Retroviruses, Cold Spring Harbor Laboratory Press (1997). The construction of replication-defective adenoviruses has been described (Berkner et al., J. Virol. 61:1213-20 (1987); Massie et al., Mol. Cell. Biol. 6:2872-83 (1986); Haj-Ahmad et al., J. Virol. 57:267-74 (1986); Davidson et al., J. Virol. 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided IFN polypeptide prodrugs and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The IFN polypeptide prodrugs disclosed herein can be delivered by subviral dense bodies (DBs). DBs transport proteins into target cells by membrane fusion. Methods for making and using DBs are described in, for example, Pepperl-Klindworth et al., Gene Therapy 10:278-84 (2003). The provided polypeptides can be delivered by tegument aggregates. Methods for making and using tegument aggregates are described in International Publication No. WO 2006/110728.

Non-viral based delivery methods, can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Pal Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Such vectors can also be used to make the IFN polypeptide prodrugs by expression in a suitable host cell, such as CHO cells.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g., β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

### F. Therapeutic Applications

Also provided herein, are methods and uses for the treatment of a disease, disorder or condition associated with a target antigen comprising administering to a subject in need thereof a inducible IFN prodrug as described herein. Diseases, disorders, or conditions include, but are not limited to, cancer, inflammatory disease, an immunological disorder, autoimmune disease, infectious disease (i.e., bacterial, viral, or parasitic disease). Preferably, the disease, disorder, or condition is cancer.

Any suitable cancer may be treated with the IFN polypeptide prodrugs provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. In embodiments, the cancer is melanoma or breast cancer.

In some embodiments, provided herein is a method of enhancing an immune response in a subject in need thereof by administering an effective amount of an inducible IFN prodrug provided herein to the subject. The enhanced immune response may prevent, delay, or treat the onset of cancer, a tumor, or a viral disease. Without being bound by theory, the inducible IFN prodrug enhances the immune response by activating the innate and adaptive immunities. In some embodiments, the methods described herein increase the activity of Natural Killer Cells and T lymphocytes. In some embodiments, the inducible IFN prodrug provided herein, can induce IFNγ release from Natural Killer cells as well as CD4+ and CD8+ T cells.

The method can further involve the administration of one or more additional agents to treat cancer, such as chemotherapeutic agents (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, Procarabizine), immuno-oncology agents (e.g., anti-PD-L1, anti-CTLA4, anti-PD-1, anti-CD47, anti-GD2), cellular therapies (e.g., CAR-T, T-cell therapy), oncolytic viruses and the like. Non-limiting examples of anti-cancer agents that can be used include acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1 interferon alpha-n3; interferon beta-I; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinzolidine sulfate; vinzolidine sulfate; vorozole; zeniplatin; zinostatin; zorubicin hydrochloride.

In some embodiments of the methods described herein, the inducible IFN prodrug or the inducible IFN prodrug is administered in combination with an agent for the treatment of the particular disease, disorder, or condition. Agents include, but are not limited to, therapies involving antibodies, small molecules (e.g., chemotherapeutics), hormones (steroidal, peptide, and the like), radiotherapies (γ-rays, C-rays, and/or the directed delivery of radioisotopes, microwaves, UV radiation and the like), gene therapies (e.g., antisense, retroviral therapy and the like) and other immunotherapies. In some embodiments, the inducible IFN prodrug or is administered in combination with anti-diarrheal agents, anti-emetic agents, analgesics and/or non-steroidal anti-inflammatory agents.

### G. Definitions

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

As used herein, the terms “activatable,” “activate,” “induce,” and “inducible” refers to a inducible IFN prodrug that has an attenuated activity form (e.g., attenuated receptor binding and/or agonist activity) and an activated form. The inducible IFN prodrug is activated by protease cleavage of the linker that causes the blocking element and half-life extension element to dissociate from the inducible IFN prodrug. The induced/activated IFN prodrug can bind with increased affinity/avidity to the IFN receptor.

The terms “antibody” and “immunoglobulin” are used interchangeably herein. An antibody or immunoglobulin, as used herein, is intended to refer to immunoglobulin molecules comprised of two heavy (H) chains. Typically, antibodies in mammals (e.g., humans, rodents, and monkey's) comprise four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. Antibodies can include, for example, monoclonal antibodies, recombinantly produced antibodies, monospecific antibodies, multi specific antibodies (including bispecific antibodies), human antibodies, humanized antibodies, chimeric antibodies, immunoglobulins, synthetic antibodies, or tetrameric antibodies comprising two heavy chain and two light chain molecules. One of skill in the art would recognize that other forms of antibodies exist (e.g. camelid and shark antibodies).

The term “attenuated” as used herein is an IFN receptor agonist that has decreased receptor agonist activity as compared to the IFN receptor's naturally occurring agonist. An attenuated IFN agonist can have at least about 10×, at least about 50×, at least about 100×, at least about 250×, at least about 500×, at least about 1000× or less agonist activity as compared to the receptor's naturally occurring agonist. When a inducible IFN prodrug that contains IFN as described herein is described as “attenuated” or having “attenuated activity”, it is meant that the inducible IFN prodrug is an attenuated IFN receptor agonist.

The term “cancer” refers to the physiological condition in mammals in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer), osteosarcoma, melanoma, colon cancer, colorectal cancer, endometrial (e.g., serous) or uterine cancer, salivary gland carcinoma, kidney cancer, liver cancer, prostate cancer, vulval cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu.

A “conservative” amino acid substitution, as used herein, generally refers to substitution of one amino acid residue with another amino acid residue from within a recognized group which can change the structure of the peptide but biological activity of the peptide is substantially retained. Conservative substitutions of amino acids are known to those skilled in the art. Conservative substitutions of amino acids can include, but not limited to, substitutions made amongst amino acids within the following groups: (a) M, I, L, V; (b) F, Y, W; (c) K, R, H; (d) A, G; (e) S, T; (f) Q, N; and (g) E, D. For instance, a person of ordinary skill in the art reasonably expect that an isolated replacement of a leucine with an isoleucine or valine, an aspartate with a glutamate, a threonine with a serine, or a similar replacement of an amino acid with a structurally related amino acid will not have a major effect on the biological activity of the resulting molecule.

As used herein, the term “half-life extension element” in the context of the inducible IFN prodrug disclosed herein, refers to a chemical element, preferable a polypeptide that increases the serum half-life and improve pK, for example, by altering its size (e.g., to be above the kidney filtration cutoff), shape, hydrodynamic radius, charge, or parameters of absorption, biodistribution, metabolism, and elimination.

As used herein, the term “operably linked” in the context of a inducible IFN prodrug refers to the orientation of the components of a inducible IFN prodrug that permits the components to function in their intended manner. For example, a polypeptide comprising an IFN subunit and an IFN blocking element are operably linked by a protease cleavable linker in a inducible IFN prodrug when the IFN blocking element is capable of inhibiting the IFN receptor-activating activity of the IFN polypeptide, but upon cleavage of the protease cleavable linker the inhibition of the IFN receptor-activating activity of the IFN polypeptide by the IFN blocking element is decreased or eliminated, for example because the IFN blocking element can diffuse away from the IFN.

As used herein, the terms “peptide”, “polypeptide”, or “protein” are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

The term “subject” herein to refers to any animal, such as any mammal, including but not limited to, humans, non-human primates, rodents, and the like. In some embodiments, the mammal is a mouse. In some embodiments, the mammal is a human.

As used herein, the term “therapeutically effective amount” refers to an amount of a compound described herein (i.e., a inducible IFN prodrug) that is sufficient to achieve a desired pharmacological or physiological effect under the conditions of administration. For example, a “therapeutically effective amount” can be an amount that is sufficient to reduce the signs or symptoms of a disease or condition (e.g., a tumor). Those skilled in the art will appreciate that the therapeutic effects need not be complete or curative, as long as some benefit is provided to the subject. A therapeutically effective amount of a pharmaceutical composition can vary according to factors such as the disease state, age, sex, and weight of the individual, and the ability of the pharmaceutical composition to elicit a desired response in the individual. An ordinarily skilled clinician can determine appropriate amounts to administer to achieve the desired therapeutic benefit based on these and other considerations.

### 6. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### 7. EXAMPLES

The present invention is further described by the following examples, which are not intended to be limiting in any way.

### Example 1: HEK-Blue Assay

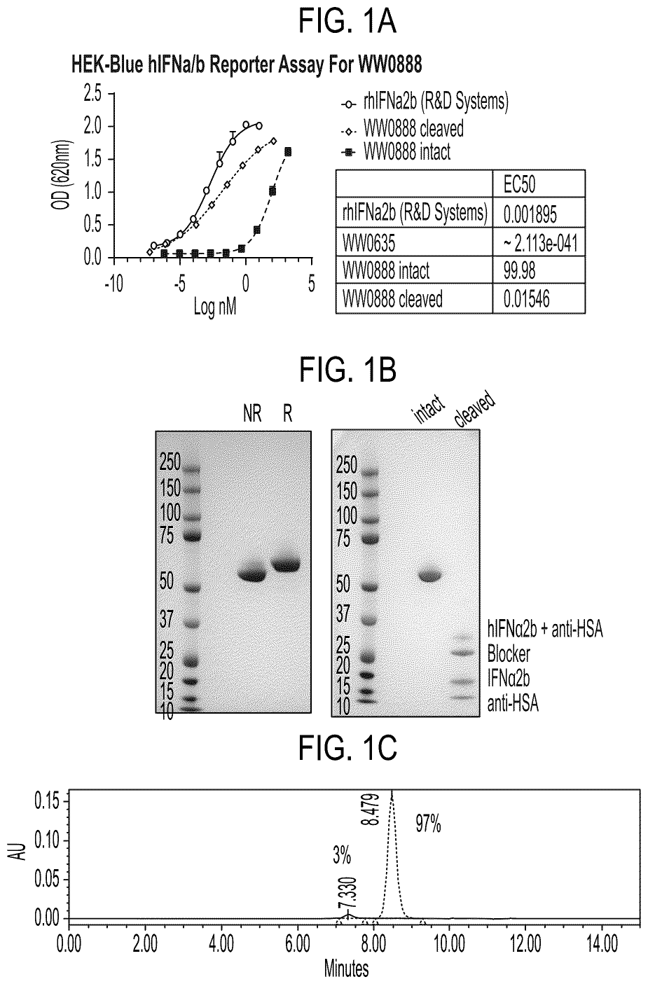

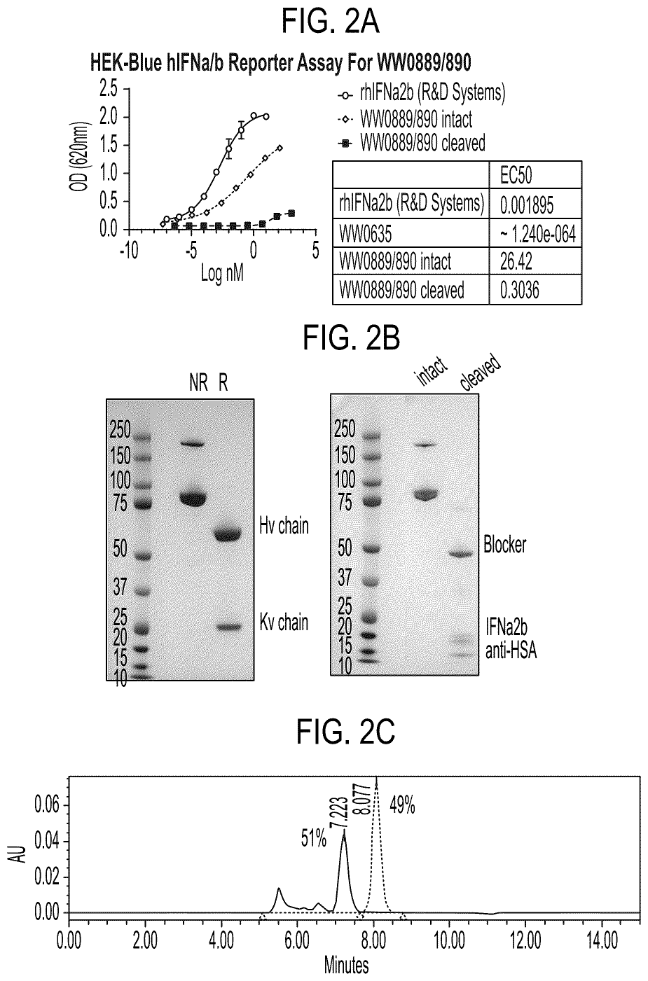

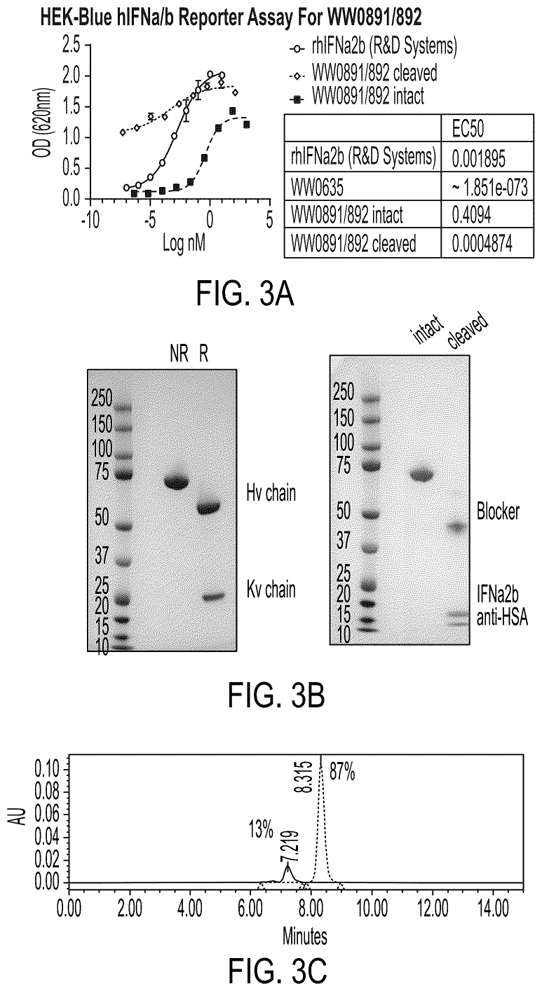

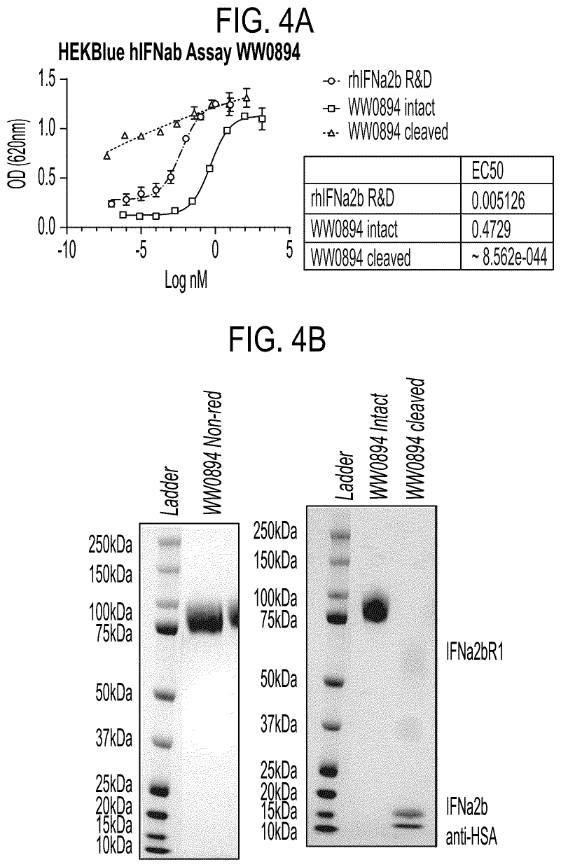

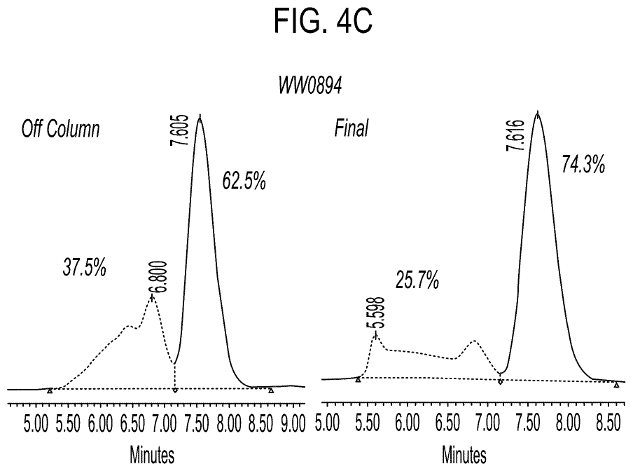

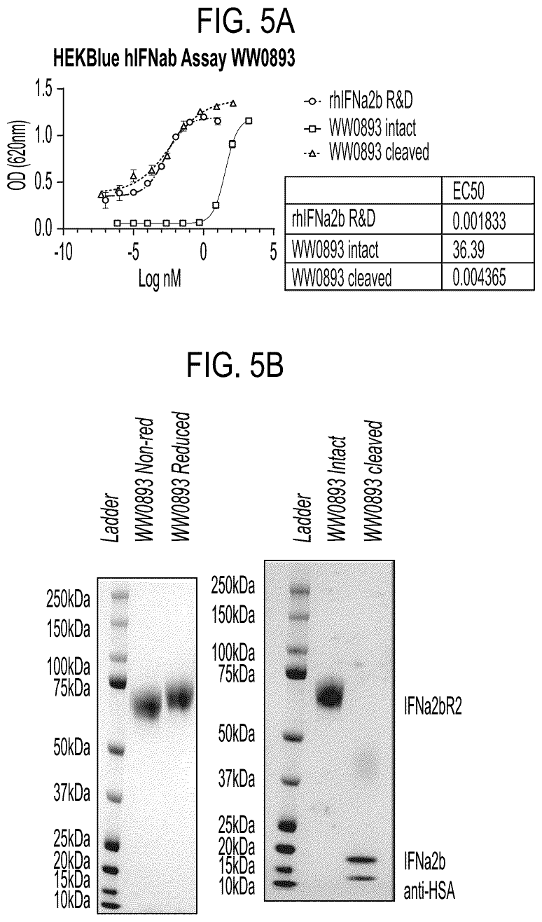

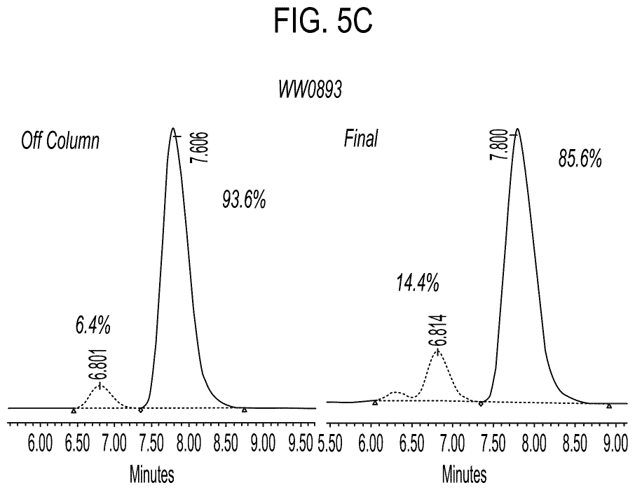

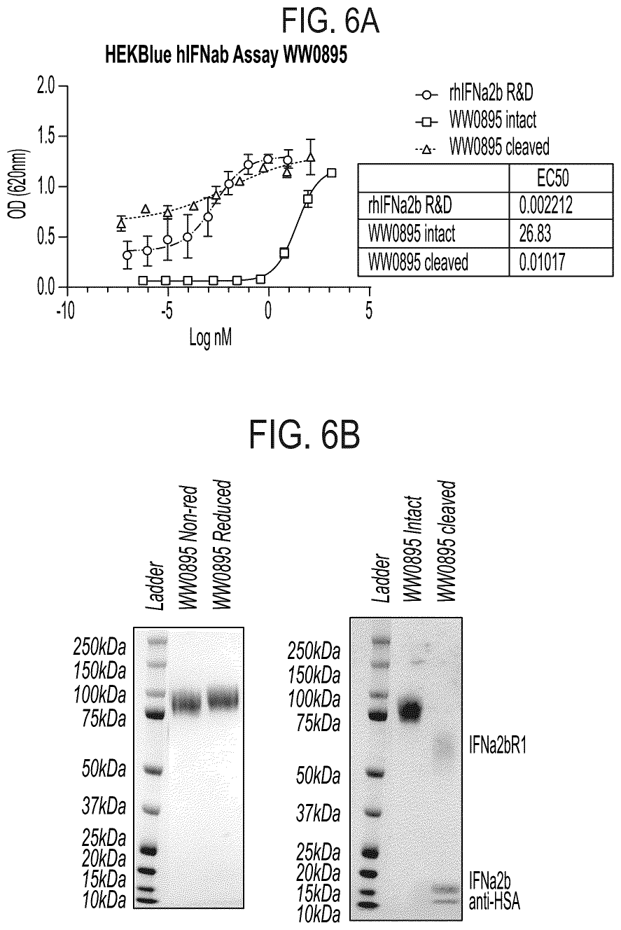

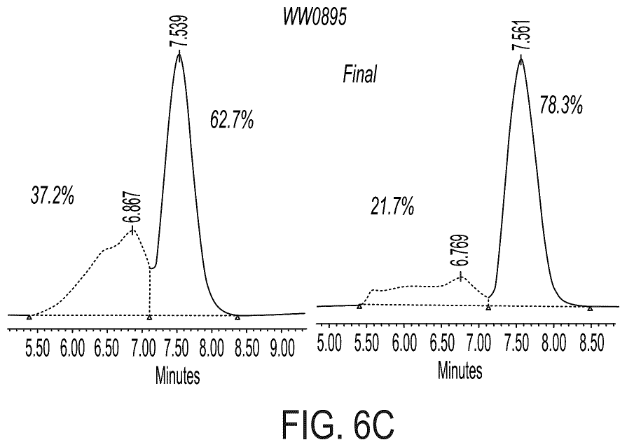

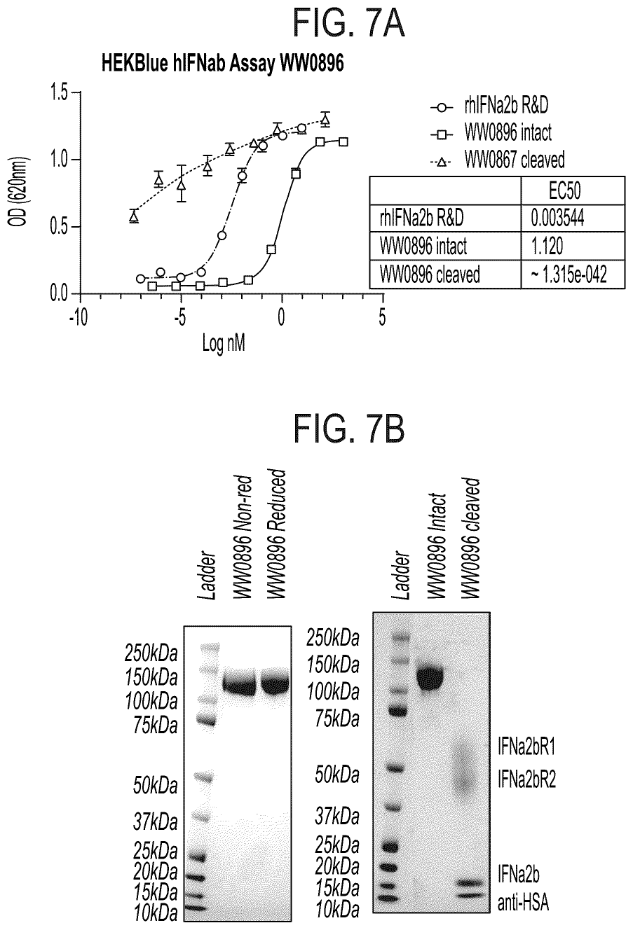

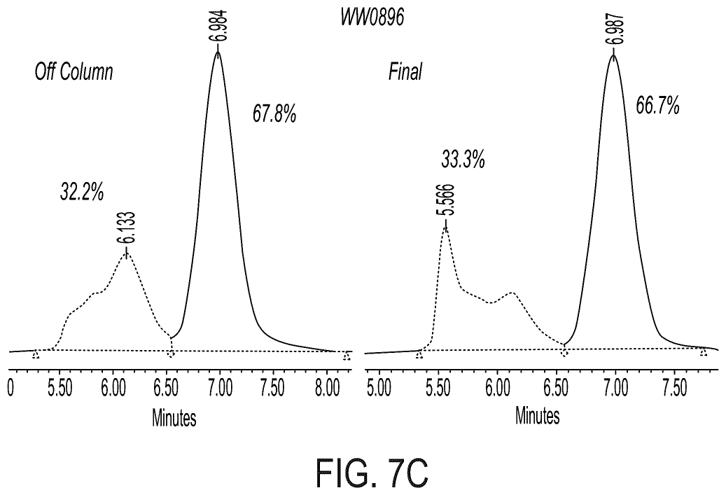

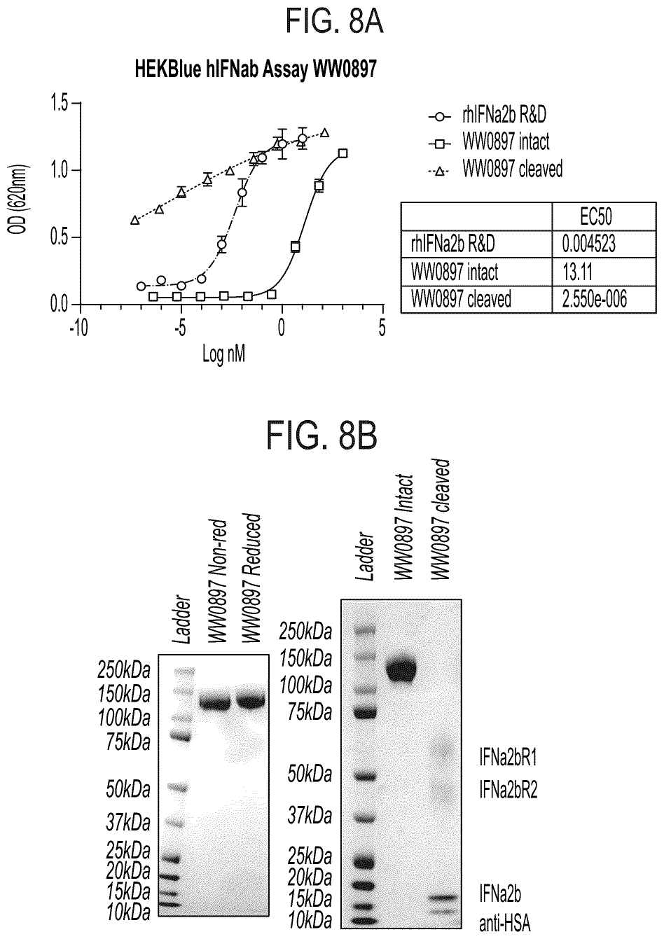

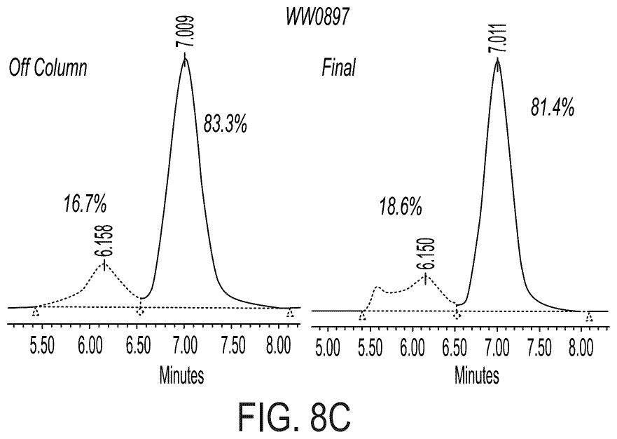

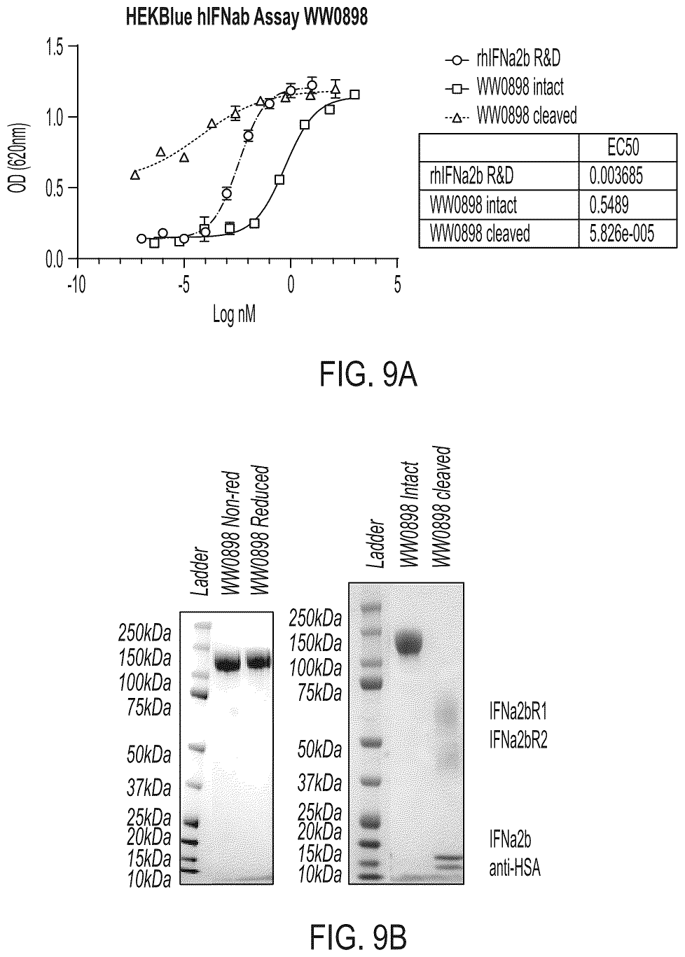

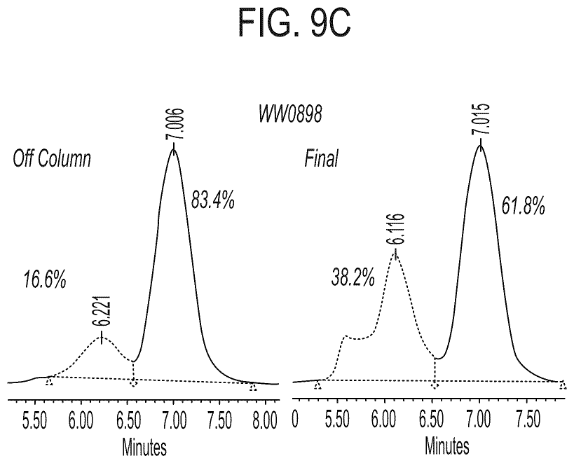

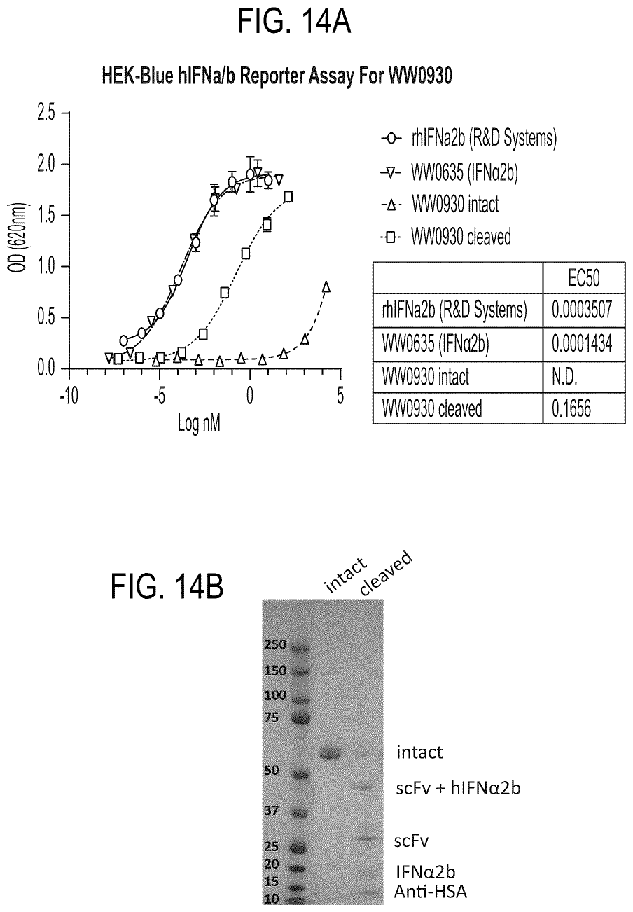

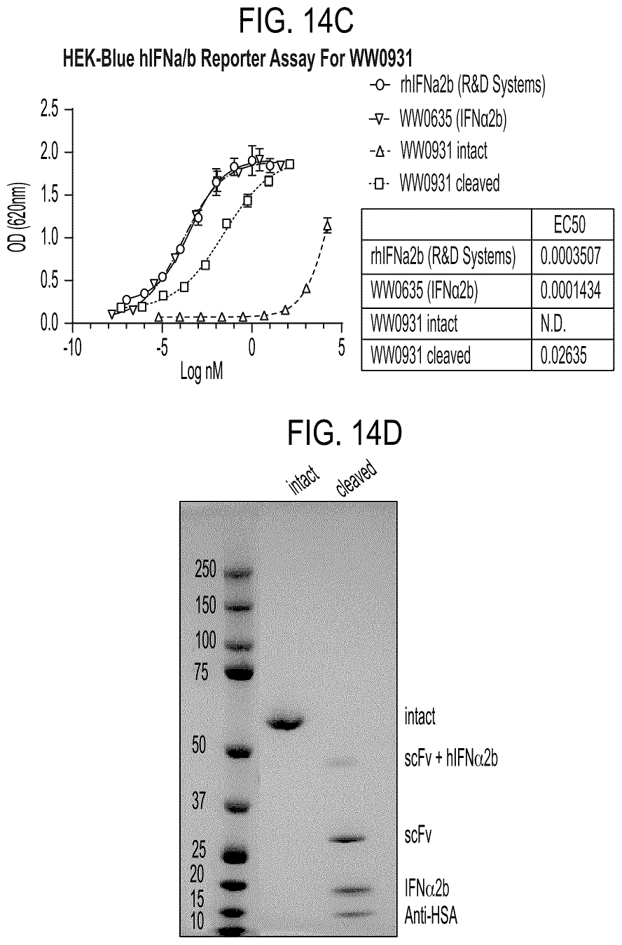

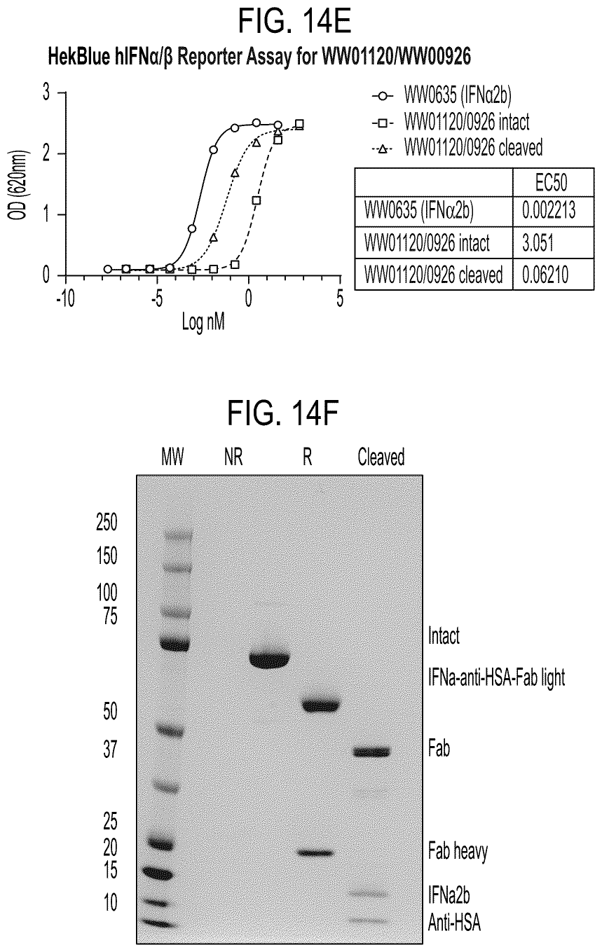

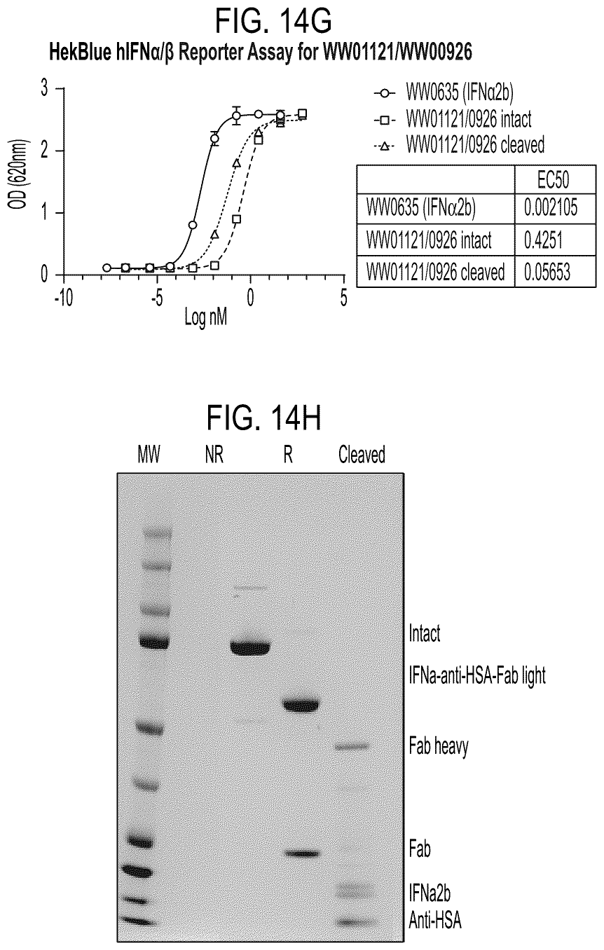

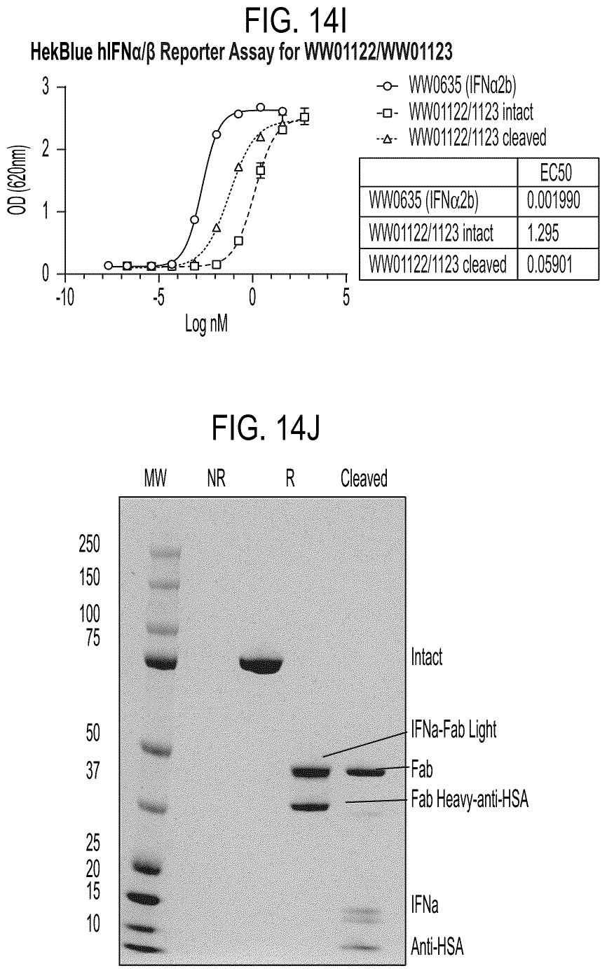

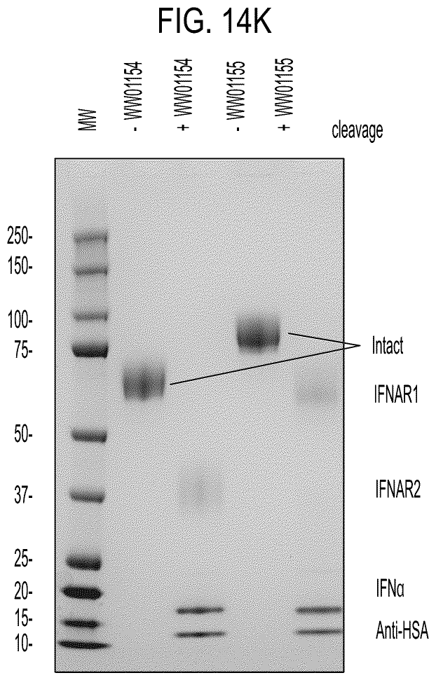

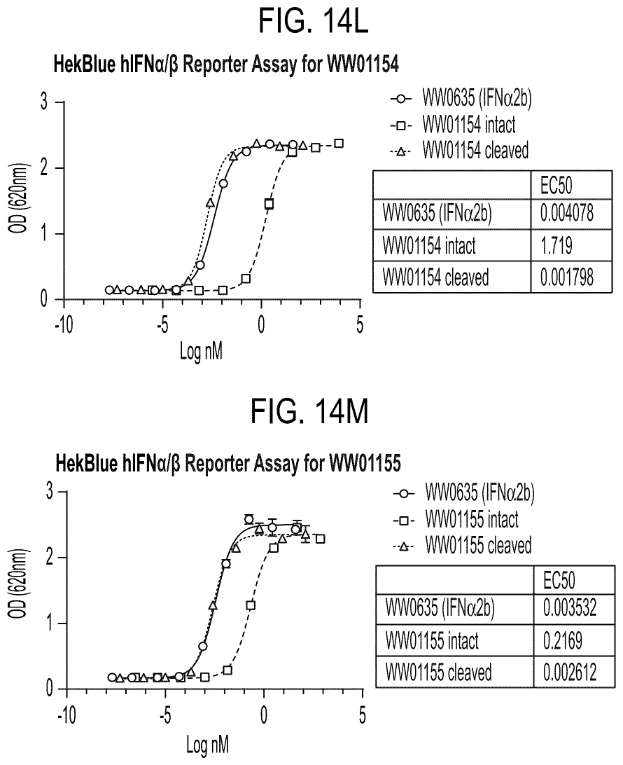

HEK-Blue IFN-α/β cells (InvivoGen) were plated in suspension at a density of 50,000 cells/well in culture media with or without 15 mg/ml human serum albumin (HSA) and stimulated with a dilution series IFN α and activatable human IFN α for 18 hours at 37° C. and 5% CO2. Activity of uncleaved and cleaved activatable IFNα was tested. Cleaved inducible IFNα was generated by incubation with active recombinant protease. Stimulation of HEK-Blue IFN-α/β cells with IFN α induces expression of Secreted Alkaline Phosphatase (SEAP) from an ISG54-SEAP reporter. IFNα activity was assessed by quantification of SEAP activity using the reagent QUANTI-Blue (InvivoGen), a colorimetric based assay. Results are shown in FIGS. 1A, 2A, 3A, 4A, 5A, 6A, 7A, 8A, 9A, 14A, 14C, 14E, 14G, 14I, 14L, and 14M.

### Example 2: Protease Cleavage of Fusion Protein by CTSL or Elastase Protease

One of skill in the art would be familiar with methods of setting up protein cleavage assay. 50 μg of protein in 1×PBS pH 7.4 were cleaved with 1 μg active CTSL (R&D Systems catalog #952-CY-010) or μg active elastase (Sigma catalog #324682) in a total volume of 100 μL and incubated at room temperature for up to 16 hours. Digested protein was subsequently used in functional assays or stored at −80° C. prior to testing. Extent of cleavage was monitored by SDS PAGE using methods well known in the art. As shown in FIGS. 1B, 2B, 3B, 4B, 5B, 6B, 7B, 8B, 9B, 13B, 13D, 13E, 14B, 14D, 14F, 14H, 14J, and 14K, full cleavage of the fusion proteins by CTSL or elastase protease was seen.

### Example 3: 16-Blue IFN-α/β Reporter Assay

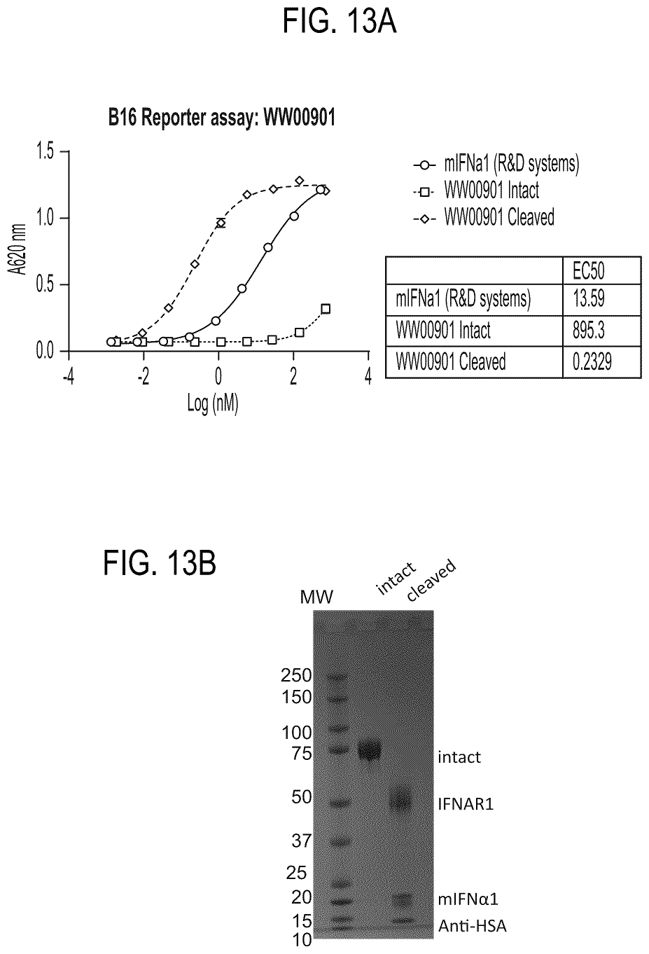

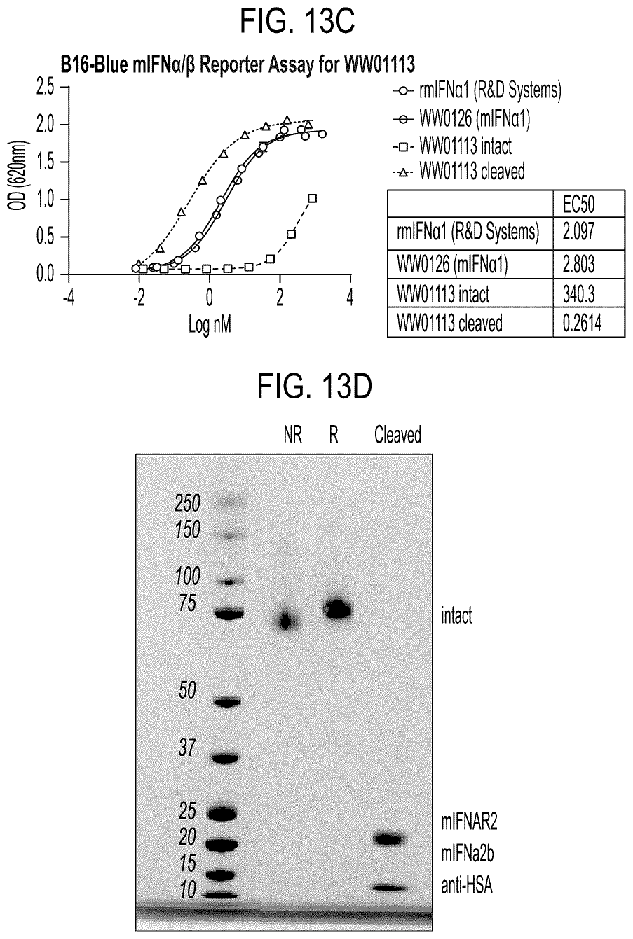

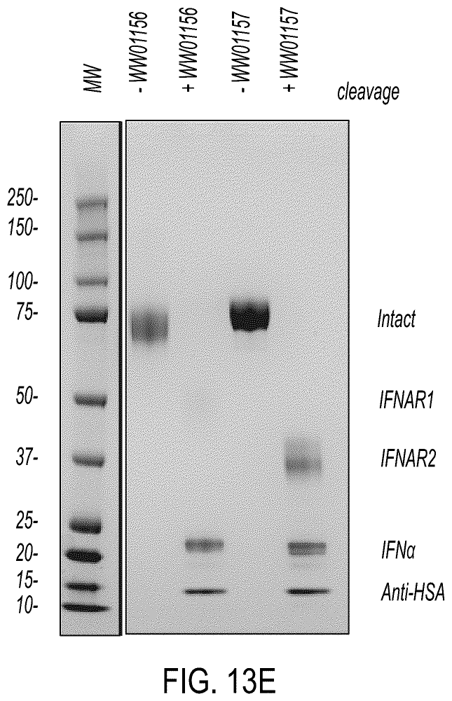

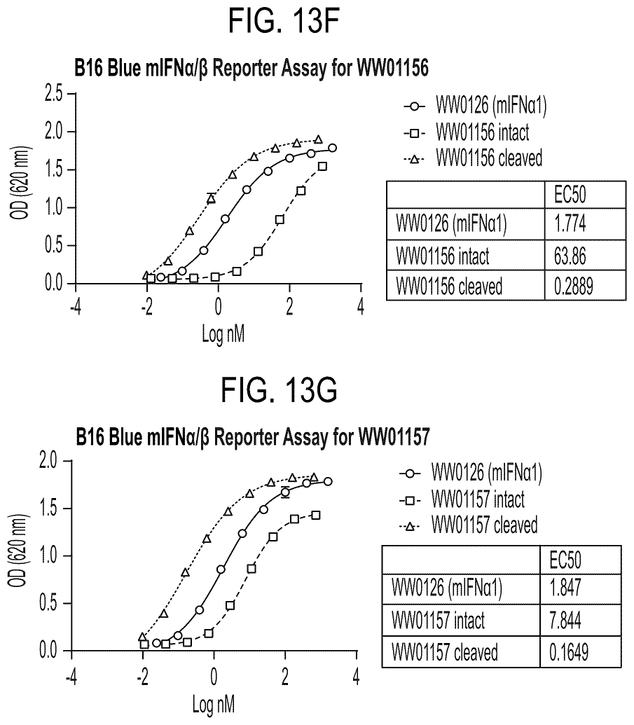

B16-Blue IFN-α/β cells (InvivoGen) will be plated in suspension at a density of 75,000 cells/well in culture media with or without 15 mg/ml mouse serum albumin (HSA) and stimulated with a dilution series of recombinant mouse IFNα and activatable mouse IFNα for 20-24 hours at 37° C. and 5% CO2. Activity of uncleaved and cleaved activatable IFNα will be tested. Cleaved inducible IFNα will be generated by incubation with active recombinant protease. Stimulation of B16-Blue IFN-α/β cells with IFNα will induce expression of Secreted Alkaline Phosphatase (SEAP) from an ISRE-ISG54-SEAP reporter. IFNα activity will be assessed by quantification of SEAP activity using the reagent QUANTI-Blue (InvivoGen), a colorimetric based assay. Results are shown in FIGS. 13A-13G.

### Example 4: Sec Analysis

Proteins were analyzed by analytical SEC for high molecular weight species quantitation to characterize purity. A Waters XBridge BEH sizing column was used for SEC. In short, 20 μg of protein was injected on the column and eluted under isocratic conditions in 100 mM sodium phosphate pH7 for 15 minutes.

### Example 5: MC38 Experiments (Study MC38-e655)

The MC38 cell line, a rapidly growing colon adenocarcinoma cell line, was used as a tumor model to examine the ability of fusion proteins to affect tumor growth and body weight.

TABLE 3
Agents and Treatment.

| Group | N | Agent | Dose | Route | Schedule |
|---|---|---|---|---|---|
| 1 | 8 | Vehicle | — | ip | biwk × 2 |
| 2 | 8 | WW00901 | 75 ug/animal | ip | biwk × 2 |
| 3 | 8 | WW00901 | 300 ug/animal | ip | biwk × 2 |
| 4 | 8 | WW00901 | 600 ug/animal | ip | biwk × 2 |

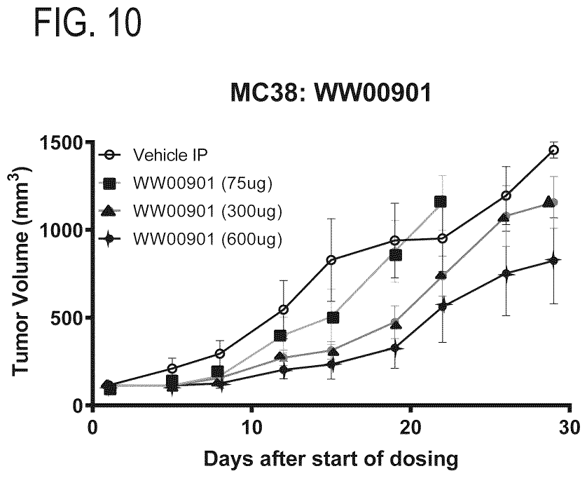

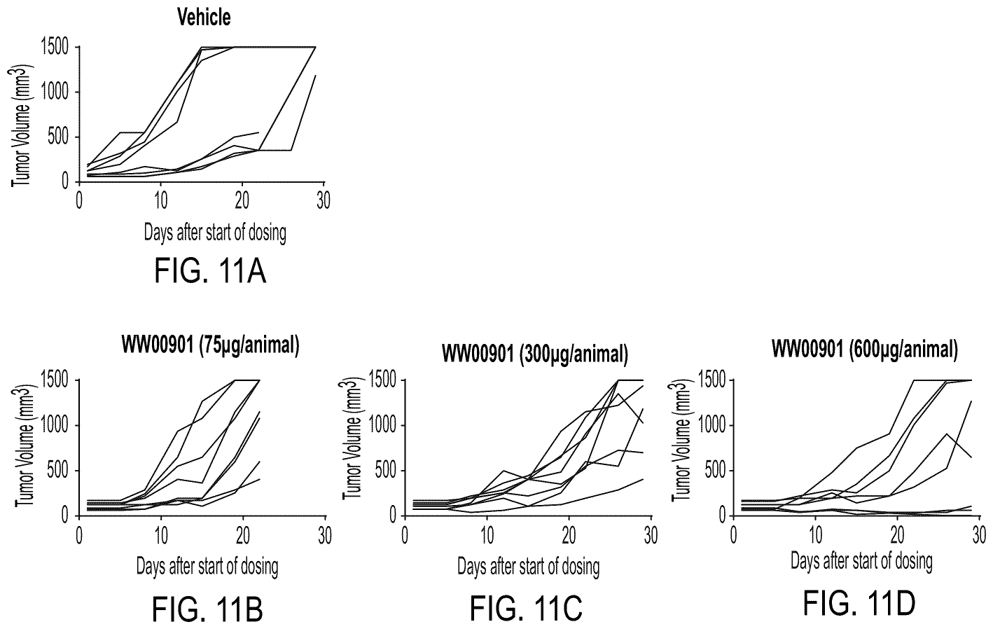

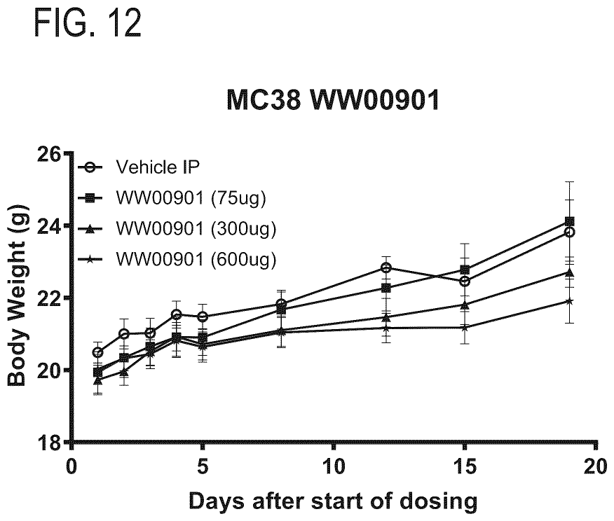

Mice were anaesthetized with isoflurane for implant of cells to reduce the ulcerations. Female C57BL/6 mice were set up with 5×10⁵ MC38 tumor cells (without Matrigel) subcutaneously in flank. Cell injection volume was 0.1 mL/mouse. Mouse age at start date was 8 to 12 weeks. Pair matches was performed when tumors reached an average size of 100-150 mm³ and treatment began as shown in Table 3. This was Day 1 of the study. Body weights were taken at initiation and then biweekly to the end. Caliper measurements were taken biweekly to the end. Any adverse reactions were reported immediately. Any individual animal with a single observation of >than 25% body weight loss or three consecutive measurements of >20% body weight loss was euthanized. Any group with a mean body weight loss of >20% or >10% mortality stopped dosing; the group were not euthanized, and recovery was allowed. Within a group with >20% weight loss, individuals hitting the individual body weight loss endpoint were euthanized. If the group treatment related body weight loss recovered to within 10% of the original weights, dosing could be resumed at a lower dose or less frequent dosing schedule. Exceptions to non-treatment body weight % recovery were allowed on a case-by-case basis. Tumor volumes were calculated using caliper measurements and followed until end of study. Endpoint were tumor growth delay (TGD). Animals were monitored individually. The endpoint of the experiment were a tumor volume of 1500 mm³ or 40 days, whichever came first. When the endpoint was reached, the animals were euthanized. Results are shown in FIGS. 10-12.

### 8. CONSTRUCTS

The elements of the polypeptide constructs provided in Table 4 contain the abbreviations as follows: “X” refers to a linker. “X” refers to a cleavable linker. Linker 3 refers to a linker that comprises a CTSL-1 substrate motif sequence.

TABLE 4
Exemplary inducible IFN prodrug constructs

| Construct # | Construct Description |
|---|---|
| WW0888 | aHSA-X-hIFNa2b-X-scFv_(Blocker = scFV; X = Linker 3) |
| WW0893 | IFNa2bR2-X-hIFNa2b-X-aHSA_(Blocker = IFN Receptor 2; X = Linker 3) |
| WW0894 | aHSA-X-hIFNa2b-X-IFNa2bR1_(Blocker = IFN Receptor 1; X = Linker 3) |
| WW0895 | IFNa2bR1-X-hIFNa2b-X-aHSA_(Blocker = IFN Receptor 1; X = Linker 3) |
| WW0896 | aHSA-X-IFNa2bR2-X-hIFNa2b-X-IFNa2bR1_(IFN Receptor 1 and 2; X = Linker 3) |
| WW0897 | IFNa2bR2-X-hIFNa2b-X-IFNa2bR1-X-aHSA_(IFN Receptor 1 and 2; X = Linker 3) |
| WW0898 | IFNa2bR2-X-aHSA-X-IFNa2bR1-X-hIFNa2b_(X = Linker 3) |
| WW0889/890 | blocker_heavy-X-hIFNa2b-X-aHSA_(X = Linker 3-1)/Ab_Fab_Light_(kappa) |
| WW0891/892 | aHSA-X-hIFNa2b-X-blocker_heavy_(X = Linker 3-1)/Ab_Fab_Light_(kappa) |
| WW00900 | aHSA-X-mIFNa1-X-mIFNa1R1_(X = Linker3) |
| WW00901 | mIFNa1R1-X-mIFNa1-X-aHSA_(X = Linker3) |
| WW00902 | aHSA-X-mIFNa1R2-X-mIFNa1-X-mIFNa1R1_(X = Linker3) |
| WW00903 | mIFNa1R2-X-mIFNa1-X-mIFNa1R1-X-aHSA_(X = Linker3) |
| WW00904 | mIFNa1R2-X-aHSA-X-mIFNa1R1-X-mIFNa1_(X = Linker3) |
| WW00926 | NOC8_Fab_Heavy |
| WW00930 | scFv-LX-hIFNa2b-X-aHSA_(NOC8_VLVH_X = CTSL1-1) |
| WW00931 | scFv-LX-hIFNa2b-X-aHSA_(NOC8_VHVL_X = CTSL1-1) |
| WW01113 | mIFNa1R2-X-mIFNa1-X-aHSA_(X = Linker3) |
| WW01117 | scFv-LX-aHSA-XL-hIFNa2b_(NOC8_VHVL_X = Linker3) |
| WW01118 | hIFNa2b-X-scFv-X-aHSA_(NOC8_VLVH_X = Linker3) |
| WW01119 | aHSA-X-hIFNa2b-X-scFv_(NOC8_VLVH_X = Linker3) |
| WW01120 | aHSA-X-hIFNa2b-X-Kappa Blocker Fab_(NOC8_VLCL_X = Linker3) |
| WW01121 | hIFNa2b-X-Kappa Blocker Fab-X-aHSA_(NOC8_VLCL_X = Linker3) |
| WW01122 | hIFNa2b-X-Kappa Blocker Fab_(NOC8_VLCL_X = Linker3) |
| WW01123 | Heavy Blocker Fab-X-HSA_(NOC8_VHCH1_X = Linker3) |
| WW01124 | hIFNa2b-X-Kappa Blocker Fab-L-aHSA_(NOC8_VLCL_X = Linker3) |
| WW01125 | Heavy Blocker Fab-L-HSA_(NOC8_VHCH1) |
| WW01126 | scFv-LL-aHSA-XL-hIFNa2b_(NOC8_VHVL_X = Linker3) |
| WW01127 | hIFNa2b-X-scFv-L-aHSA_(NOC8_VLVH_X = Linker3) |
| WW01154 | aHSA-X-hIFNAR2-X-hIFNa2b_(X = Linker3) |
| WW01155 | aHSA-X-hIFNAR1-X-hIFNa2b_(X = Linker3) |
| WW01156 | aHSA-X-mIFNAR2-X-mIFNa1_(X = Linker3) |
| WW01157 | aHSA-X-mIFNAR1-X-mIFNa1_(X = Linker3) |

9. SEQUENCE DISCLOSURE

| SEQ ID | Construct | | |
|---|---|---|---|
| NO: | Code | Description | Sequence |
| 1 | WW00888 | | EVQLVESGGGLVQPGNSLRLSCAASGFTFSKFGM |
| | | | SWVRQAPGKGLEWVSSISGSGRDTLYAESVKGR |
| | | | FTISRDNAKTTLYLQMNSLRPEDTAVYYCTIGGS |
| | | | LSVSSQGTLVTVSSsggpALFKSSFPpgscdlpqthslgsrrt |
| | | | lmllaqmrrislfsclkdrhdfgfpqeefgnqfqkaetipvlhemiqqifnlf |
| | | | stkdssaawdetlldkfytelyqqlndleacviqgvgvtetplmkedsilav |
| | | | rkyfqritlylkekkyspcawevvraeimrsfslstnlqeslrskesggpAL |
| | | | FKSSFPpgsDIQMTQSPSSLSASVGDRVTITCRASQ |
| | | | SVSTSSYSYMHWYQQKPGKAPKVLISYASNLESG |
| | | | VPSRFSGSGSGTDFTLTISSLQPEDFATYYCQHSW |
| | | | GIPRTFGQGTKVEIKggggsggggsggggsEVQLVESG |
| | | | GGLVQPGGSLRLSCATSGYTFTEYIIHWVRQAPG |
| | | | KGLEWVASINPDYDITNYNQRFKGRFTISLDKSK |
| | | | RTAYLQMNSLRAEDTAVYYCASWISDFFDYWG |
| | | | QGTLVTVSS** |
| 2 | WW00889 | | EVQLVESGGGLVQPGGSLRLSCATSGYTFTEYIIH |
| | | | WVRQAPGKGLEWVASINPDYDITNYNQRFKGRF |
| | | | TISLDKSKRTAYLQMNSLRAEDTAVYYCASWISD |
| | | | FFDYWGQGTLVTVSSASTKGPSVFPLAPSSKSTS |
| | | | GGTAALGCLVKDYFPEPVTVSWNSGALTSGVHT |
| | | | FPAVLQSSGLYSLSSVVTVPSSSLGTQTYICNVNH |
| | | | KPSNTKVDKKVEPKSCDKTHTGGGGSGGGGSGG |
| | | | GGSGGGGSsggpALFKSSFPpgscdlpqthslgsrrtlmllaqm |
| | | | rrislfsclkdrhdfgfpqeefgnqfqkaetipvlhemiqqifnlfstkdssaa |
| | | | wdetlldkfytelyqqlndleacviqgvgvtetplmkedsilavrkyfqritl |
| | | | ylkekkyspcawevvraeimrsfslstnlqeslrskesggpALFKSSF |
| | | | PpgsEVQLVESGGGLVQPGNSLRLSCAASGFTFSK |
| | | | FGMSWVRQAPGKGLEWVSSISGSGRDTLYAESV |
| | | | KGRFTISRDNAKTTLYLQMNSLRPEDTAVYYCTI |
| | | | GGSLSVSSQGTLVTVSS** |
| 3 | WW00890 | | DIQMTQSPSSLSASVGDRVTITCRASQSVSTSSYS |
| | | | YMHWYQQKPGKAPKVLISYASNLESGVPSRFSG |
| | | | SGSGTDFTLTISSLQPEDFATYYCQHSWGIPRTFG |
| | | | QGTKVEIKRTVAAPSVFIFPPSDEQLKSGTASVVC |
| | | | LLNNFYPREAKVQWKVDNALQSGNSQESVTEQD |
| | | | SKDSTYSLSSTLTLSKADYEKHKVYACEVTHQGL |
| | | | SSPVTKSFNRGEC** |
| 4 | WW00891 | | EVQLVESGGGLVQPGNSLRLSCAASGFTFSKFGM |
| | | | SWVRQAPGKGLEWVSSISGSGRDTLYAESVKGR |
| | | | FTISRDNAKTTLYLQMNSLRPEDTAVYYCTIGGS |
| | | | LSVSSQGTLVTVSSsggpALFKSSFPpgscdlpqthslgsrrt |
| | | | lmllaqmrrislfsclkdrhdfgfpqeefgnqfqkaetipvlhemiqqifnlf |
| | | | stkdssaawdetlldkfytelyqqlndleacviqgvgvtetplmkedsilav |
| | | | rkyfqritlylkekkyspcawevvraeimrsfslstnlqeslrskesggpAL |
| | | | FKSSFPpgsGGGGSGGGGSGGGGSGGGGSQVQLV |
| | | | QSGAEVKKPGASVKVSCKASGYTFTSYSISWVRQ |
| | | | APGQGLEWMGWISVYNGNTNYAQKFQGRVTMT |
| | | | TDTSTSTAYLELRSLRSDDTAVYYCARDPIAAGY |
| | | | WGQGTLVTVSSASTKGPSVFPLAPSSKSTSGGTA |
| | | | ALGCLVKDYFPEPVTVSWNSGALTSGVHTFPAV |
| | | | LQSSGLYSLSSVVTVPSSSLGTQTYICNVNHKPSN |
| | | | TKVDKKVEPKSC** |
| 5 | WW00892 | | EIVLTQSPGTLSLSPGERATLSCRASQSVSSTYLA |
| | | | WYQQKPGQAPRLLIYGASSRATGIPDRFSGSGSG |
| | | | TDFTLTISRLEPEDFAVYYCQQYGSSPRTFGQGTK |
| | | | VEIKRTVAAPSVFIFPPSDEQLKSGTASVVCLLNN |
| | | | FYPREAKVQWKVDNALQSGNSQESVTEQDSKDS |
| | | | TYSLSSTLTLSKADYEKHKVYACEVTHQGLSSPV |
| | | | TKSFNRGEC** |
| 6 | WW00893 | | ISYDSPDYTDESCTFKISLRNFRSILSWELKNHSIV |
| | | | PTHYTLLYTIMSKPEDLKVVKNCANTTRSFCDLT |
| | | | DEWRSTHEAYVTVLEGFSGNTTLFSCSHNFWLAI |
| | | | DMSFEPPEFEIVGFTNHINVMVKFPSIVEEELQFD |
| | | | LSLVIEEQSEGIVKKHKPEIKGNMSGNFTYIIDKLI |
| | | | PNTNYCVSVYLEHSDEQAVIKSPLKCTLLPPGQES |
| | | | ESAESAKsggpALFKSSFPpgscdlpqthslgsrrtlmllaqmrri |
| | | | slfsclkdrhdfgfpqeefgnqfqkaetipvlhemiqqifnlfstkdssaaw |
| | | | detlldkfytelyqqlndleacviqgvgvtetplmkedsilavrkyfqritlyl |
| | | | kekkyspcawevvraeimrsfslstnlqeslrskesggpALFKSSFPp |
| | | | gsEVQLVESGGGLVQPGNSLRLSCAASGFTFSKFG |
| | | | MSWVRQAPGKGLEWVSSISGSGRDTLYAESVKG |
| | | | RFTISRDNAKTTLYLQMNSLRPEDTAVYYCTIGG |
| | | | SLSVSSQGTLVTVSS |
| 7 | WW00894 | | EVQLVESGGGLVQPGNSLRLSCAASGFTFSKFGM |
| | | | SWVRQAPGKGLEWVSSISGSGRDTLYAESVKGR |
| | | | FTISRDNAKTTLYLQMNSLRPEDTAVYYCTIGGS |
| | | | LSVSSQGTLVTVSSsggpALFKSSFPpgscdlpqthslgsrrt |
| | | | lmllaqmrrislfsclkdrhdfgfpqeefgnqfqkaetipvlhemiqqifnlf |
| | | | stkdssaawdetlldkfytelyqqlndleacviqgvgvtetplmkedsilav |
| | | | rkyfqritlylkekkyspcawevvraeimrsfslstnlqeslrskesggpAL |
| | | | FKSSFPpgsGGGGSGGGGSGGGGSGGGGSKNLKS |
| | | | PQKVEVDIIDDNFILRWNRSDESVGNVTFSFDYQ |
| | | | KTGMDNWIKLSGCQNITSTKCNFSSLKLNVYEEI |
| | | | KLRIRAEKENTSSWYEVDSFTPFRKAQIGPPEVHL |
| | | | EAEDKAIVIHISPGTKDSVMWALDGLSFTYSLVI |
| | | | WKNSSGVEERIENIYSRHKIYKLSPETTYCLKVKA |
| | | | ALLTSWKIGVYSPVHCIKTTVENELPPPENIEVSV |
| | | | QNQNYVLKWDYTYANMTFQVQWLHAFLKRNP |
| | | | GNHLYKWKQIPDCENVKTTQCVFPQNVFQKGIY |
| | | | LLRVQASDGNNTSFWSEEIKFDTEIQ** |
| 8 | WW00895 | | KNLKSPQKVEVDIIDDNFILRWNRSDESVGNVTF |
| | | | SFDYQKTGMDNWIKLSGCQNITSTKCNFSSLKLN |
| | | | VYEEIKLRIRAEKENTSSWYEVDSFTPFRKAQIGP |
| | | | PEVHLEAEDKAIVIHISPGTKDSVMWALDGLSFT |
| | | | YSLVIWKNSSGVEERIENIYSRHKIYKLSPETTYC |
| | | | LKVKAALLTSWKIGVYSPVHCIKTTVENELPPPE |
| | | | NIEVSVQNQNYVLKWDYTYANMTFQVQWLHAF |
| | | | LKRNPGNHLYKWKQIPDCENVKTTQCVFPQNVF |
| | | | QKGIYLLRVQASDGNNTSFWSEEIKFDTEIQGGG |
| | | | GSsggpALFKSSFPpgscdlpqthslgsrrtlmllaqmrrislfsclkd |
| | | | rhdfgfpqeefgnqfqkaetipvlhemiqqifnlfstkdssaawdetlldkf |
| | | | ytelyqqlndleacviqgvgvtetplmkedsilavrkyfqritlylkekkys |
| | | | pcawevvraeimrsfslstnlqeslrskesggpALFKSSFPpgsEVQ |
| | | | LVESGGGLVQPGNSLRLSCAASGFTFSKFGMSW |
| | | | VRQAPGKGLEWVSSISGSGRDTLYAESVKGRFTI |
| | | | SRDNAKTTLYLQMNSLRPEDTAVYYCTIGGSLSV |
| | | | SSQGTLVTVSS** |
| 9 | WW00896 | | EVQLVESGGGLVQPGNSLRLSCAASGFTFSKFGM |
| | | | SWVRQAPGKGLEWVSSISGSGRDTLYAESVKGR |
| | | | FTISRDNAKTTLYLQMNSLRPEDTAVYYCTIGGS |
| | | | LSVSSQGTLVTVSSsggpALFKSSFPpgsISYDSPDYT |
| | | | DESCTFKISLRNFRSILSWELKNHSIVPTHYTLLYT |
| | | | IMSKPEDLKVVKNCANTTRSFCDLTDEWRSTHE |
| | | | AYVTVLEGFSGNTTLFSCSHNFWLAIDMSFEPPEF |
| | | | EIVGFTNHINVMVKFPSIVEEELQFDLSLVIEEQSE |
| | | | GIVKKHKPEIKGNMSGNFTYIIDKLIPNTNYCVSV |
| | | | YLEHSDEQAVIKSPLKCTLLPPGQESESAESAKGG |
| | | | GGSGGGGSGGGGSGGGGSsggpALFKSSFPpgscdlp |
| | | | qthslgsrrtlmllaqmrrislfsclkdrhdfgfpqeefgnqfqkaetipvlhe |
| | | | miqqifnlfstkdssaawdetlldkfytelyqqlndleacviqgvgvtetpl |
| | | | mkedsilavrkyfqritlylkekkyspcawevvraeimrsfslstnlqeslrs |
| | | | kesggpALFKSSFPpgsGGGGSGGGGSGGGGSGGGG |
| | | | SKNLKSPQKVEVDIIDDNFILRWNRSDESVGNVT |
| | | | FSFDYQKTGMDNWIKLSGCQNITSTKCNFSSLKL |
| | | | NVYEEIKLRIRAEKENTSSWYEVDSFTPFRKAQIG |
| | | | PPEVHLEAEDKAIVIHISPGTKDSVMWALDGLSFT |
| | | | YSLVIWKNSSGVEERIENIYSRHKIYKLSPETTYC |
| | | | LKVKAALLTSWKIGVYSPVHCIKTTVENELPPPE |
| | | | NIEVSVQNQNYVLKWDYTYANMTFQVQWLHAF |
| | | | LKRNPGNHLYKWKQIPDCENVKTTQCVFPQNVF |
| | | | QKGIYLLRVQASDGNNTSFWSEEIKFDTEIQ** |
| 10 | WW00897 | | ISYDSPDYTDESCTFKISLRNFRSILSWELKNHSIV |
| | | | PTHYTLLYTIMSKPEDLKVVKNCANTTRSFCDLT |
| | | | DEWRSTHEAYVTVLEGFSGNTTLFSCSHNFWLAI |
| | | | DMSFEPPEFEIVGFTNHINVMVKFPSIVEEELQFD |
| | | | LSLVIEEQSEGIVKKHKPEIKGNMSGNFTYIIDKLI |
| | | | PNTNYCVSVYLEHSDEQAVIKSPLKCTLLPPGQES |
| | | | ESAESAKGGGGSGGGGSGGGGSGGGGSsggpALF |
| | | | KSSFPpgscdlpqthslgsrrtlmllaqmrrislfsclkdrhdfgfpqeefg |
| | | | nqfqkaetipvlhemiqqifnlfstkdssaawdetlldkfytelyqqlndlea |
| | | | cviqgvgvtetplmkedsilavrkyfqritlylkekkyspcawevvraeim |
| | | | rsfslstnlqeslrskesggpALFKSSFPpgsGGGGSGGGGSG |
| | | | GGGSGGGGSKNLKSPQKVEVDIIDDNFILRWNRS |
| | | | DESVGNVTFSFDYQKTGMDNWIKLSGCQNITSTK |
| | | | CNFSSLKLNVYEEIKLRIRAEKENTSSWYEVDSFT |
| | | | PFRKAQIGPPEVHLEAEDKAIVIHISPGTKDSVMW |
| | | | ALDGLSFTYSLVIWKNSSGVEERIENIYSRHKIYK |
| | | | LSPETTYCLKVKAALLTSWKIGVYSPVHCIKTTV |
| | | | ENELPPPENIEVSVQNQNYVLKWDYTYANMTFQ |
| | | | VQWLHAFLKRNPGNHLYKWKQIPDCENVKTTQ |
| | | | CVFPQNVFQKGIYLLRVQASDGNNTSFWSEEIKF |
| | | | DTEIQsggpALFKSSFPpgsEVQLVESGGGLVQPGNS |
| | | | LRLSCAASGFTFSKFGMSWVRQAPGKGLEWVSSI |
| | | | SGSGRDTLYAESVKGRFTISRDNAKTTLYLQMNS |
| | | | LRPEDTAVYYCTIGGSLSVSSQGTLVTVSS** |
| 11 | WW00898 | | ISYDSPDYTDESCTFKISLRNFRSILSWELKNHSIV |
| | | | PTHYTLLYTIMSKPEDLKVVKNCANTTRSFCDLT |
| | | | DEWRSTHEAYVTVLEGFSGNTTLFSCSHNFWLAI |
| | | | DMSFEPPEFEIVGFTNHINVMVKFPSIVEEELQFD |
| | | | LSLVIEEQSEGIVKKHKPEIKGNMSGNFTYIIDKLI |
| | | | PNTNYCVSVYLEHSDEQAVIKSPLKCTLLPPGQES |
| | | | ESAESAKGGGGSGGGGSGGGGSsggpALFKSSFPp |
| | | | gsEVQLVESGGGLVQPGNSLRLSCAASGFTFSKFG |
| | | | MSWVRQAPGKGLEWVSSISGSGRDTLYAESVKG |
| | | | RFTISRDNAKTTLYLQMNSLRPEDTAVYYCTIGG |
| | | | SLSVSSQGTLVTVSSsggpALFKSSFPpgsGGGGSGG |
| | | | GGSGGGGSKNLKSPQKVEVDIIDDNFILRWNRSD |
| | | | ESVGNVTFSFDYQKTGMDNWIKLSGCQNITSTKC |
| | | | NFSSLKLNVYEEIKLRIRAEKENTSSWYEVDSFTP |
| | | | FRKAQIGPPEVHLEAEDKAIVIHISPGTKDSVMW |
| | | | ALDGLSFTYSLVIWKNSSGVEERIENIYSRHKIYK |
| | | | LSPETTYCLKVKAALLTSWKIGVYSPVHCIKTTV |
| | | | ENELPPPENIEVSVQNQNYVLKWDYTYANMTFQ |
| | | | VQWLHAFLKRNPGNHLYKWKQIPDCENVKTTQ |
| | | | CVFPQNVFQKGIYLLRVQASDGNNTSFWSEEIKF |
| | | | DTEIQsggpALFKSSFPpgscdlpqthslgsrrtlmllaqmrrislfs |
| | | | clkdrhdfgfpqeefgnqfqkaetipvlhemiqqifnlfstkdssaawdetl |
| | | | ldkfytelyqqlndleacviqgvgvtetplmkedsilavrkyfqritlylkek |
| | | | kyspcawevvraeimrsfslstnlqeslrske** |
| 12 | WW00900 | | EVQLVESGGGLVQPGNSLRLSCAASGFTFSKFG |
| | | | MSWVRQAPGKGLEWVSSISGSGRDTLYAESV |
| | | | KGRFTISRDNAKTTLYLQMNSLRPEDTAVYYC |
| | | | TIGGSLSVSSQGTLVTVSSsggpALFKSSFPpgscdl |
| | | | pqthnlrnkraltllvqmrrlsplsclkdrkdfgfpqekvdaqqikkaqa |
| | | | ipvlseltqqilniftskdssaawnttlldsfcndlhqqlndlqgclmqqv |
| | | | gvqefpltqedallavrkyfhritvylrekkhspcawevvraevwrals |
| | | | ssanvlgrlreeksggpALFKSSFPpgsGGGGSGGGGSG |
| | | | GGGSGGGGSENLKPPENIDVYIIDDNYTLKWSS |
| | | | HGESMGSVTFSAEYRTKDEAKWLKVPECQHT |
| | | | TTTKC |
| | | | EFSLLDTNVYIKTQFRVRAEEGNSTSSWNEVDP |
| | | | FIPFYTAHMSPPEVRLEAEDKAILVHI |
| | | | SPPGQDGNMWALEKPSFSYTIRIWQKSSSDKK |
| | | | TINSTYYVEKIPELLPETTYCLEVKAIH |
| | | | PSLKKHSNYSTVQCISTTVANKMPVPGNLQVD |
| | | | AQGKSYVLKWDYIASADVLFRAQWLPGY |
| | | | SKSSSGSRSDKWKPIPTCANVQTTHCVFSQDTV |
| | | | YTGTFFLHVQASEGNHTSFWSEEKFID |
| | | | SQKH** |
| 13 | WW00901 | | ENLKPPENIDVYIIDDNYTLKWSSHGESMGSVT |
| | | | FSAEYRTKDEAKWLKVPECQHTTTTKC |
| | | | EFSLLDTNVYIKTQFRVRAEEGNSTSSWNEVDP |
| | | | FIPFYTAHMSPPEVRLEAEDKAILVHI |
| | | | SPPGQDGNMWALEKPSFSYTIRIWQKSSSDKK |
| | | | TINSTYYVEKIPELLPETTYCLEVKAIH |
| | | | PSLKKHSNYSTVQCISTTVANKMPVPGNLQVD |
| | | | AQGKSYVLKWDYIASADVLFRAQWLPGY |
| | | | SKSSSGSRSDKWKPIPTCANVQTTHCVFSQDTV |
| | | | YTGTFFLHVQASEGNHTSFWSEEKFID |
| | | | SQKHGGGGSsggpALFKSSFPpgscdlpqthnlrnkraltll |
| | | | vqmrrlsplsclkdrkdfgfpqekvdaqqikkaqaipvlseltqqilnift |
| | | | skdssaawnttlldsfcndlhqqlndlqgclmqqvgvqefpltqedall |
| | | | avrkyfhritvylrekkhspcawevvraevwralsssanvlgrlreeks |
| | | | ggpALFKSSFPpgsEVQLVESGGGLVQPGNSLRL |
| | | | SCAASGFTFSKFGMSWVRQAPGKGLEWVSSIS |
| | | | GSGRDTLYAESVKGRFTISRDNAKTTLYLQMN |
| | | | SLRPEDTAVYYCTIGGSLSVSSQGTLVTVSS** |
| 14 | WW00902 | | EVQLVESGGGLVQPGNSLRLSCAASGFTFSKFG |
| | | | MSWVRQAPGKGLEWVSSISGSGRDTLYAESV |
| | | | KGRFTISRDNAKTTLYLQMNSLRPEDTAVYYC |
| | | | TIGGSLSVSSQGTLVTVSSsggpALFKSSFPpgsSL |
| | | | ETITPSAFDGYPDEPCTINITIRNSRLILSWELEN |
| | | | KSGPPANYTLWYTVMSKDENLTK |
| | | | VKNCSDTTKSSCDVTDKWLEGMESYVVAIVIV |
| | | | HRGDLTVCRCSDYIVPANAPLEPPEFEI |
| | | | VGFTDHINVTMEFPPVTSKIIQEKMKTTPFVIKE |
| | | | QIGDSVRKKHEPKVNNVTGNFTFVLR |
| | | | DLLPKTNYCVSLYFDDDPAIKSPLKCIVLQPGQ |
| | | | ESGLSESAGGGGSGGGGSGGGGSGGGGSsggpA |
| | | | LFKSSFPpgscdlpqthnlrnkraltllvqmrrlsplsclkdrkdfgf |
| | | | pqekvdaqqikkaqaipvlseltqqilniftskdssaawnttlldsfcndl |
| | | | hqqlndlqgclmqqvgvqefpltqedallavrkyfhritvylrekkhsp |
| | | | cawevvraevwralsssanvlgrlreeksggpALFKSSFPpgsG |
| | | | GGGSGGGGSGGGGSGGGGSENLKPPENIDVYII |
| | | | DDNYTLKWSSHGESMGSVTFSAEYRTKDEAK |
| | | | WLKVPECQHTTTTKC |
| | | | EFSLLDTNVYIKTQFRVRAEEGNSTSSWNEVDP |
| | | | FIPFYTAHMSPPEVRLEAEDKAILVHI |
| | | | SPPGQDGNMWALEKPSFSYTIRIWQKSSSDKK |
| | | | TINSTYYVEKIPELLPETTYCLEVKAIH |
| | | | PSLKKHSNYSTVQCISTTVANKMPVPGNLQVD |
| | | | AQGKSYVLKWDYIASADVLFRAQWLPGY |
| | | | SKSSSGSRSDKWKPIPTCANVQTTHCVFSQDTV |
| | | | YTGTFFLHVQASEGNHTSFWSEEKFID |
| | | | SQKH** |
| 15 | WW00903 | | SLETITPSAFDGYPDEPCTINITIRNSRLILSWELE |
| | | | NKSGPPANYTLWYTVMSKDENLTK |
| | | | VKNCSDTTKSSCDVTDKWLEGMESYVVAIVIV |
| | | | HRGDLTVCRCSDYIVPANAPLEPPEFEI |
| | | | VGFTDHINVTMEFPPVTSKIIQEKMKTTPFVIKE |
| | | | QIGDSVRKKHEPKVNNVTGNFTFVLR |
| | | | DLLPKTNYCVSLYFDDDPAIKSPLKCIVLQPGQ |
| | | | ESGLSESAGGGGSGGGGSGGGGSGGGGSsggpA |
| | | | LFKSSFPpgscdlpqthnlrnkraltllvqmrrlsplsclkdrkdfgf |
| | | | pqekvdaqqikkaqaipvlseltqqilniftskdssaawnttlldsfcndl |
| | | | hqqlndlqgclmqqvgvqefpltqedallavrkyfhritvylrekkhsp |
| | | | cawevvraevwralsssanvlgrlreeksggpALFKSSFPpgsG |
| | | | GGGSGGGGSGGGGSGGGGSENLKPPENIDVYII |
| | | | DDNYTLKWSSHGESMGSVTFSAEYRTKDEAK |
| | | | WLKVPECQHTTTTKC |
| | | | EFSLLDTNVYIKTQFRVRAEEGNSTSSWNEVDP |
| | | | FIPFYTAHMSPPEVRLEAEDKAILVHI |
| | | | SPPGQDGNMWALEKPSFSYTIRIWQKSSSDKK |
| | | | TINSTYYVEKIPELLPETTYCLEVKAIH |
| | | | PSLKKHSNYSTVQCISTTVANKMPVPGNLQVD |
| | | | AQGKSYVLKWDYIASADVLFRAQWLPGY |
| | | | SKSSSGSRSDKWKPIPTCANVQTTHCVFSQDTV |
| | | | YTGTFFLHVQASEGNHTSFWSEEKFID |
| | | | SQKHsggpALFKSSFPpgsEVQLVESGGGLVQPG |
| | | | NSLRLSCAASGFTFSKFGMSWVRQAPGKGLE |
| | | | WVSSISGSGRDTLYAESVKGRFTISRDNAKTTL |
| | | | YLQMNSLRPEDTAVYYCTIGGSLSVSSQGTLV |
| | | | TVSS** |
| 16 | WW00904 | | SLETITPSAFDGYPDEPCTINITIRNSRLILSWELE |
| | | | NKSGPPANYTLWYTVMSKDENLTK |
| | | | VKNCSDTTKSSCDVTDKWLEGMESYVVAIVIV |
| | | | HRGDLTVCRCSDYIVPANAPLEPPEFEI |
| | | | VGFTDHINVTMEFPPVTSKIIQEKMKTTPFVIKE |
| | | | QIGDSVRKKHEPKVNNVTGNFTFVLR |
| | | | DLLPKTNYCVSLYFDDDPAIKSPLKCIVLQPGQ |
| | | | ESGLSESAGGGGSGGGGSGGGGSsggpALFKSSF |
| | | | PpgsEVQLVESGGGLVQPGNSLRLSCAASGFTFS |
| | | | KFGMSWVRQAPGKGLEWVSSISGSGRDTLYA |
| | | | ESVKGRFTISRDNAKTTLYLQMNSLRPEDTAV |
| | | | YYCTIGGSLSVSSQGTLVTVSSsggpALFKSSFPp |
| | | | gsGGGGSGGGGSGGGGSENLKPPENIDVYIIDD |
| | | | NYTLKWSSHGESMGSVTFSAEYRTKDEAKWL |
| | | | KVPECQHTTTTKC |
| | | | EFSLLDTNVYIKTQFRVRAEEGNSTSSWNEVDP |
| | | | FIPFYTAHMSPPEVRLEAEDKAILVHI |
| | | | SPPGQDGNMWALEKPSFSYTIRIWQKSSSDKK |
| | | | TINSTYYVEKIPELLPETTYCLEVKAIH |
| | | | PSLKKHSNYSTVQCISTTVANKMPVPGNLQVD |
| | | | AQGKSYVLKWDYIASADVLFRAQWLPGY |
| | | | SKSSSGSRSDKWKPIPTCANVQTTHCVFSQDTV |
| | | | YTGTFFLHVQASEGNHTSFWSEEKFID |
| | | | SQKHsggpALFKSSFPpgscdlpqthnlrnkraltllvqmrrlsp |
| | | | lsclkdrkdfgfpqekvdaqqikkaqaipvlseltqqilniftskdssaa |
| | | | wnttlldsfcndlhqqlndlqgclmqqvgvqefpltqedallavrkyfh |
| | | | ritvylrekkhspcawevvraevwralsssanvlgrlreek** |
| 17 | WW00926 | | EVQLVESGGGLVQPGGSLRLSCATSGYTFTEYI |
| | | | IHWVRQAPGKGLEWVASINPDYDITNYNQRFK |
| | | | GRFTISLDKSKRTAYLQMNSLRAEDTAVYYCA |
| | | | SWISDFFDYWGQGTLVTVSSASTKGPSVFPLAP |
| | | | SSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTHH |
| | | | HHHH** |
| 18 | WW00930 | | DIQMTQSPSSLSASVGDRVTITCRASQSVSTSSY |
| | | | SYMHWYQQKPGKAPKVLISYASNLESGVPSRF |
| | | | SGSGSGTDFTLTISSLQPEDFATYYCQHSWGIPR |
| | | | TFGQGTKVEIKggggsggggsggggsEVQLVESGGG |
| | | | LVQPGGSLRLSCATSGYTFTEYIIHWVRQAPGK |
| | | | GLEWVASINPDYDITNYNQRFKGRFTISLDKSK |
| | | | RTAYLQMNSLRAEDTAVYYCASWISDFFDYW |
| | | | GQGTLVTVSSGGGGSGGGGSGGGGSsggpALFK |
| | | | SSFPpgscdlpqthslgsrrtlmllaqmrrislfsclkdrhdfgfpqeef |
| | | | gnqfqkaetipvlhemiqqifnlfstkdssaawdetlldkfytelyqqln |
| | | | dleacviqgvgvtetplmkedsilavrkyfqritlylkekkyspcawev |
| | | | vraeimrsfslstnlqeslrskesggpALFKSSFPpgsEVQLVE |
| | | | SGGGLVQPGNSLRLSCAASGFTFSKFGMSWVR |
| | | | QAPGKGLEWVSSISGSGRDTLYAESVKGRFTIS |
| | | | RDNAKTTLYLQMNSLRPEDTAVYYCTIGGSLS |
| | | | VSSQGTLVTVSS** |
| 19 | WW00931 | | EVQLVESGGGLVQPGGSLRLSCATSGYTFTEYI |
| | | | IHWVRQAPGKGLEWVASINPDYDITNYNQRFK |
| | | | GRFTISLDKSKRTAYLQMNSLRAEDTAVYYCA |
| | | | SWISDFFDYWGQGTLVTVSSggggsggggggggsDI |
| | | | QMTQSPSSLSASVGDRVTITCRASQSVSTSSYS |
| | | | YMHWYQQKPGKAPKVLISYASNLESGVPSRFS |
| | | | GSGSGTDFTLTISSLQPEDFATYYCQHSWGIPR |
| | | | TFGQGTKVEIKGGGGSGGGGSGGGGSsggpALF |
| | | | KSSFPpgscdlpqthslgsrrtlmllaqmrrislfsclkdrhdfgfpqe |
| | | | efgnqfqkaetipvlhemiqqifnlfstkdssaawdetlldkfytelyqq |
| | | | lndleacviqgvgvtetplmkedsilavrkyfqritlylkekkyspcaw |
| | | | evvraeimrsfslstnlqeslrskesggpALFKSSFPpgsEVQL |
| | | | VESGGGLVQPGNSLRLSCAASGFTFSKFGMSW |
| | | | VRQAPGKGLEWVSSISGSGRDTLYAESVKGRF |
| | | | TISRDNAKTTLYLQMNSLRPEDTAVYYCTIGGS |
| | | | LSVSSQGTLVTVSS** |
| 20 | WW01113 | | SLETITPSAFDGYPDEPCTINITIRNSRLILSWELE |
| | | | NKSGPPANYTLWYTVMSKDENLTKVKNCSDT |
| | | | TKSSCDVTDKWLEGMESYVVAIVIVHRGDLTV |
| | | | CRCSDYIVPANAPLEPPEFEIVGFTDHINVTMEF |
| | | | PPVTSKIIQEKMKTTPFVIKEQIGDSVRKKHEPK |
| | | | VNNVTGNFTFVLRDLLPKTNYCVSLYFDDDPA |
| | | | IKSPLKCIVLQPGQESGLSESAGGGGSGGGGSsg |
| | | | gpALFKSSFPpgscdlpqthnlrnkraltllvqmrrlsplsclkdrk |
| | | | dfgfpqekvdaqqikkaqaipvlseltqqilniftskdssaawnttlldsf |
| | | | cndlhqqlndlqgclmqqvgvqefpltqedallavrkyfhritvylrek |
| | | | khspcawevvraevwralsssanvlgrlreeksggpALFKSSFPp |
| | | | gsEVQLVESGGGLVQPGNSLRLSCAASGFTFSK |
| | | | FGMSWVRQAPGKGLEWVSSISGSGRDTLYAES |
| | | | VKGRFTISRDNAKTTLYLQMNSLRPEDTAVYY |
| | | | CTIGGSLSVSSQGTLVTVSS** |
| 21 | WW01117 | | EVQLVESGGGLVQPGGSLRLSCATSGYTFTEYI |
| | | | IHWVRQAPGKGLEWVASINPDYDITNYNQRFK |
| | | | GRFTISLDKSKRTAYLQMNSLRAEDTAVYYCA |
| | | | SWISDFFDYWGQGTLVTVSSggggsggggsggggsgg |
| | | | ggsDIQMTQSPSSLSASVGDRVTITCRASQSVST |
| | | | SSYSYMHWYQQKPGKAPKVLISYASNLESGVP |
| | | | SRFSGSGSGTDFTLTISSLQPEDFATYYCQHSW |
| | | | GIPRTFGQGTKVEIKGGGGSGGGGSsggpALFKS |
| | | | SFPpgsEVQLVESGGGLVQPGNSLRLSCAASGFT |
| | | | FSKFGMSWVRQAPGKGLEWVSSISGSGRDTLY |
| | | | AESVKGRFTISRDNAKTTLYLQMNSLRPEDTA |
| | | | VYYCTIGGSLSVSSQGTLVTVSSsggpALFKSSFP |
| | | | pgsGGGGSGGGGScdlpqthslgsrrtlmllaqmrrislfsclkd |
| | | | rhdfgfpqeefgnqfqkaetipvlhemiqqifnlfstkdssaawdetlld |
| | | | kfytelyqqlndleacviqgvgvtetplmkedsilavrkyfqritlylkek |
| | | | kyspcawevvraeimrsfslstnlqeslrske** |
| 22 | WW01118 | | cdlpqthslgsrrtlmllaqmrrislfsclkdrhdfgfpqeefgnqfqkae |
| | | | tipvlhemiqqifnlfstkdssaawdetlldkfytelyqqlndleacviqg |
| | | | vgvtetplmkedsilavrkyfqritlylkekkyspcawevvraeimrsf |
| | | | slstnlqeslrskesggpALFKSSFPpgsDIQMTQSPSSLS |
| | | | ASVGDRVTITCRASQSVSTSSYSYMHWYQQKP |
| | | | GKAPKVLISYASNLESGVPSRFSGSGSGTDFTL |
| | | | TISSLQPEDFATYYCQHSWGIPRTFGQGTKVEI |
| | | | KggggsggggsggggsggggsEVQLVESGGGLVQPGG |
| | | | SLRLSCATSGYTFTEYIIHWVRQAPGKGLEWV |
| | | | ASINPDYDITNYNQRFKGRFTISLDKSKRTAYL |
| | | | QMNSLRAEDTAVYYCASWISDFFDYWGQGTL |
| | | | VTVSSsggpALFKSSFPpgsEVQLVESGGGLVQPG |
| | | | NSLRLSCAASGFTFSKFGMSWVRQAPGKGLE |
| | | | WVSSISGSGRDTLYAESVKGRFTISRDNAKTTL |
| | | | YLQMNSLRPEDTAVYYCTIGGSLSVSSQGTLV |
| | | | TVSS** |
| 23 | WW01119 | | EVQLVESGGGLVQPGNSLRLSCAASGFTFSKFG |
| | | | MSWVRQAPGKGLEWVSSISGSGRDTLYAESV |
| | | | KGRFTISRDNAKTTLYLQMNSLRPEDTAVYYC |
| | | | TIGGSLSVSSQGTLVTVSSsggpALFKSSFPpgscdl |
| | | | pqthslgsrrtlmllaqmrrislfsclkdrhdfgfpqeefgnqfqkaetip |
| | | | vlhemiqqifnlfstkdssaawdetlldkfytelyqqlndleacviqgvg |
| | | | vtetplmkedsilavrkyfqritlylkekkyspcawevvraeimrsfsls |
| | | | tnlqeslrskesggpALFKSSFPpgsDIQMTQSPSSLSAS |
| | | | VGDRVTITCRASQSVSTSSYSYMHWYQQKPGK |
| | | | APKVLISYASNLESGVPSRFSGSGSGTDFTLTIS |
| | | | SLQPEDFATYYCQHSWGIPRTFGQGTKVEIKgg |
| | | | ggsggggsggggsggggsEVQLVESGGGLVQPGGSLR |
| | | | LSCATSGYTFTEYIIHWVRQAPGKGLEWVASIN |
| | | | PDYDITNYNQRFKGRFTISLDKSKRTAYLQMN |
| | | | SLRAEDTAVYYCASWISDFFDYWGQGTLVTVS |
| | | | S** |
| 24 | WW01120 | | EVQLVESGGGLVQPGNSLRLSCAASGFTFSKFG |
| | | | MSWVRQAPGKGLEWVSSISGSGRDTLYAESV |
| | | | KGRFTISRDNAKTTLYLQMNSLRPEDTAVYYC |
| | | | TIGGSLSVSSQGTLVTVSSsggpALFKSSFPpgscdl |
| | | | pqthslgsrrtlmllaqmrrislfsclkdrhdfgfpqeefgnqfqkaetip |
| | | | vlhemiqqifnlfstkdssaawdetlldkfytelyqqlndleacviqgvg |
| | | | vtetplmkedsilavrkyfqritlylkekkyspcawevvraeimrsfsls |
| | | | tnlqeslrskesggpALFKSSFPpgsDIQMTQSPSSLSAS |
| | | | VGDRVTITCRASQSVSTSSYSYMHWYQQKPGK |
| | | | APKVLISYASNLESGVPSRFSGSGSGTDFTLTIS |
| | | | SLQPEDFATYYCQHSWGIPRTFGQGTKVEIKRT |
| | | | VAAPSVFIFPPSDEQLKSGTASVVCLLNNFYPR |
| | | | EAKVQWKVDNALQSGNSQESVTEQDSKDSTY |
| | | | SLSSTLTLSKADYEKHKVYACEVTHQGLSSPV |
| | | | TKSFNRGEC** |
| 25 | WW01121 | | cdlpqthslgsrrtlmllaqmrrislfsclkdrhdfgfpqeefgnqfqkae |
| | | | tipvlhemiqqifnlfstkdssaawdetlldkfytelyqqlndleacviqg |
| | | | vgvtetplmkedsilavrkyfqritlylkekkyspcawevvraeimrsf |
| | | | slstnlqeslrskesggpALFKSSFPpgsDIQMTQSPSSLS |
| | | | ASVGDRVTITCRASQSVSTSSYSYMHWYQQKP |
| | | | GKAPKVLISYASNLESGVPSRFSGSGSGTDFTL |
| | | | TISSLQPEDFATYYCQHSWGIPRTFGQGTKVEI |
| | | | KRTVAAPSVFIFPPSDEQLKSGTASVVCLLNNF |
| | | | YPREAKVQWKVDNALQSGNSQESVTEQDSKD |
| | | | STYSLSSTLTLSKADYEKHKVYACEVTHQGLS |
| | | | SPVTKSFNRGECsggpALFKSSFPpgsEVQLVESG |
| | | | GGLVQPGNSLRLSCAASGFTFSKFGMSWVRQA |
| | | | PGKGLEWVSSISGSGRDTLYAESVKGRFTISRD |
| | | | NAKTTLYLQMNSLRPEDTAVYYCTIGGSLSVS |
| | | | SQGTLVTVSS** |
| 26 | WW01122 | | cdlpqthslgsrrtlmllaqmrrislfsclkdrhdfgfpqeefgnqfqkae |
| | | | tipvlhemiqqifnlfstkdssaawdetlldkfytelyqqlndleacviqg |
| | | | vgvtetplmkedsilavrkyfqritlylkekkyspcawevvraeimrsf |
| | | | slstnlqeslrskesggpALFKSSFPpgsDIQMTQSPSSLS |
| | | | ASVGDRVTITCRASQSVSTSSYSYMHWYQQKP |
| | | | GKAPKVLISYASNLESGVPSRFSGSGSGTDFTL |
| | | | TISSLQPEDFATYYCQHSWGIPRTFGQGTKVEI |
| | | | KRTVAAPSVFIFPPSDEQLKSGTASVVCLLNNF |
| | | | YPREAKVQWKVDNALQSGNSQESVTEQDSKD |
| | | | STYSLSSTLTLSKADYEKHKVYACEVTHQGLS |
| | | | SPVTKSFNRGEC** |
| 27 | WW01123 | | EVQLVESGGGLVQPGGSLRLSCATSGYTFTEYI |
| | | | IHWVRQAPGKGLEWVASINPDYDITNYNQRFK |
| | | | GRFTISLDKSKRTAYLQMNSLRAEDTAVYYCA |
| | | | SWISDFFDYWGQGTLVTVSSASTKGPSVFPLAP |
| | | | SSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTsgg |
| | | | pALFKSSFPpgsEVQLVESGGGLVQPGNSLRLSC |
| | | | AASGFTFSKFGMSWVRQAPGKGLEWVSSISGS |
| | | | GRDTLYAESVKGRFTISRDNAKTTLYLQMNSL |
| | | | RPEDTAVYYCTIGGSLSVSSQGTLVTVSS** |
| 28 | WW01124 | | cdlpqthslgsrrtlmllaqmrrislfsclkdrhdfgfpqeefgnqfqkae |
| | | | tipvlhemiqqifnlfstkdssaawdetlldkfytelyqqlndleacviqg |
| | | | vgvtetplmkedsilavrkyfqritlylkekkyspcawevvraeimrsf |
| | | | slstnlqeslrskesggpALFKSSFPpgsDIQMTQSPSSLS |
| | | | ASVGDRVTITCRASQSVSTSSYSYMHWYQQKP |
| | | | GKAPKVLISYASNLESGVPSRFSGSGSGTDFTL |
| | | | TISSLQPEDFATYYCQHSWGIPRTFGQGTKVEI |
| | | | KRTVAAPSVFIFPPSDEQLKSGTASVVCLLNNF |
| | | | YPREAKVQWKVDNALQSGNSQESVTEQDSKD |
| | | | STYSLSSTLTLSKADYEKHKVYACEVTHQGLS |
| | | | SPVTKSFNRGECggggsggggsggggsEVQLVESGG |
| | | | GLVQPGNSLRLSCAASGFTFSKFGMSWVRQAP |
| | | | GKGLEWVSSISGSGRDTLYAESVKGRFTISRDN |
| | | | AKTTLYLQMNSLRPEDTAVYYCTIGGSLSVSS |
| | | | QGTLVTVSS** |
| 29 | WW01125 | | EVQLVESGGGLVQPGGSLRLSCATSGYTFTEYI |
| | | | IHWVRQAPGKGLEWVASINPDYDITNYNQRFK |
| | | | GRFTISLDKSKRTAYLQMNSLRAEDTAVYYCA |
| | | | SWISDFFDYWGQGTLVTVSSASTKGPSVFPLAP |
| | | | SSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTggg |
| | | | gsggggsggggsEVQLVESGGGLVQPGNSLRLSCA |
| | | | ASGFTFSKFGMSWVRQAPGKGLEWVSSISGSG |
| | | | RDTLYAESVKGRFTISRDNAKTTLYLQMNSLR |
| | | | PEDTAVYYCTIGGSLSVSSQGTLVTVSS** |
| 30 | WW01126 | | EVQLVESGGGLVQPGGSLRLSCATSGYTFTEYI |
| | | | IHWVRQAPGKGLEWVASINPDYDITNYNQRFK |
| | | | GRFTISLDKSKRTAYLQMNSLRAEDTAVYYCA |
| | | | SWISDFFDYWGQGTLVTVSSggggsggggsggggsgg |
| | | | ggsDIQMTQSPSSLSASVGDRVTITCRASQSVST |
| | | | SSYSYMHWYQQKPGKAPKVLISYASNLESGVP |
| | | | SRFSGSGSGTDFTLTISSLQPEDFATYYCQHSW |
| | | | GIPRTFGQGTKVEIKGGGGSGGGGSggggsggggs |
| | | | ggggsEVQLVESGGGLVQPGNSLRLSCAASGFTF |
| | | | SKFGMSWVRQAPGKGLEWVSSISGSGRDTLYA |
| | | | ESVKGRFTISRDNAKTTLYLQMNSLRPEDTAV |
| | | | YYCTIGGSLSVSSQGTLVTVSSsggpALFKSSFPp |
| | | | gsGGGGSGGGGScdlpqthslgsrrtlmllaqmrrislfsclkdr |
| | | | hdfgfpqeefgnqfqkaetipvlhemiqqifnlfstkdssaawdetlld |
| | | | kfytelyqqlndleacviqgvgvtetplmkedsilavrkyfqritlylkek |
| | | | kyspcawevvraeimrsfslstnlqeslrske** |
| 31 | WW01127 | | cdlpqthslgsrrtlmllaqmrrislfsclkdrhdfgfpqeefgnqfqkae |
| | | | tipvlhemiqqifnlfstkdssaawdetlldkfytelyqqlndleacviqg |
| | | | vgvtetplmkedsilavrkyfqritlylkekkyspcawevvraeimrsf |
| | | | slstnlqeslrskesggpALFKSSFPpgsDIQMTQSPSSLS |
| | | | ASVGDRVTITCRASQSVSTSSYSYMHWYQQKP |
| | | | GKAPKVLISYASNLESGVPSRFSGSGSGTDFTL |
| | | | TISSLQPEDFATYYCQHSWGIPRTFGQGTKVEI |
| | | | KggggsggggsggggsggggsEVQLVESGGGLVQPGG |
| | | | SLRLSCATSGYTFTEYIIHWVRQAPGKGLEWV |
| | | | ASINPDYDITNYNQRFKGRFTISLDKSKRTAYL |
| | | | QMNSLRAEDTAVYYCASWISDFFDYWGQGTL |
| | | | VTVSSggggsggggsggggsEVQLVESGGGLVQPGN |
| | | | SLRLSCAASGFTFSKFGMSWVRQAPGKGLEW |
| | | | VSSISGSGRDTLYAESVKGRFTISRDNAKTTLY |
| | | | LQMNSLRPEDTAVYYCTIGGSLSVSSQGTLVT |
| | | | VSS** |
| 32 | WW01154 | | EVQLVESGGGLVQPGNSLRLSCAASGFTFSKFG |
| | | | MSWVRQAPGKGLEWVSSISGSGRDTLYAESV |
| | | | KGRFTISRDNAKTTLYLQMNSLRPEDTAVYYC |
| | | | TIGGSLSVSSQGTLVTVSSsggpALFKSSFPpgsIS |
| | | | YDSPDYTDESCTFKISLRNFRSILSWELKNHSIV |
| | | | PTHYTLLYTIMSKPEDLKVVKNCANTTRSFCD |
| | | | LTDEWRSTHEAYVTVLEGFSGNTTLFSCSHNF |
| | | | WLAIDMSFEPPEFEIVGFTNHINVMVKFPSIVEE |
| | | | ELQFDLSLVIEEQSEGIVKKHKPEIKGNMSGNF |
| | | | TYIIDKLIPNTNYCVSVYLEHSDEQAVIKSPLKC |
| | | | TLLPPGQESESAESAKsggpALFKSSFPpgscdlpqth |
| | | | slgsrrtlmllaqmrrislfsclkdrhdfgfpqeefgnqfqkaetipvlhe |
| | | | miqqifnlfstkdssaawdetlldkfytelyqqlndleacviqgvgvtet |
| | | | plmkedsilavrkyfqritlylkekkyspcawevvraeimrsfslstnlq |
| | | | eslrske** |
| 33 | WW01155 | | EVQLVESGGGLVQPGNSLRLSCAASGFTFSKFG |
| | | | MSWVRQAPGKGLEWVSSISGSGRDTLYAESV |
| | | | KGRFTISRDNAKTTLYLQMNSLRPEDTAVYYC |
| | | | TIGGSLSVSSQGTLVTVSSsggpALFKSSFPpgsKN |
| | | | LKSPQKVEVDIIDDNFILRWNRSDESVGNVTFS |
| | | | FDYQKTGMDNWIKLSGCQNITSTKCNFSSLKL |
| | | | NVYEEIKLRIRAEKENTSSWYEVDSFTPFRKAQ |
| | | | IGPPEVHLEAEDKAIVIHISPGTKDSVMWALDG |
| | | | LSFTYSLVIWKNSSGVEERIENIYSRHKIYKLSP |
| | | | ETTYCLKVKAALLTSWKIGVYSPVHCIKTTVE |
| | | | NELPPPENIEVSVQNQNYVLKWDYTYANMTF |
| | | | QVQWLHAFLKRNPGNHLYKWKQIPDCENVKT |
| | | | TQCVFPQNVFQKGIYLLRVQASDGNNTSFWSE |
| | | | EIKFDTEIQsggpALFKSSFPpgscdlpqthslgsrrtlmllaq |
| | | | mrrislfsclkdrhdfgfpqeefgnqfqkaetipvlhemiqqifnlfstk |
| | | | dssaawdetlldkfytelyqqlndleacviqgvgvtetplmkedsilavr |
| | | | kyfgritlylkekkyspcawevvraeimrsfslstnlqeslrske** |
| 34 | WW01156 | | EVQLVESGGGLVQPGNSLRLSCAASGFTFSKFG |
| | | | MSWVRQAPGKGLEWVSSISGSGRDTLYAESV |
| | | | KGRFTISRDNAKTTLYLQMNSLRPEDTAVYYC |
| | | | TIGGSLSVSSQGTLVTVSSsggpALFKSSFPpgsSL |
| | | | ETITPSAFDGYPDEPCTINITIRNSRLILSWELEN |
| | | | KSGPPANYTLWYTVMSKDENLTKVKNCSDTT |
| | | | KSSCDVTDKWLEGMESYVVAIVIVHRGDLTVC |
| | | | RCSDYIVPANAPLEPPEFEIVGFTDHINVTMEFP |
| | | | PVTSKIIQEKMKTTPFVIKEQIGDSVRKKHEPKV |
| | | | NNVTGNFTFVLRDLLPKTNYCVSLYFDDDPAI |
| | | | KSPLKCIVLQPGQESGLSESAsggpALFKSSFPpgs |
| | | | cdlpqthnlrnkraltllvqmrrlsplsclkdrkdfgfpqekvdaqqikk |
| | | | aqaipvlseltqqilniftskdssaawnttlldsfcndlhqqlndlqgclm |
| | | | qqvgvqefpltqedallavrkyfhritvylrekkhspcawevvraevw |
| | | | ralsssanvlgrlreek** |
| 35 | WW01157 | | EVQLVESGGGLVQPGNSLRLSCAASGFTFSKFG |
| | | | MSWVRQAPGKGLEWVSSISGSGRDTLYAESV |
| | | | KGRFTISRDNAKTTLYLQMNSLRPEDTAVYYC |
| | | | TIGGSLSVSSQGTLVTVSSsggpALFKSSFPpgsEN |
| | | | LKPPENIDVYIIDDNYTLKWSSHGESMGSVTFS |
| | | | AEYRTKDEAKWLKVPECQHTTTTKCEFSLLDT |
| | | | NVYIKTQFRVRAEEGNSTSSWNEVDPFIPFYTA |
| | | | HMSPPEVRLEAEDKAILVHISPPGQDGNMWAL |
| | | | EKPSFSYTIRIWQKSSSDKKTINSTYYVEKIPEL |
| | | | LPETTYCLEVKAIHPSLKKHSNYSTVQCISTTV |
| | | | ANKMPVPGNLQVDAQGKSYVLKWDYIASAD |
| | | | VLFRAQWLPGYSKSSSGSRSDKWKPIPTCANV |
| | | | QTTHCVFSQDTVYTGTFFLHVQASEGNHTSFW |
| | | | SEEKFIDSQKHsggpALFKSSFPpgscdlpqthnlrnkralt |
| | | | llvqmrrlsplsclkdrkdfgfpqekvdaqqikkaqaipvlseltqqilni |
| | | | ftskdssaawnttlldsfcndlhqqlndlqgclmqqvgvqefpltqeda |
| | | | llavrkyfhritvylrekkhspcawevvraevwralsssanvlgrlreek |
| | | | ** |
| 36-194 | | | Place Hold |
| 195 | | MMP14_ | GPAGLYAQ |
| 196 | | MMP9_1 | GPAGMKGL |
| 197 | | FAPa_1 | PGGPAGIG |
| 198 | | CTSL1_1 | ALFKSSFP |
| 199 | | CTSL1_2 | ALFFSSPP |
| 200 | | ADAM17_1 | LAQRLRSS |
| 201 | | ADAM17_2 | LAQKLKSS |
| 202 | | ALU30-1 | GALFKSSFPSGGGPAGLYAQGGSGKGGSGK |
| 203 | | ALU30-2 | RGSGGGPAGLYAQGSGGGPAGLYAQGGSGK |
| 204 | | ALU30-3 | KGGGPAGLYAQGPAGLYAQGPAGLYAQGSR |
| 205 | | ALU30-4 | RGGPAGLYAQGGPAGLYAQGGGPAGLYAQK |
| 206 | | ALU30-5 | KGGALFKSSFPGGPAGIGPLAQKLKSSGGS |
| 207 | | ALU30-6 | SGGPGGPAGIGALFKSSFPLAQKLKSSGGG |
| 208 | | ALU30-7 | RGPLAQKLKSSALFKSSFPGGPAGIGGGGK |
| 209 | | ALU30-8 | GGGALFKSSFPLAQKLKSSPGGPAGIGGGR |
| 210 | | ALU30-9 | RGPGGPAGIGPLAQKLKSSALFKSSFPGGG |
| 211 | | ALU30-10 | RGGPLAQKLKSSPGGPAGIGALFKSSFPGK |
| 212 | | ALU30-11 | RSGGPAGLYAQALFKSSFPLAQKLKSSGGG |
| 213 | | ALU30-12 | GGPLAQKLKSSALFKSSFPGPAGLYAQGGR |
| 214 | | ALU30-13 | GGALFKSSFPGPAGLYAQPLAQKLKSSGGK |
| 215 | | ALU30-14 | RGGALFKSSFPLAQKLKSSGPAGLYAQGGK |
| 216 | | ALU30-15 | RGGGPAGLYAQPLAQKLKSSALFKSSFPGG |
| 217 | | ALU30-16 | SGPLAQKLKSSGPAGLYAQALFKSSFPGSK |
| 218 | | ALU30-17 | KGGPGGPAGIGPLAQRLRSSALFKSSFPGR |
| 219 | | ALU30-18 | KSGPGGPAGIGALFFSSPPLAQKLKSSGGR |
| 220 | | ALU30-19 | SGGFPRSGGSFNPRTFGSKRKRRGSRGGGG |
| 221 | | MMP14 substrate | GPLGLKAQ |
| | | motif sequence | |
| 222 | | MMP14 substrate | LPLGLKAQ |
| | | motif sequence | |
| 223 | | MMP14 substrate | SPLGLKAQ |
| | | motif sequence | |
| 224 | | MMP14 substrate | QPLGLKAQ |
| | | motif sequence | |
| 225 | | MMP14 substrate | KPLGLKAQ |
| | | motif sequence | |
| 226 | | MMP14 substrate | FPLGLKAQ |
| | | motif sequence | |
| 227 | | MMP14 substrate | HPLGLKAQ |
| | | motif sequence | |
| 228 | | MMP14 substrate | PPLGLKAQ |
| | | motif sequence | |
| 229 | | MMP14 substrate | APLGLKAQ |
| | | motif sequence | |
| 230 | | MMP14 substrate | DPLGLKAQ |
| | | motif sequence | |
| 231 | | MMP14 substrate | GPHGLKAQ |
| | | motif sequence | |
| 232 | | MMP14 substrate | GPSGLKAQ |
| | | motif sequence | |
| 233 | | MMP14 substrate | GPQGLKAQ |
| | | motif sequence | |
| 234 | | MMP14 substrate | GPPGLKAQ |
| | | motif sequence | |
| 235 | | MMP14 substrate | GPEGLKAQ |
| | | motif sequence | |
| 236 | | MMP14 substrate | GPFGLKAQ |
| | | motif sequence | |
| 237 | | MMP14 substrate | GPRGLKAQ |
| | | motif sequence | |
| 238 | | MMP14 substrate | GPGGLKAQ |
| | | motif sequence | |
| 239 | | MMP14 substrate | GPAGLKAQ |
| | | motif sequence | |
| 240 | | MMP14 substrate | LPAGLKGA |
| | | motif sequence | |
| 241 | | MMP14 substrate | GPAGLYAQ |
| | | motif sequence | |
| 242 | | MMP14 substrate | GPANLVAQ |
| | | motif sequence | |
| 243 | | MMP14 substrate | GPAALVGA |
| | | motif sequence | |
| 244 | | MMP14 substrate | GPANLRAQ |
| | | motif sequence | |
| 245 | | MMP14 substrate | GPAGLRAQ |
| | | motif sequence | |
| 246 | | MMP14 substrate | GPAGLVAQ |
| | | motif sequence | |
| 247 | | MMP14 substrate | GPAGLRGA |
| | | motif sequence | |
| 248 | | MMP14 substrate | LPAGLVGA |
| | | motif sequence | |
| 249 | | MMP14 substrate | GPAGLKGA |
| | | motif sequence | |
| 250 | | MMP14 substrate | GPLALKAQ |
| | | motif sequence | |
| 251 | | MMP14 substrate | GPLNLKAQ |
| | | motif sequence | |
| 252 | | MMP14 substrate | GPLHLKAQ |
| | | motif sequence | |
| 253 | | MMP14 substrate | GPLYLKAQ |
| | | motif sequence | |
| 254 | | MMP14 substrate | GPLPLKAQ |
| | | motif sequence | |
| 255 | | MMP14 substrate | GPLELKAQ |
| | | motif sequence | |
| 256 | | MMP14 substrate | GPLRLKAQ |
| | | motif sequence | |
| 257 | | MMP14 substrate | GPLLLKAQ |
| | | motif sequence | |
| 258 | | MMP14 substrate | GPLSLKAQ |
| | | motif sequence | |
| 259 | | MMP14 substrate | GPLGLYAQ |
| | | motif sequence | |
| 260 | | MMP14 substrate | GPLGLFAQ |
| | | motif sequence | |
| 261 | | MMP14 substrate | GPLGLLAQ |
| | | motif sequence | |
| 262 | | MMP14 substrate | GPLGLHAQ |
| | | motif sequence | |
| 263 | | MMP14 substrate | GPLGLRAQ |
| | | motif sequence | |
| 264 | | MMP14 substrate | GPLGLAAQ |
| | | motif sequence | |
| 265 | | MMP14 substrate | GPLGLEAQ |
| | | motif sequence | |
| 266 | | MMP14 substrate | GPLGLGAQ |
| | | motif sequence | |
| 267 | | MMP14 substrate | GPLGLPAQ |
| | | motif sequence | |
| 268 | | MMP14 substrate | GPLGLQAQ |
| | | motif sequence | |
| 269 | | MMP14 substrate | GPLGLSAQ |
| | | motif sequence | |
| 270 | | MMP14 substrate | GPLGLVAQ |
| | | motif sequence | |
| 271 | | MMP14 substrate | GPLGLKLQ |
| | | motif sequence | |
| 272 | | MMP14 substrate | GPLGLKFQ |
| | | motif sequence | |
| 273 | | MMP14 substrate | GPLGLKEQ |
| | | motif sequence | |
| 274 | | MMP14 substrate | GPLGLKKQ |
| | | motif sequence | |
| 275 | | MMP14 substrate | GPLGLKQQ |
| | | motif sequence | |
| 276 | | MMP14 substrate | GPLGLKSQ |
| | | motif sequence | |
| 277 | | MMP14 substrate | GPLGLKGQ |
| | | motif sequence | |
| 278 | | MMP14 substrate | GPLGLKHQ |
| | | motif sequence | |
| 279 | | MMP14 substrate | GPLGLKPQ |
| | | motif sequence | |
| 280 | | MMP14 substrate | GPLGLKAG |
| | | motif sequence | |
| 281 | | MMP14 substrate | GPLGLKAF |
| | | motif sequence | |
| 282 | | MMP14 substrate | GPLGLKAP |
| | | motif sequence | |
| 283 | | MMP14 substrate | GPLGLKAL |
| | | motif sequence | |
| 284 | | MMP14 substrate | GPLGLKAE |
| | | motif sequence | |
| 285 | | MMP14 substrate | GPLGLKAA |
| | | motif sequence | |
| 286 | | MMP14 substrate | GPLGLKAH |
| | | motif sequence | |
| 287 | | MMP14 substrate | GPLGLKAK |
| | | motif sequence | |
| 288 | | MMP14 substrate | GPLGLKAS |
| | | motif sequence | |
| 289 | | MMP14 substrate | GPLGLFGA |
| | | motif sequence | |
| 290 | | MMP14 substrate | GPLGLQGA |
| | | motif sequence | |
| 291 | | MMP14 substrate | GPLGLVGA |
| | | motif sequence | |
| 292 | | MMP14 substrate | GPLGLAGA |
| | | motif sequence | |
| 293 | | MMP14 substrate | GPLGLLGA |
| | | motif sequence | |
| 294 | | MMP14 substrate | GPLGLRGA |
| | | motif sequence | |
| 295 | | MMP14 substrate | GPLGLYGA |
| | | motif sequence | |
| 296 | | CTSL1 substrate | ALFKSSPP |
| | | motif sequence | |
| 297 | | CTSL1 substrate | SPFRSSRQ |
| | | motif sequence | |
| 298 | | CTSL1 substrate | KLFKSSPP |
| | | motif sequence | |
| 299 | | CTSL1 substrate | HLFKSSPP |
| | | motif sequence | |
| 300 | | CTSL1 substrate | SLFKSSPP |
| | | motif sequence | |
| 301 | | CTSL1 substrate | QLFKSSPP |
| | | motif sequence | |
| 302 | | CTSL1 substrate | LLFKSSPP |
| | | motif sequence | |
| 303 | | CTSL1 substrate | PLFKSSPP |
| | | motif sequence | |
| 304 | | CTSL1 substrate | FLFKSSPP |
| | | motif sequence | |
| 305 | | CTSL1 substrate | GLFKSSPP |
| | | motif sequence | |
| 306 | | CTSL1 substrate | VLFKSSPP |
| | | motif sequence | |
| 307 | | CTSL1 substrate | ELFKSSPP |
| | | motif sequence | |
| 308 | | CTSL1 substrate | AKFKSSPP |
| | | motif sequence | |
| 309 | | CTSL1 substrate | AHFKSSPP |
| | | motif sequence | |
| 310 | | CTSL1 substrate | AGFKSSPP |
| | | motif sequence | |
| 311 | | CTSL1 substrate | APFKSSPP |
| | | motif sequence | |
| 312 | | CTSL1 substrate | ANFKSSPP |
| | | motif sequence | |
| 313 | | CTSL1 substrate | AFFKSSPP |
| | | motif sequence | |
| 314 | | CTSL1 substrate | AAFKSSPP |
| | | motif sequence | |
| 315 | | CTSL1 substrate | ASFKSSPP |
| | | motif sequence | |
| 316 | | CTSL1 substrate | AEFKSSPP |
| | | motif sequence | |
| 317 | | CTSL1 substrate | ALRKSSPP |
| | | motif sequence | |
| 318 | | CTSL1 substrate | ALLKSSPP |
| | | motif sequence | |
| 319 | | CTSL1 substrate | ALAKSSPP |
| | | motif sequence | |
| 320 | | CTSL1 substrate | ALQKSSPP |
| | | motif sequence | |
| 321 | | CTSL1 substrate | ALHKSSPP |
| | | motif sequence | |
| 322 | | CTSL1 substrate | ALPKSSPP |
| | | motif sequence | |
| 323 | | CTSL1 substrate | ALTKSSPP |
| | | motif sequence | |
| 324 | | CTSL1 substrate | ALGKSSPP |
| | | motif sequence | |
| 325 | | CTSL1 substrate | ALDKSSPP |
| | | motif sequence | |
| 326 | | CTSL1 substrate | ALFFSSPP |
| | | motif sequence | |
| 327 | | CTSL1 substrate | ALFHSSPP |
| | | motif sequence | |
| 328 | | CTSL1 substrate | ALFTSSPP |
| | | motif sequence | |
| 329 | | CTSL1 substrate | ALFASSPP |
| | | motif sequence | |
| 330 | | CTSL1 substrate | ALFQSSPP |
| | | motif sequence | |
| 331 | | CTSL1 substrate | ALFLSSPP |
| | | motif sequence | |
| 332 | | CTSL1 substrate | ALFGSSPP |
| | | motif sequence | |
| 333 | | CTSL1 substrate | ALFESSPP |
| | | motif sequence | |
| 334 | | CTSL1 substrate | ALFPSSPP |
| | | motif sequence | |
| 335 | | CTSL1 substrate | ALFKHSPP |
| | | motif sequence | |
| 336 | | CTSL1 substrate | ALFKLSPP |
| | | motif sequence | |
| 337 | | CTSL1 substrate | ALFKKSPP |
| | | motif sequence | |
| 338 | | CTSL1 substrate | ALFKASPP |
| | | motif sequence | |
| 339 | | CTSL1 substrate | ALFKISPP |
| | | motif sequence | |
| 340 | | CTSL1 substrate | ALFKGSPP |
| | | motif sequence | |
| 341 | | CTSL1 substrate | ALFKNSPP |
| | | motif sequence | |
| 342 | | CTSL1 substrate | ALFKRSPP |
| | | motif sequence | |
| 343 | | CTSL1 substrate | ALFKESPP |
| | | motif sequence | |
| 344 | | CTSL1 substrate | ALFKFSPP |
| | | motif sequence | |
| 345 | | CTSL1 substrate | ALFKPSPP |
| | | motif sequence | |
| 346 | | CTSL1 substrate | ALFKSFPP |
| | | motif sequence | |
| 347 | | CTSL1 substrate | ALFKSLPP |
| | | motif sequence | |
| 348 | | CTSL1 substrate | ALFKSIPP |
| | | motif sequence | |
| 349 | | CTSL1 substrate | ALFKSKPP |
| | | motif sequence | |
| 350 | | CTSL1 substrate | ALFKSAPP |
| | | motif sequence | |
| 351 | | CTSL1 substrate | ALFKSQPP |
| | | motif sequence | |
| 352 | | CTSL1 substrate | ALFKSPPP |
| | | motif sequence | |
| 353 | | CTSL1 substrate | ALFKSEPP |
| | | motif sequence | |
| 354 | | CTSL1 substrate | ALFKSGPP |
| | | motif sequence | |
| 355 | | CTSL1 substrate | ALFKSSFP |
| | | motif sequence | |
| 356 | | CTSL1 substrate | ALFKSSLP |
| | | motif sequence | |
| 357 | | CTSL1 substrate | ALFKSSGP |
| | | motif sequence | |
| 358 | | CTSL1 substrate | ALFKSSSP |
| | | motif sequence | |
| 359 | | CTSL1 substrate | ALFKSSVP |
| | | motif sequence | |
| 360 | | CTSL1 substrate | ALFKSSHP |
| | | motif sequence | |
| 361 | | CTSL1 substrate | ALFKSSAP |
| | | motif sequence | |
| 362 | | CTSL1 substrate | ALFKSSNP |
| | | motif sequence | |
| 363 | | CTSL1 substrate | ALFKSSKP |
| | | motif sequence | |
| 364 | | CTSL1 substrate | ALFKSSEP |
| | | motif sequence | |
| 365 | | CTSL1 substrate | ALFKSSPF |
| | | motif sequence | |
| 366 | | CTSL1 substrate | ALFKSSPH |
| | | motif sequence | |
| 367 | | CTSL1 substrate | ALFKSSPG |
| | | motif sequence | |
| 368 | | CTSL1 substrate | ALFKSSPA |
| | | motif sequence | |
| 369 | | CTSL1 substrate | ALFKSSPS |
| | | motif sequence | |
| 370 | | CTSL1 substrate | ALFKSSPV |
| | | motif sequence | |
| 371 | | CTSL1 substrate | ALFKSSPQ |
| | | motif sequence | |
| 372 | | CTSL1 substrate | ALFKSSPK |
| | | motif sequence | |
| 373 | | CTSL1 substrate | ALFKSSPL |
| | | motif sequence | |
| 374 | | CTSL1 substrate | ALFKSSPD |
| | | motif sequence | |
| 375 | | MMP7 | KRALGLPG |
| 376 | | MMP7 | (DE)8RPLALWRS(DR)8 |
| | | MMP9 | PR(S/T)(L/I)(S/T) |
| 378 | | MMP9 | LEATA |
| 379 | | MMP11 | GGAANLVRGG |
| 380 | | MMP14 | SGRIGFLRTA |
| 381 | | MMP | PLGLAG |
| 382 | | MMP | PLGLAX |
| 383 | | MMP | PLGC(me)AG |
| 384 | | MMP | ESPAYYTA |
| 385 | | MMP | RLQLKL |
| 386 | | MMP | RLQLKAC |
| 387 | | MMP2, MMP9, | EP(Cit)G(Hof)YL |
| | | MMP14 | |
| 388 | | Urokinase | SGRSA |
| | | plasminogen | |
| | | activator (uPA) | |
| 389 | | Urokinase | DAFK |
| | | plasminogen | |
| | | activator (uPA) | |
| 390 | | Urokinase | GGGRR |
| | | plasminogen | |
| | | activator (uPA) | |
| 391 | | Lysosomal | GFLG |
| | | Enzyme | |
| 392 | | Lysosomal | ALAL |
| | | Enzyme | |
| | | Lysosomal | FK |
| | | Enzyme | |
| | | Cathepsin B | NLL |
| 395 | | Cathepsin D | PIC(Et)FF |
| 396 | | Cathepsin K | GGPRGLPG |
| 397 | | Prostate Specific | HSSKLQ |
| | | Antigen | |
| 398 | | Prostate Specific | HSSKLQL |
| | | Antigen | |
| 399 | | Prostate Specific | HSSKLQEDA |
| | | Antigen | |
| 400 | | Herpes Simplex | LVLASSSFGY |
| | | Virus Protease | |
| 401 | | HIV Protease | GVSQNYPIVG |
| 402 | | CMV Protease | GVVQASCRLA |
| | | Thrombin | F(Pip)RS |
| 404 | | Thrombin | DPRSFL |
| 405 | | Thrombin | PPRSFL |
| 406 | | Caspase-3 | DEVD |
| 407 | | Caspase-3 | DEVDP |
| 408 | | Caspase-3 | KGSGDVEG |
| 409 | | Interleukin 1ß | GWEHDG |
| | | converting enzyme | |
| 410 | | Enterokinase | EDDDDKA |
| 411 | | FAP | KQEQNPGST |
| 412 | | Kallikrein 2 | GKAFRR |
| 413 | | Plasmin | DAFK |
| 414 | | Plasmin | DVLK |
| 415 | | Plasmin | DAFK |
| 416 | | TOP | ALLLALL |
| 417 | | | GPLGVRG |
| 418 | | | IPVSLRSG |
| 419 | | | VPLSLYSG |

| | |
|---|---|
| 420 | SGESPAYYTA |

1. An inducible IFN prodrug comprising at least one of each of:
a) a IFN polypeptide [A];
b) a IFN blocking element [D];
c) a half-life extension element [H]; and
c) a protease-cleavable polypeptide linker [L];
wherein the IFN polypeptide and the IFN blocking element or the half-life extension element are operably linked by the protease-cleavable polypeptide linker and the inducible IFN prodrug has attenuated IFN receptor activating activity, wherein the IFN receptor activating activity of the inducible IFN prodrug is at least about 10× less than the IFN receptor activating activity of the polypeptide that contains the IFN polypeptide that is produced by cleavage of the protease cleavable linker.

2. The inducible IFN prodrug of claim 1, wherein the IFN is IFN alpha, IFN beta, IFN gamma, a mutein, or an active fragment of the foregoing.

3. The inducible IFN prodrug of claim 2, wherein the IFN is IFN alpha.

4. The inducible IFN prodrug of any one of the preceding claims, wherein the inducible IFN prodrug has the formula: 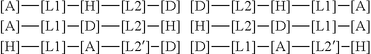
wherein [A] is a IFN polypeptide, [D] is a blocking element, [H] is a half-life extension element, [L1] is a protease-cleavable polypeptide linker, [L2] is a polypeptide linker that is optionally protease-cleavable, and [L2′] is a protease-cleavable polypeptide linker.

5. The inducible IFN prodrug of claim 2, wherein the IFN is IFN beta.

6. The inducible IFN prodrug of any one of the preceding claims, wherein the half-life extension element comprises a serum albumin binding domain, a serum albumin, transferrin, or immunoglobulin Fc, or fragment thereof.

7. The inducible IFN prodrug of any one of the preceding claims, wherein the blocking element comprises a ligand-binding domain or fragment of a cognate receptor for the IFN, an antibody or antigen-binding fragment of an antibody that binds to the IFN polypeptide.

8. The inducible IFN prodrug of claim 7, wherein the antibody or antigen-binding fragment is a single domain antibody, a Fab, or a scFv that binds the IFN polypeptide.

9. The inducible IFN prodrug of claim 7, wherein the cognate receptor for the IFN is the IFN-α/β receptor.

10. The inducible IFN prodrug of claim 7, wherein the cognate receptor for IFN is the IFNAR1 chain or the IFNAR2 chain.

11. The inducible IFN prodrug of claim 7, wherein the half-life extension element is also a blocking element.

12. The inducible IFN prodrug of any one of the preceding claims, wherein the IFN blocking element inhibits activation of the IFN receptor by the inducible IFN Prodrug.

13. The inducible IFN prodrug of any one of the preceding claims, wherein each protease-cleavable polypeptide linker independently comprises a sequence that is capable of being cleaved by a protease selected from the group consisting of a kallikrein, thrombin, chymase, carboxypeptidase A, cathepsin G, cathepsin L, an elastase, PR-3, granzyme M, a calpain, a matrix metalloproteinase (MMP), an ADAM, a FAP, a plasminogen activator, a cathepsin, a caspase, a tryptase, and a tumor cell surface protease.

14. The inducible IFN prodrug of any one of the preceding claims, wherein L2 is a protease-cleavable polypeptide linker.

15. The inducible IFN prodrug of any one of the preceding claims, wherein L1 or L2 or both L1 and L2 are cleaved by two or more different proteases.

16. The inducible IFN prodrug of claim 13, wherein the cathepsin is cathepsin B, cathepsin C, cathepsin D, cathepsin E, cathepsin K, cathepsin L, cathepsin S, or cathepsin G.

17. The inducible IFN prodrug of claim 13, wherein the matrix metalloprotease (MMP) is MMP1, MMP2, MMP3, MMP8, MMP9, MMP10, MMP11, MMP12, MMP13, MMP14, MMP19, or MMP20.

18. A nucleic acid encoding the inducible IFN prodrug of any one of the preceding claims.

19. A vector comprising the nucleic acid of claim 18.

20. A host cell comprising the vector of claim 19.

21. A pharmaceutical composition comprising a inducible IFN prodrug of any one of claims 1-17, a nucleic acid of claim 18, a vector of claim 19, or a host cell of claim 20.

22. A method of making a pharmaceutical composition, comprising culturing the host cell of claim 20 under conditions suitable for expression of the inducible IFN prodrug, and optionally isolating the inducible IFN prodrug.

23. A method for treating a cancer or a viral infection associated with cancer comprising administering to a subject in need thereof the pharmaceutical composition of claim 21.

24. A method for treating a tumor, comprising administering to a subject in need thereof an effective amount of a inducible IFN prodrug comprising at least one of each of:
a) an IFN polypeptide [A];
b) a half-life extension element [B];
c) an IFN blocking moiety [D]; and
d) a protease-cleavable polypeptide linker [L]; and
wherein the IFN polypeptide and the IFN blocking moiety or half-life extension element are operably linked by the protease-cleavable polypeptide linker and the inducible IFN prodrug has attenuated IFN-receptor activating activity, wherein the IFN-receptor activating activity of the fusion polypeptide is at least about 10 fold less than the IFN-receptor activating activity of the polypeptide that comprises the IFN polypeptide that is produced by cleavage of the protease-cleavable polypeptide linker.

25. The method of claim 24, wherein the method comprises administering an effective amount of the inducible IFN prodrug intravenously.


SEQUENCE LISTING (448 sequences)

SEQ ID NO: 1 - protein, 553 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSGGGGSEVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSS

SEQ ID NO: 2 - protein, 555 aa, synthetic construct
EVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 3 - protein, 218 aa, synthetic construct
DIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 549 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSGGGGSGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 5 - protein, 215 aa, synthetic construct
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 527 aa, synthetic construct
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 7 - protein, 632 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSGGGGSGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQ

SEQ ID NO: 8 - protein, 617 aa, synthetic construct
KNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQGGGGSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 9 - protein, 884 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKGGGGSGGGGSGGGGSGGGGSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSGGGGSGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQ

SEQ ID NO: 10 - protein, 884 aa, synthetic construct
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKGGGGSGGGGSGGGGSGGGGSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSGGGGSGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 11 - protein, 874 aa, synthetic construct
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKGGGGSGGGGSGGGGSSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 12 - protein, 635 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSGGGGSGGGGSGGGGSGGGGSENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKH

SEQ ID NO: 13 - protein, 620 aa, synthetic construct
ENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHGGGGSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 14 - protein, 891 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSSLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESAGGGGSGGGGSGGGGSGGGGSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSGGGGSGGGGSGGGGSGGGGSENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKH

SEQ ID NO: 15 - protein, 891 aa, synthetic construct
SLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESAGGGGSGGGGSGGGGSGGGGSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSGGGGSGGGGSGGGGSGGGGSENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 16 - protein, 881 aa, synthetic construct
SLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESAGGGGSGGGGSGGGGSSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSGGGGSENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 17 - protein, 231 aa, synthetic construct
EVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTHHHHHH

SEQ ID NO: 18 - protein, 568 aa, synthetic construct
DIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSGGGGSEVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSGGGGSGGGGSGGGGSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 19 - protein, 568 aa, synthetic construct
EVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSGGGGSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 20 - protein, 542 aa, synthetic construct
SLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESAGGGGSGGGGSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 21 - protein, 578 aa, synthetic construct
EVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSGGGGSGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 22 - protein, 558 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSGGGGSGGGGSEVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 23 - protein, 558 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSGGGGSGGGGSEVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSS

SEQ ID NO: 24 - protein, 528 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 528 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 26 - protein, 398 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 355 aa, synthetic construct
EVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 28 - protein, 528 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 29 - protein, 355 aa, synthetic construct
EVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 30 - protein, 578 aa, synthetic construct
EVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSGGGGSGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 31 - protein, 558 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSGGGGSGGGGSEVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 32 - protein, 527 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 33 - protein, 612 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 34 - protein, 532 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSSLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESASGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 35 - protein, 615 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 36-194 - skipped (159 entries)

SEQ ID NO: 195 - protein, 8 aa, synthetic construct
GPAGLYAQ

SEQ ID NO: 196 - protein, 8 aa, synthetic construct
GPAGMKGL

SEQ ID NO: 197 - protein, 8 aa, synthetic construct
PGGPAGIG

SEQ ID NO: 198 - protein, 8 aa, synthetic construct
ALFKSSFP

SEQ ID NO: 199 - protein, 8 aa, synthetic construct
ALFFSSPP

SEQ ID NO: 200 - protein, 8 aa, synthetic construct
LAQRLRSS

SEQ ID NO: 201 - protein, 8 aa, synthetic construct
LAQKLKSS

SEQ ID NO: 202 - protein, 30 aa, synthetic construct
GALFKSSFPSGGGPAGLYAQGGSGKGGSGK

SEQ ID NO: 203 - protein, 30 aa, synthetic construct
RGSGGGPAGLYAQGSGGGPAGLYAQGGSGK

SEQ ID NO: 204 - protein, 30 aa, synthetic construct
KGGGPAGLYAQGPAGLYAQGPAGLYAQGSR

SEQ ID NO: 205 - protein, 30 aa, synthetic construct
RGGPAGLYAQGGPAGLYAQGGGPAGLYAQK

SEQ ID NO: 206 - protein, 30 aa, synthetic construct
KGGALFKSSFPGGPAGIGPLAQKLKSSGGS

SEQ ID NO: 207 - protein, 30 aa, synthetic construct
SGGPGGPAGIGALFKSSFPLAQKLKSSGGG

SEQ ID NO: 208 - protein, 30 aa, synthetic construct
RGPLAQKLKSSALFKSSFPGGPAGIGGGGK

SEQ ID NO: 209 - protein, 30 aa, synthetic construct
GGGALFKSSFPLAQKLKSSPGGPAGIGGGR

SEQ ID NO: 210 - protein, 30 aa, synthetic construct
RGPGGPAGIGPLAQKLKSSALFKSSFPGGG

SEQ ID NO: 211 - protein, 30 aa, synthetic construct
RGGPLAQKLKSSPGGPAGIGALFKSSFPGK

SEQ ID NO: 212 - protein, 30 aa, synthetic construct
RSGGPAGLYAQALFKSSFPLAQKLKSSGGG

SEQ ID NO: 213 - protein, 30 aa, synthetic construct
GGPLAQKLKSSALFKSSFPGPAGLYAQGGR

SEQ ID NO: 214 - protein, 30 aa, synthetic construct
GGALFKSSFPGPAGLYAQPLAQKLKSSGGK

SEQ ID NO: 215 - protein, 30 aa, synthetic construct
RGGALFKSSFPLAQKLKSSGPAGLYAQGGK

SEQ ID NO: 216 - protein, 30 aa, synthetic construct
RGGGPAGLYAQPLAQKLKSSALFKSSFPGG

SEQ ID NO: 217 - protein, 30 aa, synthetic construct
SGPLAQKLKSSGPAGLYAQALFKSSFPGSK

SEQ ID NO: 218 - protein, 30 aa, synthetic construct
KGGPGGPAGIGPLAQRLRSSALFKSSFPGR

SEQ ID NO: 219 - protein, 30 aa, synthetic construct
KSGPGGPAGIGALFFSSPPLAQKLKSSGGR

SEQ ID NO: 220 - protein, 30 aa, synthetic construct
SGGFPRSGGSFNPRTFGSKRKRRGSRGGGG

SEQ ID NO: 221 - protein, 8 aa, synthetic construct
GPLGLKAQ

SEQ ID NO: 222 - protein, 8 aa, synthetic construct
LPLGLKAQ

SEQ ID NO: 223 - protein, 8 aa, synthetic construct
SPLGLKAQ

SEQ ID NO: 224 - protein, 8 aa, synthetic construct
QPLGLKAQ

SEQ ID NO: 225 - protein, 8 aa, synthetic construct
KPLGLKAQ

SEQ ID NO: 226 - protein, 8 aa, synthetic construct
FPLGLKAQ

SEQ ID NO: 227 - protein, 8 aa, synthetic construct
HPLGLKAQ

SEQ ID NO: 228 - protein, 8 aa, synthetic construct
PPLGLKAQ

SEQ ID NO: 229 - protein, 8 aa, synthetic construct
APLGLKAQ

SEQ ID NO: 230 - protein, 8 aa, synthetic construct
DPLGLKAQ

SEQ ID NO: 231 - protein, 8 aa, synthetic construct
GPHGLKAQ

SEQ ID NO: 232 - protein, 8 aa, synthetic construct
GPSGLKAQ

SEQ ID NO: 233 - protein, 8 aa, synthetic construct
GPQGLKAQ

SEQ ID NO: 234 - protein, 8 aa, synthetic construct
GPPGLKAQ

SEQ ID NO: 235 - protein, 8 aa, synthetic construct
GPEGLKAQ

SEQ ID NO: 236 - protein, 8 aa, synthetic construct
GPFGLKAQ

SEQ ID NO: 237 - protein, 8 aa, synthetic construct
GPRGLKAQ

SEQ ID NO: 238 - protein, 8 aa, synthetic construct
GPGGLKAQ

SEQ ID NO: 239 - protein, 8 aa, synthetic construct
GPAGLKAQ

SEQ ID NO: 240 - protein, 8 aa, synthetic construct
LPAGLKGA

SEQ ID NO: 241 - protein, 8 aa, synthetic construct
GPAGLYAQ

SEQ ID NO: 242 - protein, 8 aa, synthetic construct
GPANLVAQ

SEQ ID NO: 243 - protein, 8 aa, synthetic construct
GPAALVGA

SEQ ID NO: 244 - protein, 8 aa, synthetic construct
GPANLRAQ

SEQ ID NO: 245 - protein, 8 aa, synthetic construct
GPAGLRAQ

SEQ ID NO: 246 - protein, 8 aa, synthetic construct
GPAGLVAQ

SEQ ID NO: 247 - protein, 8 aa, synthetic construct
GPAGLRGA

SEQ ID NO: 248 - protein, 8 aa, synthetic construct
LPAGLVGA

SEQ ID NO: 249 - protein, 8 aa, synthetic construct
GPAGLKGA

SEQ ID NO: 250 - protein, 8 aa, synthetic construct
GPLALKAQ

SEQ ID NO: 251 - protein, 8 aa, synthetic construct
GPLNLKAQ

SEQ ID NO: 252 - protein, 8 aa, synthetic construct
GPLHLKAQ

SEQ ID NO: 253 - protein, 8 aa, synthetic construct
GPLYLKAQ

SEQ ID NO: 254 - protein, 8 aa, synthetic construct
GPLPLKAQ

SEQ ID NO: 255 - protein, 8 aa, synthetic construct
GPLELKAQ

SEQ ID NO: 256 - protein, 8 aa, synthetic construct
GPLRLKAQ

SEQ ID NO: 257 - protein, 8 aa, synthetic construct
GPLLLKAQ

SEQ ID NO: 258 - protein, 8 aa, synthetic construct
GPLSLKAQ

SEQ ID NO: 259 - protein, 8 aa, synthetic construct
GPLGLYAQ

SEQ ID NO: 260 - protein, 8 aa, synthetic construct
GPLGLFAQ

SEQ ID NO: 261 - protein, 8 aa, synthetic construct
GPLGLLAQ

SEQ ID NO: 262 - protein, 8 aa, synthetic construct
GPLGLHAQ

SEQ ID NO: 263 - protein, 8 aa, synthetic construct
GPLGLRAQ

SEQ ID NO: 264 - protein, 8 aa, synthetic construct
GPLGLAAQ

SEQ ID NO: 265 - protein, 8 aa, synthetic construct
GPLGLEAQ

SEQ ID NO: 266 - protein, 8 aa, synthetic construct
GPLGLGAQ

SEQ ID NO: 267 - protein, 8 aa, synthetic construct
GPLGLPAQ

SEQ ID NO: 268 - protein, 8 aa, synthetic construct
GPLGLQAQ

SEQ ID NO: 269 - protein, 8 aa, synthetic construct
GPLGLSAQ

SEQ ID NO: 270 - protein, 8 aa, synthetic construct
GPLGLVAQ

SEQ ID NO: 271 - protein, 8 aa, synthetic construct
GPLGLKLQ

SEQ ID NO: 272 - protein, 8 aa, synthetic construct
GPLGLKFQ

SEQ ID NO: 273 - protein, 8 aa, synthetic construct
GPLGLKEQ

SEQ ID NO: 274 - protein, 8 aa, synthetic construct
GPLGLKKQ

SEQ ID NO: 275 - protein, 8 aa, synthetic construct
GPLGLKQQ

SEQ ID NO: 276 - protein, 8 aa, synthetic construct
GPLGLKSQ

SEQ ID NO: 277 - protein, 8 aa, synthetic construct
GPLGLKGQ

SEQ ID NO: 278 - protein, 8 aa, synthetic construct
GPLGLKHQ

SEQ ID NO: 279 - protein, 8 aa, synthetic construct
GPLGLKPQ

SEQ ID NO: 280 - protein, 8 aa, synthetic construct
GPLGLKAG

SEQ ID NO: 281 - protein, 8 aa, synthetic construct
GPLGLKAF

SEQ ID NO: 282 - protein, 8 aa, synthetic construct
GPLGLKAP

SEQ ID NO: 283 - protein, 8 aa, synthetic construct
GPLGLKAL

SEQ ID NO: 284 - protein, 8 aa, synthetic construct
GPLGLKAE

SEQ ID NO: 285 - protein, 8 aa, synthetic construct
GPLGLKAA

SEQ ID NO: 286 - protein, 8 aa, synthetic construct
GPLGLKAH

SEQ ID NO: 287 - protein, 8 aa, synthetic construct
GPLGLKAK

SEQ ID NO: 288 - protein, 8 aa, synthetic construct
GPLGLKAS

SEQ ID NO: 289 - protein, 8 aa, synthetic construct
GPLGLFGA

SEQ ID NO: 290 - protein, 8 aa, synthetic construct
GPLGLQGA

SEQ ID NO: 291 - protein, 8 aa, synthetic construct
GPLGLVGA

SEQ ID NO: 292 - protein, 8 aa, synthetic construct
GPLGLAGA

SEQ ID NO: 293 - protein, 8 aa, synthetic construct
GPLGLLGA

SEQ ID NO: 294 - protein, 8 aa, synthetic construct
GPLGLRGA

SEQ ID NO: 295 - protein, 8 aa, synthetic construct
GPLGLYGA

SEQ ID NO: 296 - protein, 8 aa, synthetic construct
ALFKSSPP

SEQ ID NO: 297 - protein, 8 aa, synthetic construct
SPFRSSRQ

SEQ ID NO: 298 - protein, 8 aa, synthetic construct
KLFKSSPP

SEQ ID NO: 299 - protein, 8 aa, synthetic construct
HLFKSSPP

SEQ ID NO: 300 - protein, 8 aa, synthetic construct
SLFKSSPP

SEQ ID NO: 301 - protein, 8 aa, synthetic construct
QLFKSSPP

SEQ ID NO: 302 - protein, 8 aa, synthetic construct
LLFKSSPP

SEQ ID NO: 303 - protein, 8 aa, synthetic construct
PLFKSSPP

SEQ ID NO: 304 - protein, 8 aa, synthetic construct
FLFKSSPP

SEQ ID NO: 305 - protein, 8 aa, synthetic construct
GLFKSSPP

SEQ ID NO: 306 - protein, 8 aa, synthetic construct
VLFKSSPP

SEQ ID NO: 307 - protein, 8 aa, synthetic construct
ELFKSSPP

SEQ ID NO: 308 - protein, 8 aa, synthetic construct
AKFKSSPP

SEQ ID NO: 309 - protein, 8 aa, synthetic construct
AHFKSSPP

SEQ ID NO: 310 - protein, 8 aa, synthetic construct
AGFKSSPP

SEQ ID NO: 311 - protein, 8 aa, synthetic construct
APFKSSPP

SEQ ID NO: 312 - protein, 8 aa, synthetic construct
ANFKSSPP

SEQ ID NO: 313 - protein, 8 aa, synthetic construct
AFFKSSPP

SEQ ID NO: 314 - protein, 8 aa, synthetic construct
AAFKSSPP

SEQ ID NO: 315 - protein, 8 aa, synthetic construct
ASFKSSPP

SEQ ID NO: 316 - protein, 8 aa, synthetic construct
AEFKSSPP

SEQ ID NO: 317 - protein, 8 aa, synthetic construct
ALRKSSPP

SEQ ID NO: 318 - protein, 8 aa, synthetic construct
ALLKSSPP

SEQ ID NO: 319 - protein, 8 aa, synthetic construct
ALAKSSPP

SEQ ID NO: 320 - protein, 8 aa, synthetic construct
ALQKSSPP

SEQ ID NO: 321 - protein, 8 aa, synthetic construct
ALHKSSPP

SEQ ID NO: 322 - protein, 8 aa, synthetic construct
ALPKSSPP

SEQ ID NO: 323 - protein, 8 aa, synthetic construct
ALTKSSPP

SEQ ID NO: 324 - protein, 8 aa, synthetic construct
ALGKSSPP

SEQ ID NO: 325 - protein, 8 aa, synthetic construct
ALDKSSPP

SEQ ID NO: 326 - protein, 8 aa, synthetic construct
ALFFSSPP

SEQ ID NO: 327 - protein, 8 aa, synthetic construct
ALFHSSPP

SEQ ID NO: 328 - protein, 8 aa, synthetic construct
ALFTSSPP

SEQ ID NO: 329 - protein, 8 aa, synthetic construct
ALFASSPP

SEQ ID NO: 330 - protein, 8 aa, synthetic construct
ALFQSSPP

SEQ ID NO: 331 - protein, 8 aa, synthetic construct
ALFLSSPP

SEQ ID NO: 332 - protein, 8 aa, synthetic construct
ALFGSSPP

SEQ ID NO: 333 - protein, 8 aa, synthetic construct
ALFESSPP

SEQ ID NO: 334 - protein, 8 aa, synthetic construct
ALFPSSPP

SEQ ID NO: 335 - protein, 8 aa, synthetic construct
ALFKHSPP

SEQ ID NO: 336 - protein, 8 aa, synthetic construct
ALFKLSPP

SEQ ID NO: 337 - protein, 8 aa, synthetic construct
ALFKKSPP

SEQ ID NO: 338 - protein, 8 aa, synthetic construct
ALFKASPP

SEQ ID NO: 339 - protein, 8 aa, synthetic construct
ALFKISPP

SEQ ID NO: 340 - protein, 8 aa, synthetic construct
ALFKGSPP

SEQ ID NO: 341 - protein, 8 aa, synthetic construct
ALFKNSPP

SEQ ID NO: 342 - protein, 8 aa, synthetic construct
ALFKRSPP

SEQ ID NO: 343 - protein, 8 aa, synthetic construct
ALFKESPP

SEQ ID NO: 344 - protein, 8 aa, synthetic construct
ALFKFSPP

SEQ ID NO: 345 - protein, 8 aa, synthetic construct
ALFKPSPP

SEQ ID NO: 346 - protein, 8 aa, synthetic construct
ALFKSFPP

SEQ ID NO: 347 - protein, 8 aa, synthetic construct
ALFKSLPP

SEQ ID NO: 348 - protein, 8 aa, synthetic construct
ALFKSIPP

SEQ ID NO: 349 - protein, 8 aa, synthetic construct
ALFKSKPP

SEQ ID NO: 350 - protein, 8 aa, synthetic construct
ALFKSAPP

SEQ ID NO: 351 - protein, 8 aa, synthetic construct
ALFKSQPP

SEQ ID NO: 352 - protein, 8 aa, synthetic construct
ALFKSPPP

SEQ ID NO: 353 - protein, 8 aa, synthetic construct
ALFKSEPP

SEQ ID NO: 354 - protein, 8 aa, synthetic construct
ALFKSGPP

SEQ ID NO: 355 - protein, 8 aa, synthetic construct
ALFKSSFP

SEQ ID NO: 356 - protein, 8 aa, synthetic construct
ALFKSSLP

SEQ ID NO: 357 - protein, 8 aa, synthetic construct
ALFKSSGP

SEQ ID NO: 358 - protein, 8 aa, synthetic construct
ALFKSSSP

SEQ ID NO: 359 - protein, 8 aa, synthetic construct
ALFKSSVP

SEQ ID NO: 360 - protein, 8 aa, synthetic construct
ALFKSSHP

SEQ ID NO: 361 - protein, 8 aa, synthetic construct
ALFKSSAP

SEQ ID NO: 362 - protein, 8 aa, synthetic construct
ALFKSSNP

SEQ ID NO: 363 - protein, 8 aa, synthetic construct
ALFKSSKP

SEQ ID NO: 364 - protein, 8 aa, synthetic construct
ALFKSSEP

SEQ ID NO: 365 - protein, 8 aa, synthetic construct
ALFKSSPF

SEQ ID NO: 366 - protein, 8 aa, synthetic construct
ALFKSSPH

SEQ ID NO: 367 - protein, 8 aa, synthetic construct
ALFKSSPG

SEQ ID NO: 368 - protein, 8 aa, synthetic construct
ALFKSSPA

SEQ ID NO: 369 - protein, 8 aa, synthetic construct
ALFKSSPS

SEQ ID NO: 370 - protein, 8 aa, synthetic construct
ALFKSSPV

SEQ ID NO: 371 - protein, 8 aa, synthetic construct
ALFKSSPQ

SEQ ID NO: 372 - protein, 8 aa, synthetic construct
ALFKSSPK

SEQ ID NO: 373 - protein, 8 aa, synthetic construct
ALFKSSPL

SEQ ID NO: 374 - protein, 8 aa, synthetic construct
ALFKSSPD

SEQ ID NO: 375 - protein, 8 aa, synthetic construct
KRALGLPG

SEQ ID NO: 376 - protein, 40 aa, synthetic construct
DEDEDEDEDEDEDEDERPLALWRSDRDRDRDRDRDRDRDR

SEQ ID NO: 377 - skipped

SEQ ID NO: 378 - protein, 5 aa, synthetic construct
LEATA

SEQ ID NO: 379 - protein, 10 aa, synthetic construct
GGAANLVRGG

SEQ ID NO: 380 - protein, 10 aa, synthetic construct
SGRIGFLRTA

SEQ ID NO: 381 - protein, 6 aa, synthetic construct
PLGLAG

SEQ ID NO: 382 - protein, 6 aa, synthetic construct
PLGLAX

SEQ ID NO: 383 - protein, 6 aa, synthetic construct
  MOD_RES 4: note = s-methyl cysteine
PLGCAG

SEQ ID NO: 384 - protein, 8 aa, synthetic construct
ESPAYYTA

SEQ ID NO: 385 - protein, 6 aa, synthetic construct
RLQLKL

SEQ ID NO: 386 - protein, 7 aa, synthetic construct
RLQLKAC

SEQ ID NO: 387 - protein, 7 aa, synthetic construct
  MOD_RES 3: note = citrulline
  MOD_RES 5: note = homophenylalanine
EPXGXYL

SEQ ID NO: 388 - protein, 5 aa, synthetic construct
SGRSA

SEQ ID NO: 389 - protein, 4 aa, synthetic construct
DAFK

SEQ ID NO: 390 - protein, 5 aa, synthetic construct
GGGRR

SEQ ID NO: 391 - protein, 4 aa, synthetic construct
GFLG

SEQ ID NO: 392 - protein, 4 aa, synthetic construct
ALAL

SEQ ID NO: 393-394 - skipped (2 entries)

SEQ ID NO: 395 - protein, 5 aa, synthetic construct
  MOD_RES 3: note = s-ethyl cysteine
PICFF

SEQ ID NO: 396 - protein, 8 aa, synthetic construct
GGPRGLPG

SEQ ID NO: 397 - protein, 6 aa, synthetic construct
HSSKLQ

SEQ ID NO: 398 - protein, 7 aa, synthetic construct
HSSKLQL

SEQ ID NO: 399 - protein, 9 aa, synthetic construct
HSSKLQEDA

SEQ ID NO: 400 - protein, 10 aa, synthetic construct
LVLASSSFGY

SEQ ID NO: 401 - protein, 10 aa, synthetic construct
GVSQNYPIVG

SEQ ID NO: 402 - protein, 10 aa, synthetic construct
GVVQASCRLA

SEQ ID NO: 403 - skipped

SEQ ID NO: 404 - protein, 6 aa, synthetic construct
DPRSFL

SEQ ID NO: 405 - protein, 6 aa, synthetic construct
PPRSFL

SEQ ID NO: 406 - protein, 4 aa, synthetic construct
DEVD

SEQ ID NO: 407 - protein, 5 aa, synthetic construct
DEVDP

SEQ ID NO: 408 - protein, 8 aa, synthetic construct
KGSGDVEG

SEQ ID NO: 409 - protein, 6 aa, synthetic construct
GWEHDG

SEQ ID NO: 410 - protein, 7 aa, synthetic construct
EDDDDKA

SEQ ID NO: 411 - protein, 9 aa, synthetic construct
KQEQNPGST

SEQ ID NO: 412 - protein, 6 aa, synthetic construct
GKAFRR

SEQ ID NO: 413 - protein, 4 aa, synthetic construct
DAFK

SEQ ID NO: 414 - protein, 4 aa, synthetic construct
DVLK

SEQ ID NO: 415 - protein, 4 aa, synthetic construct
DAFK

SEQ ID NO: 416 - protein, 7 aa, synthetic construct
ALLLALL

SEQ ID NO: 417 - protein, 7 aa, synthetic construct
GPLGVRG

SEQ ID NO: 418 - protein, 8 aa, synthetic construct
IPVSLRSG

SEQ ID NO: 419 - protein, 8 aa, synthetic construct
VPLSLYSG

SEQ ID NO: 420 - protein, 10 aa, synthetic construct
SGESPAYYTA

SEQ ID NO: 421-446 - skipped (26 entries)

SEQ ID NO: 447 - protein, 7 aa, synthetic construct
PAGLYAQ

SEQ ID NO: 448 - protein, 7 aa, synthetic construct
LFKSSFP